(19) United States
(12) Patent Application Publication
(10) Pub. No.: US 2014/0309296 A1
(43) Pub. Date: Oct. 16, 2014

(54) BUDIODARONE FORMULATIONS

(71) Applicant: Armetheon, Inc., Menlo Park, CA (US)

(72) Inventor: Pascal J. DRUZGALA, Santa Rosa, CA (US)

(73) Assignee: ARMETHEON, INC., Menlo Park, CA (US)

(21) Appl. No.: 14/206,217

(22) Filed: Mar. 12, 2014

Related U.S. Application Data
(60) Provisional application No. 61/780,724, filed on Mar. 13, 2013.

(51) Int. Cl. A61K 31/343

(52) U.S. Cl. CPC A61K 31/343
USPC 514/469

(57) ABSTRACT

The present invention relates to budiodarone formulations which comprise a solution comprising benzyl alcohol and budiodarone, wherein the budiodarone has an aqueous solubility of about 200 mg/ml to about 1250 mg/ml when contacted with an aqueous solution. The budiodarone formulations can be highly concentrated and stored with stability, yet the formulations are also dilutable and can be delivered to a subject with ease and immediacy.

### CROSS-REFERENCE TO RELATED APPLICATIONS

This application claims the priority benefit of U.S. Provisional Patent Application Ser. No. 61/780,724, filed Mar. 13, 2013, which is incorporated herein by reference in its entirety.

### TECHNICAL FIELD

The present invention relates to budiodarone formulations which comprise a solution comprising benzyl alcohol and budiodarone, wherein the budiodarone has an aqueous solubility of about 200 mg/ml to about 1250 mg/ml when contacted with an aqueous solution. The budiodarone formulations can be highly concentrated and stored with stability, yet the formulations are also dilutable and can be delivered to a subject with ease and immediacy.

### BACKGROUND

Atrial fibrillation (AF) is a supraventricular tachyarrhythmia that affects more than 2 million adult Americans, with an incidence of about 160,000 each year. AF is characterized by rapid, irregular, and chaotic atrial activity with consequent deterioration of atrial mechanical function. The development of AF has two main consequences: hemodynamic and thromboembolic. Hemodynamic consequences include hypotension and heart failure resulting from decreased cardiac output and are more profound in patients with structural heart disease; thromboembolic consequences include stroke and systemic thromboembolism. The incidence of AF is around 5% in those over 65 years of age, rising to about 10% in people over 80. In the postoperative setting, AF is common, occurring in up to 50% of post-cardiac surgery subjects, leading to longer hospital stay and increased healthcare cost. About half of the patients presenting with acute AF spontaneously revert to sinus rhythm within 48 hours, however acute AF is associated with early ischemic stroke clustering, particularly at the time of onset, leading to a high mortality. The risk of early ischemic stroke clustering is higher in hyperthyroid patients. In stroke survivors, the rate of recurrence is high.

The three main therapeutic strategies for the management of AF are ventricular rate control, termination of the arrhythmia (cardioversion), and prevention of recurrences and thromboembolic events. Options for cardioverting patients to normal sinus rhythm include synchronized direct current cardioversion (DCC) and pharmacological cardioversion. DCC requires conscious sedation to manage the discomfort of the procedure and is associated with ventricular fibrillation in 0.2% of cases, sinus bradycardia or sinus arrest in 1% of cases, and thromboembolism in 1% of cases not adequately anticoagulated. Pharmacological cardioversion is less effective than DCC, but is more cost effective and several drugs are currently approved for this indication. These drugs carry risks similar to those of electric cardioversion with additional concerns including risks of pro-arrhythmic events. The therapeutic approach depends on whether the new-onset patients present with hemodynamic instability or not.

In patients with life-threatening hemodynamic instability, i.e., patients with ventricular rates higher than 150 bpm, ongoing chest pain, or with systolic blood pressure <90 mm Hg, heart failure, or reduced consciousness, the need to restore stability by restoring sinus rhythm is of prime importance. Guideline consensus advocates DCC.

Patients with stable acute AF have relatively less urgent needs and have alternative options. DCC is still an option, but it is an elective procedure. In most cases of recent-onset AF, pharmacological cardioversion provides an alternative to DCC, which can then be employed as a second-line therapy for non-responders. Treatment should be directed to symptom relief and the prevention of complications. Safety and efficacy considerations then become important in optimizing the choice of an antiarrhythmic drug for the treatment of AF. Current antiarrhythmic drug therapies for the conversion of atrial fibrillation and the maintenance of sinus rhythm are limited by efficacy, tolerance and safety of the currently available agents. Therefore, a goal is to develop effective antiarrhythmic drugs with as little as possible side effects. In an ideal situation, the same drug should be used intravenously to terminate acute AF and then orally thereafter to prevent recurrence of arrhythmia. In practice, there is only one drug presently available to clinicians that can serve both purposes. This drug is amiodarone hydrochloride, which has been available for several decades. Amiodarone hydrochloride however is not approved for this indication and no formal studies have ever been done in man to quantify the risks associated with this procedure. Therefore there is no available option which is FDA or EMA approved to both terminate persistent AF and prevent recurrence of symptoms using the same drug.

### SUMMARY OF THE INVENTION

The present disclosure also provides a solution comprising benzyl alcohol and budiodarone for use as a medicament. In another aspect, the present disclosure provides a solution comprising benzyl alcohol and budiodarone for use in therapy.

The present disclosure provides, in some embodiments, a solution comprising benzyl alcohol and budiodarone, wherein the budiodarone has an aqueous solubility of about 200 mg/ml to about 1250 mg/ml when contacted with an aqueous solution.

In some embodiments, the aqueous solubility of budiodarone in the solution is 200 mg/ml to 750 mg/ml. In some embodiments, the aqueous solubility of budiodarone in the solution is 750 mg/ml to 1250 mg/ml. In some embodiments, the benzyl alcohol and budiodarone are in a molar ratio of benzyl alcohol to budiodarone of between about 0.5 to 1 and 4.5 to 1. In some embodiments, the solution has characteristics of a solution prepared by: a) mixing benzyl alcohol, budiodarone, and a co-solvent in a sufficient amount to make a clear solution, wherein the benzyl alcohol and budiodarone are present in a molar ratio that is greater than between about 0.5 to 1 and 4.5 to 1; and b) removing the co-solvent until the solution comprises benzyl alcohol and budiodarone in a molar ratio of benzyl alcohol to budiodarone of between about 0.5 to 1 and 4.5 to 1. In some embodiments, the solution has characteristics of a solution prepared by: a) mixing benzyl alcohol, budiodarone, and water, wherein the benzyl alcohol and budiodarone are present in a molar ratio that is greater than between about 0.5 to 1 and 4.5 to 1; and b) removing the water by lyophilization until the formulation comprising benzyl alcohol and budiodarone in a molar ratio of benzyl alcohol to budiodarone of between about 0.5 to 1 and 4.5 to 1 is formed. In some embodiments, the benzyl alcohol and budiodarone are present when mixed in a molar ratio that is between about 0.5 to 1 and 11 to 1 in step (a). In some embodiments, the benzyl alcohol and budiodarone are present in a molar ratio of benzyl alcohol to budiodarone of between about 2 to 1 and 4 to 1. In some embodiments, the benzyl alcohol and budiodarone are present in a molar ratio of benzyl alcohol to budiodarone of about 3 to 1. In some embodiments, the benzyl alcohol and budiodarone are present in a molar ratio of benzyl alcohol to budiodarone of between about 5 to 1 and 11 to 1. In some embodiments, the solution is substantially free of a solubilizing excipient. In some embodiments, the solubilizing excipient is selected from water-soluble organic solvents (polyethylene glycol 300, polyethylene glycol 400, ethanol, propylene glycol, glycerin, N-methyl-2-pyrrolidone, dimethylacetamide, dimethylsulfoxide, etc.), non-ionic surfactants (CREMOPHOR® EL, CREMOPHOR® RH 60, polysorbate 80, SOLUTOL® HS 15, etc.), water-insoluble lipids, phospholipids (usually for injectable emulsions), and various cyclodextrins. In some embodiments, the solubilizing excipient is TWEEN. In some embodiments, the solution comprises an emulsifier selected from labrasol, gelucire, phospholipid, cholesterol, or plant sterols. In some embodiments, the solution comprising benzyl alcohol and budiodarone can be stored for at least 1-5, 5-9, 10-14, 15-19, 20-24, 25-29, 30-34, 35-39, 40-60, or 60-90 days at room temperature or up to around 40° C. In some embodiments, the solution comprising benzyl alcohol and budiodarone can be stored for at least 1, 5, 10, 15, 20, 25, 30, 31, 32, 33, 34, 35, 36, 37, 38, 39, 40, 50, 60, 70, 80, or 90 days at room temperature or up to around 40° C. In some embodiments, the solution comprising benzyl alcohol and budiodarone can be stored for at least 4, 5, 6, 7, 8, 9, 10, 11, 12, 13, 14, 15, 16, 17, 18, 19, 20, 21, 22, 23, or 24 months at room temperature or up to around 40° C. In some embodiments, the solution comprising benzyl alcohol and budiodarone can be stored for at least 3, 4, 5, 6, 7, 8, 9, or years at room temperature or up to around 40° C. In some embodiments, the solution comprising benzyl alcohol and budiodarone can be stored for at least 1 or 2 years at room temperature or up to around 40° C. In some embodiments, the solution comprising benzyl alcohol and budiodarone can be stored for at least 2 years at room temperature or up to around 40° C. In some embodiments, the solution is stored at room temperature or cold temperature.

The present disclosure provides, in some embodiments, an aqueous solution comprising benzyl alcohol and budiodarone, wherein the budiodarone is present in the solution in a concentration of about 200 mg/ml to about 1250 mg/ml.

In some embodiments, the concentration of budiodarone in the solution is between about 500 and 1250 mg/ml. In some embodiments, the concentration of budiodarone in the solution is between about 800 and 1250 mg/ml. In some embodiments, the concentration of budiodarone in the solution is between about 0.1 and 500 mg/ml. In some embodiments, benzyl alcohol is present in a concentration of 0.005% to 12.5% v/v. In some embodiments, benzyl alcohol is present in a concentration of 1.5% to 48.5% v/v. In some embodiments, benzyl alcohol is present in a concentration of 4% to 48.5% v/v. In some embodiments, the solution comprises dextrose or sucrose. In some embodiments, dextrose solution is 5% w/v. In some embodiments, the solution comprises an osmolality modifier selected from dimethylacetamide, propylene glycol, and polyethylene glycol.

The present disclosure provides, in some embodiments, a capsule comprising any of the solutions described above. In some embodiments, the capsule is a gelcap. In some embodiments, the capsule further comprises pharmaceutically acceptable excipients. In some embodiments, the amount of budiodarone in the capsule is from about 50 mg to about 800 mg.

The present disclosure provides, in some embodiments, an oral syrup comprising any of the solutions described above and pharmaceutically acceptable excipients.

The present disclosure provides, in some embodiments, a kit comprising any of the solutions described above and instructions for use. In some embodiments, the formulation in the kit is contained in an ampoule or syringe.

The present disclosure provides, in some embodiments, a process for preparing a solution comprising benzyl alcohol and budiodarone in a molar ratio of benzyl alcohol to budiodarone of between about 0.5 to 1 and 4.5 to 1, wherein the budiodarone has an aqueous solubility of about 200 mg/ml to about 1250 mg/ml when contacted with an aqueous environment, and wherein the process comprises: a) mixing benzyl alcohol, budiodarone, and a co-solvent in a sufficient amount to make a clear solution, wherein the benzyl alcohol and budiodarone are present in a molar ratio that is greater than between about 0.5 to 1 and 4.5 to 1; and b) removing the co-solvent until the solution comprises benzyl alcohol and budiodarone in a molar ratio of benzyl alcohol to budiodarone of between about 0.5 to 1 and 4.5 to 1.

In some embodiments, the benzyl alcohol and budiodarone in step (a) are present in a molar ratio of benzyl alcohol to budiodarone is between about 5 to 1 and 11 to 1 when the benzyl alcohol, budiodarone, and co-solvent are mixed. In some embodiments, the co-solvent is selected from methylene chloride, methanol, acetone, ethyl acetate, acetonitrile, and supercritical carbon dioxide. In some embodiments, the co-solvent is removed under reduced pressure. In some embodiments, the co-solvent is removed under reduced pressure and the resulting solution is left under reduced pressure until a molar ratio of between about 0.5 to 1 and 4.5 to 1 is obtained. In some embodiments, the method further comprises adding water to the solution comprising benzyl alcohol and budiodarone in a molar ratio of benzyl alcohol to budiodarone of between about 0.5 to 1 and 4.5 to 1 after step (b).

The present disclosure provides, in some embodiments, a process for preparing a solution comprising benzyl alcohol and budiodarone in a molar ratio of benzyl alcohol to budiodarone of between about 0.5 to 1 and 4.5 to 1, wherein the budiodarone has an aqueous solubility of about 200 mg/ml to about 1250 mg/ml when contacted with an aqueous environment, and wherein the process comprises: a) mixing benzyl alcohol, budiodarone, and water, wherein the benzyl alcohol and budiodarone are present in a molar ratio that is greater than between about 0.5 to 1 and 4.5 to 1; and b) removing the water by lyophilization until the formulation comprising benzyl alcohol and budiodarone in a molar ratio of benzyl alcohol to budiodarone of between about 0.5 to 1 and 4.5 to 1 is formed.

In some embodiments, the benzyl alcohol and budiodarone in step (a) are present in a molar ratio of benzyl alcohol to budiodarone that is between about 5 to 1 and 11 to 1 when the benzyl alcohol, budiodarone, and water are mixed.

The present disclosure provides, in some embodiments, a method of treating cardiac arrhythmia or reducing the incidence of cardiac arrhythmias comprising administering to a patient in need thereof any of the solutions described above.

The present disclosure provides, in some embodiments, a method of restoring sinus and ventricular rhythms in a patient having persistent atrial fibrillation, paroxysmal atrial fibrillation, atrial flutter, ventricular arrhythmias, or ventricular fibrillation comprising administering to a patient in need thereof any of the solutions described above.

The present disclosure provides, in some embodiments, a method of restoring normal cardiac rhythms in a patient comprising administering to the patient in need thereof any of the solutions described above.

The present disclosure provides, in some embodiments, a method of reducing atrial fibrillation burden in a patient comprising administering to the patient in need thereof any of the solutions described above.

The present disclosure provides, in some embodiments, a method of reducing atrial fibrillation burden in a patient comprising administering to the patient in need thereof any of the solutions described above.

The present disclosure provides, in some embodiments, a method for preventing atrial remodeling in a patient comprising administering to the patient in need thereof any of the solutions described above.

The present disclosure provides, in some embodiments, a method for reversing atrial remodeling in a patient comprising administering to the patient in need thereof any of the solutions described above.

The present disclosure provides, in some embodiments, a method for reducing stroke rate in a patient comprising administering to the patient in need thereof any of the solutions described above.

The present disclosure provides, in some embodiments, a method for increasing time in normal sinus rhythm in a patient comprising administering to the patient in need thereof any of the solutions described above.

The present disclosure provides, in some embodiments, a method for treating a refractory atrial fibrillation patient comprising administering to patient refractory to one or more anti-arrhythmic drugs any of the solutions described above in an effective amount to reduce atrial fibrillation burden (AFB).

In some embodiments in the above methods, the formulation is administered intravenously. In some embodiments in the above methods, the formulation is administered orally. In some embodiments in the above methods, the patient is a newborn. In some embodiments in the above methods, the patient is of middle age or old age.

The present disclosure provides, in some embodiments, a solution comprising benzyl alcohol and budiodarone for treating cardiac arrhythmia or reducing the incidence of cardiac arrhythmias. The present disclosure provides, in some embodiments, a solution comprising benzyl alcohol and budiodarone for restoring sinus and ventricular rhythms in a patient having persistent atrial fibrillation, paroxysmal atrial fibrillation, atrial flutter, ventricular arrhythmias, or ventricular fibrillation. The present disclosure provides, in some embodiments, a solution comprising benzyl alcohol and budiodarone for restoring normal cardiac rhythms in a patient. The present disclosure provides, in some embodiments, a solution comprising benzyl alcohol and budiodarone for reducing atrial fibrillation burden in a patient. The present disclosure provides, in some embodiments, a solution comprising benzyl alcohol and budiodarone for reducing atrial fibrillation burden in a patient. The present disclosure provides, in some embodiments, a solution comprising benzyl alcohol and budiodarone for preventing atrial remodeling in a patient. The present disclosure provides, in some embodiments, a solution comprising benzyl alcohol and budiodarone for reversing atrial remodeling in a patient. The present disclosure provides, in some embodiments, a solution comprising benzyl alcohol and budiodarone for reducing stroke rate in a patient. The present disclosure provides, in some embodiments, a solution comprising benzyl alcohol and budiodarone for increasing time in normal sinus rhythm in a patient. The present disclosure provides, in some embodiments, a solution comprising benzyl alcohol and budiodarone for treating a refractory atrial fibrillation patient comprising administering to patient refractory to one or more anti-arrhythmic drugs.

The present disclosure provides, in some embodiments, a solution comprising benzyl alcohol and budiodarone for the manufacture of a medicament for treating cardiac arrhythmia or reducing the incidence of cardiac arrhythmias. The present disclosure provides, in some embodiments, a solution comprising benzyl alcohol and budiodarone for the manufacture of a medicament for restoring sinus and ventricular rhythms in a patient having persistent atrial fibrillation, paroxysmal atrial fibrillation, atrial flutter, ventricular arrhythmias, or ventricular fibrillation. The present disclosure provides, in some embodiments, a solution comprising benzyl alcohol and budiodarone for the manufacture of a medicament for restoring normal cardiac rhythms in a patient. The present disclosure provides, in some embodiments, a solution comprising benzyl alcohol and budiodarone for the manufacture of a medicament for reducing atrial fibrillation burden in a patient. The present disclosure provides, in some embodiments, a solution comprising benzyl alcohol and budiodarone for the manufacture of a medicament for reducing atrial fibrillation burden in a patient. The present disclosure provides, in some embodiments, a solution comprising benzyl alcohol and budiodarone for the manufacture of a medicament for preventing atrial remodeling in a patient. The present disclosure provides, in some embodiments, a solution comprising benzyl alcohol and budiodarone for the manufacture of a medicament for reversing atrial remodeling in a patient. The present disclosure provides, in some embodiments, a solution comprising benzyl alcohol and budiodarone for the manufacture of a medicament for reducing stroke rate in a patient. The present disclosure provides, in some embodiments, a solution comprising benzyl alcohol and budiodarone for the manufacture of a medicament for increasing time in normal sinus rhythm in a patient. The present disclosure provides, in some embodiments, a solution comprising benzyl alcohol and budiodarone for the manufacture of a medicament for treating a refractory atrial fibrillation patient comprising administering to patient refractory to one or more anti-arrhythmic drugs.

Additional embodiments, features, and advantages of the invention will be apparent from the following detailed description and through practice of the invention.

For the sake of brevity, the disclosures of the publications cited in this specification, including patents, are herein incorporated by reference in their entirety.

### BRIEF DESCRIPTION OF THE DRAWINGS

FIG. 1 shows a graph of the amounts of solubilized budiodarone tartrate with various concentrations of benzyl alcohol.

FIG. 2 shows a graph of resulting molar ratios between benzyl alcohol (BnOH) and budiodarone tartrate in solution with various concentrations of benzyl alcohol.

FIG. 3 shows a graph of amounts of budiodarone tartrate with various concentrations of co-solvents.

FIG. 4 shows a graph of resulting molar ratios between benzyl alcohol and over three budiodarone concentrations.

FIG. 5 shows ¹H-NMR spectrum of benzyl alcohol and budiodarone tartrate in a 3.04 molar ratio.

FIG. 6 shows a graph of amounts of solubilized amiodarone hydrochloride with various concentrations of benzyl alcohol (BnOH).

FIG. 7 shows a graph of resulting molar ratios between benzyl alcohol (BnOH) and amiodarone (amio) with various concentrations of benzyl alcohol.

FIG. 8 shows a graph of the volume of the hyperconcentrated solutions as a function of the volume of water added to the solution of benzyl alcohol and budiodarone at 3/1 molar ratio.

FIG. 9 shows a graph of budiodarone concentration in the hyperconcentrated solution as a function of the volume of water added to the solution of benzyl alcohol and budiodarone at 3:1 molar ratio.

FIG. 10 shows a dose-dependent decrease in atrial fibrillation burden (AFB) on budiodarone, with overall burden decreasing 54% on 400 mg BID and 75% on 600 mg BID. AFB is the duration of time a subject's cardiac rhythm was AF divided by the total time recorded for the study period, expressed as a percent. FIG. 10 is adapted from US Publication No. 2011/0136779.

FIG. 11 shows the reduction of AFB on budiodarone, as well as a return to pre-treatment burden state after washout. FIG. 11 is adapted from US Publication No. 2011/0136779.

FIG. 12 shows the median percent change from baseline for three parameters on 600 mg BID budiodarone. Top left graph: number of AT/AF episodes decrease by more than 60% in all three treatment months. Top right: duration of episodes decreases by more than 50% in treatment month 1, followed by a decrease of more than 70% in treatment month 2 and nearly 70% in treatment month 3. FIG. 12 is adapted from US Publication No. 2011/0136779.

FIG. 13 shows the duration of AF episodes at baseline, after treatment months 1, 2 and 3, and after washout for placebo and for 200, 400 and 600 mg BID of budiodarone. FIG. 13 is adapted from US Publication No. 2011/0136779.

FIG. 14 shows the median duration of AF episodes in hours for placebo and budiodarone at 200, 400 and 600 mg BID at baseline, after treatment months 1, 2 and 3, and after washout. FIG. 14 is adapted from US Publication No. 2011/0136779.

FIG. 15 shows the cumulative time of all episodes over 24 hours for placebo and budiodarone at 200, 400 and 600 mg BID at baseline, after treatment months 1-3, and after washout. FIG. 15 is adapted from US Publication No. 2011/0136779.

FIG. 16 shows the results of a Phase 2 pilot study of budiodarone. Mean absolute AFB, relative reductions in burden and changes in episode number and duration are described. Significant reductions in AFB and episode duration were observed with budiodarone at certain doses. FIG. 16 is adapted from US Publication No. 2011/0136779.

### DETAILED DESCRIPTION

The following terms have the following meanings unless otherwise indicated. Any undefined terms have their art recognized meanings.

The term “solubility” refers to the analytical composition of a saturated solution, expressed in terms of the proportion of a designated solute in a designated solvent. The solubility can be expressed in terms, such as a concentration, molality, mole fraction, and mole ratio.

The term “concentration” refers to an amount of substance per defined space.

A ratio is a relationship between two numbers of the same kind. A ratio is expressed as “the ratio of A to B,” “A is to B,” “A to B,” “A:B,” “A/B,” or a fraction that is the quotient of A divided by B.

The term “% v/v” refers to the ratio of the volume of the named component to the sum of the volumes of all components used to make the mixture, times 100.

The term “% w/v” refers to the mass (in grams) of a substance dissolved in or mixed with 100 milliliters of solution or mixture. Thus 1% w/v is equal to 1 gram per deciliter (g/dL) or 10 grams per liter (g/L).

The term “stable” refers to the state of maintaining integrity of a compound or composition over a period of time (e.g., during manufacture and/or storage). A lack of stability can be determined by the appearance of degradation products (e.g., via HPLC, mass spectrometry, etc.) or physical changes (e.g., appearance of particulates, cloudiness of solution, etc.) over time as compared to a fresh sample of the compound or composition.

The term “clear” refers to optical clarity. In some embodiments, a solution is clear as evidenced by a reading by UV spectroscopy at 550 nm. At 550 nm, a clear solution would have an absorption of less than 0.001 AU. Another method of determining a clear solution is by using a turbidmeter. An example is a HACH Turbidimeter (Model 2100AN, Hack Company, Loveland, Colo.), in which a reading of around 1 NTU (Nephelometric Turbidity Units) and not more than 3 NTU indicates a clear solution. When such a reading is as high as around 5 to 10 NTU, a sample is not clear, but rather slightly hazy or very slightly hazy.

The term “solvent” refers to a liquid that dissolves a solid, liquid, or gaseous solute, resulting in a solution. “Co-solvent” refers to a second, third, etc. solvent used with a primary solvent.

The term “pharmaceutically acceptable salt” means a salt which is acceptable for administration to a subject, such as a mammal (salts with counterions having acceptable mammalian safety for a given dosage regime). Such salts can be derived from pharmaceutically acceptable inorganic or organic bases and from pharmaceutically acceptable inorganic or organic acids. “Pharmaceutically acceptable salt” refers to pharmaceutically acceptable salts of a compound, which salts are derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, formate, tartrate, besylate, mesylate, acetate, maleate, oxalate, and the like.

The term “salt thereof” means a compound formed when a proton of an acid is replaced by a cation, such as a metal cation or an organic cation and the like. Where applicable, the salt is a pharmaceutically acceptable salt, although this is not required for salts of intermediate compounds that are not intended for administration to the subject. By way of example, salts of the present compounds include those wherein the compound is protonated by an inorganic or organic acid to form a cation, with the conjugate base of the inorganic or organic acid as the anionic component of the salt.

It will be appreciated that the term “or a salt or stereoisomer thereof” is intended to include all permutations of salts and stereoisomers, such as a pharmaceutically acceptable salt of a stereoisomer of subject compound.

As used herein, an “effective dosage” or “effective amount” of drug, compound, or pharmaceutical composition is an amount sufficient to effect beneficial or desired results. An effective dosage can be administered in one or more administrations. As is understood in the clinical context, an effective dosage of a drug, compound, or pharmaceutical composition may or may not be achieved in conjunction with another drug, compound, or pharmaceutical composition. Thus, an “effective dosage” may be considered in the context of administering one or more therapeutic agents, and a single agent may be considered to be given in an effective amount if, in conjunction with one or more other agents, a desirable result may be or is achieved.

As used herein, “in conjunction with” refers to administration of one treatment modality in addition to another treatment modality. As such, “in conjunction with” refers to administration of one treatment modality before, during or after administration of the other treatment modality to the individual.

An “individual” or a “subject” is a mammal, more preferably a human. Mammals also include, but are not limited to, farm animals, sport animals, pets (such as cats, dogs, horses), primates, mice and rats.

As used herein and in the appended claims, the singular forms “a,” “an,” and “the” include plural reference unless the context clearly indicates otherwise.

Reference to “about” a value or parameter herein includes (and describes) embodiments that are directed to that value or parameter per se. For example, description referring to “about X” includes description of “X.”

It is understood that aspect and variations of the invention described herein include “consisting” and/or “consisting essentially of” aspects and variations. The term “comprising” means that other elements can also be present in addition to the defined elements presented. The use of “comprising” indicates inclusion rather than limitation. The term “consisting essentially of” refers to those elements required for a given embodiment. The term permits the presence of elements that do not materially affect the basic and novel or functional characteristic(s) of that embodiment of the invention. The term “consisting of” refers to components as described herein, which are exclusive of any element not recited in that description of the embodiment.

The term “substantially free of” refers to compositions completely lacking the component or having such a small amount of the component that the component does not affect the effectiveness of the composition. The component may be present as an impurity or as a contaminant and shall be less than 0.5 wt. %. In another embodiment, the amount of the component is less than 0.1 wt. % and in yet another embodiment, the amount of component is less than 0.01 wt. %. In some embodiments, the amount of the component is below the limit of detection for analytical techniques routinely used in the art.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference in their entirety to disclose and describe the methods and/or materials in connection with which the publications are cited.

Except as otherwise noted, the methods and techniques of the present embodiments are generally performed according to conventional methods well known in the art and as described in various general and more specific references that are cited and discussed throughout the present specification. See, e.g., Loudon, Organic Chemistry, 4^{th} edition, New York: Oxford University Press, 2002, pp. 360-361, 1084-1085; Smith and March, March's Advanced Organic Chemistry: Reactions, Mechanisms, and Structure, 5^{th} edition, Wiley-Interscience, 2001.

The nomenclature used herein to name the subject compounds is illustrated in the Examples herein. The chemical naming protocol and structure diagrams used herein employ and rely on the chemical naming features as utilized by the ChemDraw Ultra program (Version 12.0, Cambridgesoft Corp., Cambridge, Mass.). Certain compound names were derived from the structures using the Autonom program as utilized by Chemdraw Ultra.

The present invention relates to budiodarone formulations which comprise a solution comprising benzyl alcohol and budiodarone, wherein the budiodarone has an aqueous solubility of about 200 mg/ml to about 1250 mg/ml when contacted with an aqueous solution. The budiodarone formulations can be highly concentrated and stored with stability, yet the formulations are also dilutable and can be delivered to a subject with ease and immediacy. The formulations of budiodarone and benzyl alcohol are described below.

Budiodarone

Budiodarone is an anti-arrhythmic agent that can be used for the treatment of atrial fibrillation. In some embodiments, budiodarone is budiodarone tartrate.

| | |
|---|---|
| 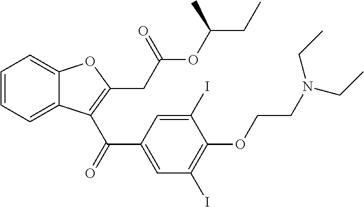 [SMILES: CC[C@H](C)OC(=O)Cc1oc2ccccc2c1C(=O)c1cc(I)c(OCCN(CC)CC)c(I)c1] | Budiodarone; 2-Benzofuranacetic acid, 3-[4-[2- (diethylamino)ethoxy]-3,5-diiodobenzoyl]-, (1S)-1-methylpropyl ester; (1S)-1-methylpropyl 2-(3-{4-[2- (diethylamino)ethoxy]-3,5- diiodobenzoyl }benzofuran-2-yl)acetate |
| 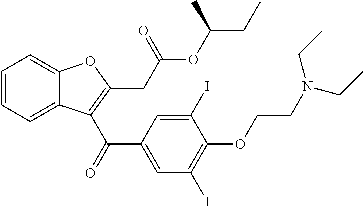 [SMILES: CC[C@H](C)OC(=O)Cc1oc2ccccc2c1C(=O)c1cc(I)c(OCCN(CC)CC)c(I)c1] [HOOC—CH(OH)]₂ | Budiodarone tartrate; 2-Benzofuranacetic acid, 3-[4-[2- (diethylamino)ethoxy]-3,5-diiodobenzoyl]-, (1S)-1-methylpropyl ester, (2R,3R)-2,3- dihydroxybutanedioate (1:1); (1S)-1-methylpropyl 2-(3-{4-[2- (diethylamino)ethoxy]-3,5- diiodobenzoyl}benzofuran-2-yl)acetate, hydrogen (2R,3R)-2,3-dihydroxybutanedioate |

Budiodarone, when taken orally, significantly reduces atrial-fibrillation burden in a dose-dependent manner and is safe and well tolerated at even the highest doses studied of 600 mg twice daily (for a total daily dose of 1200 mg).

Benzyl Alcohol

Benzyl alcohol (BnOH) has been widely used in parenteral formulations (see for example Niazi, S. K. 2004. Handbook of Pharmaceutical Manufacturing Formulations: Volume 3 Liquid Products, CRC Press, Boca Raton, Fla. And Niazi, S. K. 2004. Handbook of Pharmaceutical Manufacturing Formulations Volume 50-ver-the-Counter Products, CRC Press, Boca Raton, Fla.). Benzyl alcohol is also widely used as a preservative in foods and in cosmetics. Although a relatively safe molecule to administer to subjects, it is nevertheless associated with certain toxicity and safe limits have been recommended. For example, benzyl alcohol is not recommended for use in neonates where deaths due to benzyl alcohol have been reported. On the basis of animal studies, it has been estimated that rapid intravenous infusion of adult humans with as much as 30 ml of 0.9% benzyl alcohol (approximately 4.5 mg/kg) in saline should be safe (Kimura E T, Darby T D, Krause R A, Brondyk H D “Parenteral toxicity studies with benzyl alcohol” Toxicol. Appl. Pharmacol. 1971; 18:60). The World Health Organization (WHO) has set safe limits at 5 mg/kg. Therefore, it is desirable to minimize the amount of benzyl alcohol in formulations to the greatest extent compatible with practical use.

Solution of Budiodarone and Benzyl Alcohol

Budiodarone tartrate has a low aqueous solubility of about 7 mg/ml at room temperature. However, it was found surprisingly that a solution comprising benzyl alcohol and budiodarone, wherein the budiodarone has an aqueous solubility of about 200 mg/ml to about 1250 mg/ml when contacted with an aqueous solution can be prepared.

It was found that budiodarone, when mixed with a small amount of benzyl alcohol in water at room temperature (about 22° C.), can form a molecular association complex in a ratio of about 5 molecules of benzyl alcohol for 1 molecule of budiodarone and which is water soluble over a surprisingly wide range of concentrations (e.g. about 200 mg/ml to about 1250 mg/ml). In other words, when benzyl alcohol is added to a suspension of budiodarone in water and the components are mixed together, the two molecules associate in a 5 to 1 ratio and a clear solution forms. With benzyl alcohol, the aqueous solubility of budiodarone can be improved, even at other ratios. For example, solutions comprising benzyl alcohol and budiodarone of up to 11 to 1 ratio of benzyl alcohol to budiodarone have improved aqueous solubility of budiodarone.

The ability to use these molecular association complexes of benzyl alcohol and budiodarone to achieve higher concentration solutions of budiodarone means that clinically relevant doses of budiodarone may be administered both intravenously and orally in forms (e.g., gelcaps) and amounts (e.g., intravenous infusions) that are more convenient and palatable to individuals, thereby increasing the rate of patient compliance, reducing the costs associated with drug production, transport and storage, and the ease of reconstitution of injectable preparations freshly before use in patients. Being able to achieve much higher concentrations of budiodarone in solution will also enable hospital personnel to inject high concentrations of the drug, thereby allowing for much higher blood levels (within safety limits) and a better chance to terminate arrhythmias.

It was found that solutions comprising benzyl alcohol and budiodarone with ratios of less than 5 to 1 of benzyl alcohol to budiodarone also have improved aqueous solubility of budiodarone. For example, if benzyl alcohol and budiodarone were pre-mixed at a ratio of less than 5 to 1 of benzyl alcohol to budiodarone, the solution formed from pre-mixing has an aqueous solubility of about 200 mg/ml to about 1250 mg/ml when contacted with an aqueous solution.

The present disclosure provides a solution comprising benzyl alcohol and budiodarone, wherein the budiodarone has an aqueous solubility of about 200 mg/ml to about 1250 mg/ml when contacted with an aqueous solution can be prepared. As used herein, contact with an aqueous solution refers to the presence of the solution comprising benzyl alcohol and budiodarone in water or an aqueous vehicle.

In some embodiments, the aqueous solubility is measured at room temperature, such as about 18-20, 20-22, 22-24, or 24-26° C. Room temperature includes 18, 19, 20, 21, 22, 23, 24, 25, and 26° C. In some embodiments, the solution comprising benzyl alcohol and budiodarone is clear or substantially free of particulates.

In some embodiments, the budiodarone has an aqueous solubility of about 200 mg/ml to about 750 mg/ml when contacted with an aqueous solution. In some embodiments, the budiodarone has an aqueous solubility of about 750 mg/ml to about 1250 mg/ml when contacted with an aqueous solution. In some embodiments, the budiodarone has an aqueous solubility of about 200-300, 300-400, 400-500, 500-600, 600-700, 700-800, 800-900, 900-1000, 1000-1100, 1100-1200, or 1200-1250 mg/ml when contacted with an aqueous solution. In some embodiments, the budiodarone has an aqueous solubility of about 200, 300, 400, 500, 600, 700, 800, 900, 1000, 1100, or 1200 mg/ml when contacted with an aqueous solution.

In some embodiments, budiodarone solutions have concentrations of about 200 mg/ml to about 750 mg/ml when contacted with an aqueous solution. In some embodiments, budiodarone solutions have concentrations of about 750 mg/ml to about 1250 mg/ml when contacted with an aqueous solution. In some embodiments, budiodarone solutions have concentrations of about 200-300, 300-400, 400-500, 500-600, 600-700, 700-800, 800-900, 900-1000, 1000-1100, 1100-1200, or 1200-1250 mg/ml when contacted with an aqueous solution. In some embodiments, budiodarone solutions have concentrations of about 200, 300, 400, 500, 600, 700, 800, 900, 1000, 1100, or 1200 mg/ml when contacted with an aqueous solution.

In some embodiments, the solution comprising benzyl alcohol and budiodarone can comprise water. The present disclosure also provides an aqueous solution comprising benzyl alcohol and budiodarone, wherein the budiodarone is present in the solution in a concentration of about 200 mg/ml to about 1250 mg/ml. In some embodiments, the concentration is measured at room temperature, such as about 18-20, 20-22, 22-24, or 24-26° C. Room temperature includes 18, 19, 20, 21, 22, 23, 24, 25, and 26° C. In some embodiments, the aqueous solution comprising benzyl alcohol and budiodarone is clear or substantially free of particulates.

In some embodiments, the budiodarone is present in the aqueous solution in a concentration of about 200 mg/ml to about 750 mg/ml. In some embodiments, the budiodarone is present in the aqueous solution in a concentration of about 750 mg/ml to about 1250 mg/ml. In some embodiments, the budiodarone is present in the aqueous solution in a concentration of about 200-300, 300-400, 400-500, 500-600, 600-700, 700-800, 800-900, 900-1000, 1000-1100, 1100-1200, or 1200-1250 mg/ml. In some embodiments, the budiodarone is present in the aqueous solution in a concentration of about 200, 300, 400, 500, 600, 700, 800, 900, 1000, 1100, or 1200 mg/ml.

The present disclosure provides a solution comprising benzyl alcohol and budiodarone, wherein the benzyl alcohol and budiodarone are present in a molar ratio of benzyl alcohol to budiodarone of between about 0.5 to 1 and 11 to 1. In some embodiments, the benzyl alcohol and budiodarone are present in a molar ratio of benzyl alcohol to budiodarone of about 0.5 to 1; 1 to 1; 1.5 to 1; 2 to 1; 2.5 to 1; 3 to 1; 3.5 to 1; 4 to 1; 4.5 to 1; 5 to 1; 5.5 to 1; 6 to 1; 6.5 to 1; 7 to 1; 7.5 to 1; 8 to 1; 8.5 to 1; 9 to 1; 9.5 to 1; 10 to 1; 10.5 to 1; or 11 to 1.

In some embodiments, the present disclosure provides a solution comprising benzyl alcohol and budiodarone, wherein the benzyl alcohol and budiodarone are present in a molar ratio of benzyl alcohol to budiodarone of between about 0.5 to 1 and 4.5 to 1. In some embodiments, the benzyl alcohol and budiodarone are present in a molar ratio of benzyl alcohol to budiodarone of between about 2 to 1 and 4 to 1. In some embodiments, the benzyl alcohol and budiodarone are present in a molar ratio of benzyl alcohol to budiodarone of about 3 to 1.

In some embodiments, the present disclosure provides a solution comprising benzyl alcohol and budiodarone, wherein the benzyl alcohol and budiodarone are present in a molar ratio of benzyl alcohol to budiodarone of between about 5 to 1 and 11 to 1. In some embodiments, the benzyl alcohol and budiodarone are present in a molar ratio of benzyl alcohol to budiodarone of about 5 to 1.

In some embodiments, the solution comprising benzyl alcohol and budiodarone can comprise other co-solvents which do not interfere with the high aqueous solubility of the benzyl alcohol/budiodarone solution but which may be added for the purpose of increasing shelf-life, increasing palatability, or increasing the ease of formulation when used in a large-scale facility. Such co-solvents would include emulsifiers such as labrasol, diluents such as ethanol, or a highly-concentrated sugar solution (syrup). In some embodiments, a tonicity adjuster can be added to adjust the tonicity of the formulation. Examples of tonicity adjusters include salts, such as sodium chloride, potassium chloride, mannitol and glycerin. These are only given as examples and pharmaceutical formulation scientists would easily recognize the benefits of other co-solvents for the safe administration of the benzyl alcohol/budiodarone solution to man.

Pharmaceutical Compositions and Forms

For treatment purposes, a pharmaceutical composition according to the invention comprises a solution of benzyl alcohol and budiodarone. The pharmaceutical compositions may further comprise one or more pharmaceutically-acceptable excipients. A pharmaceutically-acceptable excipient is a substance that is non-toxic and otherwise biologically suitable for administration to a subject. Such excipients facilitate administration of the compounds described herein and are compatible with the active ingredient. Examples of pharmaceutically-acceptable excipients include stabilizers, lubricants, surfactants, diluents, anti-oxidants, binders, coloring agents, bulking agents, emulsifiers, or taste-modifying agents. In some embodiments, pharmaceutical compositions according to the embodiments are sterile compositions. Pharmaceutical compositions may be prepared using compounding techniques known or that become available to those skilled in the art.

Sterile compositions are also contemplated by the embodiments, including compositions that are in accord with national and local regulations governing such compositions.

The pharmaceutical compositions described herein may be formulated as solutions, emulsions, suspensions, dispersions, or inclusion complexes such as cyclodextrins in suitable pharmaceutical solvents or carriers, or as pills, tablets, lozenges, suppositories, sachets, dragees, granules, powders, powders for reconstitution, or capsules along with solid carriers according to conventional methods known in the art for preparation of various dosage forms. Pharmaceutical compositions of the embodiments may be administered by a suitable route of delivery, such as oral, parenteral, rectal, nasal, topical, or ocular routes, or by inhalation. Preferably, the compositions are formulated for intravenous, oral, or rectal administration.

Pharmaceutical compositions that are intended for absorption through the intestinal barrier can also incorporate various excipients that are commonly used in these compositions, such as diluents, glidants, lubricants, disintegrants, and wetting agents. These various excipients are used to improve handling and reproducibility of the dosage form by industrial formulation machines and are not necessary to improve the bioavailability of the solution comprising benzyl alcohol and budiodarone (see for example, L. Lachman, H. A. Lieberman, J. L. Kanig (1986). The Theory and Practice of Industrial Pharmacy (3^{rd} ed.). Lea & Febiger, Philadelphia.)

In some embodiments, the pharmaceutical composition comprises an emulsifier, such as labrasol, gelucire, phospholipid, cholesterol, and cellulose ester. In some embodiments, the pharmaceutical composition comprises an emulsifier, such as labrasol, gelucire, phospholipid, cholesterol, and plant sterols.

In some embodiments, the pharmaceutical composition is substantially free of a solubilizing excipient. A solubilizing excipient is agent used to increase solubility of a substance. Solubilization is taken to mean an improvement in the solubility by virtue of the solubilizing excipient that can convert substances that are insoluble or virtually insoluble in water into clear, or opalescent, aqueous solutions without changing the chemical structure of these substances in the process. In some embodiments, the pharmaceutical composition is substantially free of a solubilizing excipient selected from water-soluble organic solvents (polyethylene glycol 300, polyethylene glycol 400, ethanol, propylene glycol, glycerin, N-methyl-2-pyrrolidone, dimethylacetamide, dimethylsulfoxide, etc.), non-ionic surfactants (CREMOPHOR® EL, CREMOPHOR® RH 60, polysorbate 80, SOLUTOL® HS 15, etc.), water-insoluble lipids, phospholipids (usually for injectable emulsions), and various cyclodextrins. In some embodiments, the pharmaceutical composition is substantially free of TWEEN.

Depending on the amount of aqueous vehicle added to the solution comprising benzyl alcohol and budiodarone, benzyl alcohol is at a concentration of between about 0.005 and 48.5% v/v. In some embodiments, benzyl alcohol is at a concentration of about 1.5-48.5% v/v; 4-48.5% v/v; 15-48.5% v/v; or 18.5-48.5% v/v. In some embodiments, benzyl alcohol is at a concentration of about 0.5-18.5% v/v; 0.5-15% v/v; or 0.5-10% v/v. In some embodiments, benzyl alcohol is at a concentration of about 0.25-4.5% v/v. In some embodiments, an aqueous solution comprising benzyl alcohol and budiodarone can comprise about 0.25, 0.5, 1, 1.5, 2, 2.5, 3, 3.5, 4, or 4.5% v/v of benzyl alcohol. In some embodiments, an aqueous solution comprising benzyl alcohol and budiodarone can comprise about 5, 6, 7, 8, 9, 10, 11, 12, 13, 14, 15, 16, 17, 18, 19 or 20% v/v of benzyl alcohol.

In some embodiments, a solution suitable for administration contains budiodarone at a concentration of between about 0.01 and 1250 mg/ml. In some embodiments, the budiodarone is present in the aqueous solution in a concentration of about 0.01-50, 50-100, 100-200, 200-300, 300-400, 400-500, 500-600, 600-700, 700-800, 800-900, 900-1000, 1000-1100, 1100-1200, or 1200-1250 mg/ml. In some embodiments, the budiodarone is present in the aqueous solution in a concentration of about 0.01, 0.05, 0.1, 1, 5, 10, 25, 50, 100, 200, 300, 400, 500, 600, 700, 800, 900, 1000, 1100, or 1200 mg/ml. In some embodiments, the budiodarone is present in the solution in a concentration of about 0.01, 0.05, 0.1, 1, 5, 10, 20, 30, 40, 50, 60, 70, 80, 90, 100, 125, 150, 175, 200, 225, 250, 275, 300, 325, 350, 375, 400, 425, 450, 475, or 500 mg/ml. In some embodiments, the budiodarone is present in the aqueous solution in a concentration of about 600, 700, 800, 900, 1000, 1100, or 1200 mg/ml.

In some embodiments, as a pharmaceutical composition, a solution contains benzyl alcohol and budiodarone at a molar ratio of benzyl alcohol to budiodarone of between about 1 to 1 and 11 to 1, or between about 3 to 1 and 5 to 1, or about 3 to 1. In some embodiments, the benzyl alcohol and budiodarone are in a molar ratio of benzyl alcohol to budiodarone is about 1 to 1; 1.5 to 1; 2 to 1; 2.5 to 1; 3 to 1; 3.5 to 1; 4 to 1; 4.5 to 1; 5 to 1; 5.5 to 1; 6 to 1; 6.5 to 1; 7 to 1; 7.5 to 1; 8 to 1; 8.5 to 1; 9 to 1; 9.5 to 1; 10 to 1; 10.5 to 1; or 11 to 1.

Oral Administration

Due to its much higher aqueous solubility than budiodarone alone, the solution comprising benzyl alcohol and budiodarone can advantageously be administered orally. Because the budiodarone in the solution comprising benzyl alcohol and budiodarone is soluble in water, the budiodarone is absorbed faster through the intestinal epithelium, resulting in a more desirable pharmacokinetic profile. In some embodiments, an aqueous solution comprising benzyl alcohol and budiodarone may be provided in an oral form.

For oral administration, the solution comprising benzyl alcohol and budiodarone may be provided in a solid form, such as a capsule, or as a solution, emulsion, or suspension. In some embodiments, the solid form of oral administration can comprise pharmaceutically acceptable excipients. Capsules for oral administration include hard and soft gelatin capsules. Hard gelatin capsules can be obtain commercially (Capsulgel, Greenwood N.C.; Qualicaps, Whitsett, N.C.; ACG capsules, South Plainfield, N.J.) Hard gelatin capsules can be made from HPMC, and bovine, fish and vegetable sources. To prepare hard gelatin capsules, the active ingredient may be mixed with a solid or semi-solid diluent; or can be inserted into the hard capsule as a lyophilized powder. To prepare soft gelatin capsules, the active ingredient as a solution can be injected into the soft gelatin capsule. (B E Jones and F Podczeck “Pharmaceutical Capsules,” 2^{nd} edition, Pharmaceutical Press, London, 2004)

In some embodiments, the amount of budiodarone present in a capsule can be between about 50 and 800 mg. In some embodiments, the amount of budiodarone present in a capsule can be about 50-100, 100-200, 200-300, 300-400, 400-500, 500-600, 600-700, or 700-800 mg. In some embodiments, the amount of budiodarone present in capsule can be about 100, 150, 200, 250, 300, 350, 400, 450, 500, 550, 600, 650, 700, 750, or 800 mg.

The solution comprising benzyl alcohol and budiodarone may be provided in a syrup form. Since glucose is compatible with an aqueous solution of benzyl alcohol and budiodarone, the solution comprising benzyl alcohol and budiodarone can be mixed with glucose and can be made into a syrup form to be taken by mouth. In some embodiments, the syrup can comprise pharmaceutically acceptable excipients. In some embodiments, the concentration of budiodarone into the syrup can be between about 10 and 100 mg/ml. In some embodiments, the amount of budiodarone present in the syrup can be about 10-20, 20-30, 30-40, 40-50, 50-60, 60-70, 70-80, 80-90, or 90-100 mg/ml.

In some embodiments, the solution comprising benzyl alcohol and budiodarone for oral administration can be mixed with various excipients ordinarily used in pharmaceutical delivery of oral drugs. It can be made into a slow-release formulation or into an immediate-release formulation. Mixing of the solution comprising benzyl alcohol and budiodarone with an excipient can be aided by the use of co-solvents. Low-boiling co-solvents can be used to dissolve budiodarone, benzyl alcohol, and the excipient in order to make a homogeneous mixture. The co-solvent is then evaporated and then is left under high vacuum to remove the co-solvent. For reasons of loss of benzyl alcohol under high vacuum, it is practical to start from an excess of benzyl alcohol and to monitor the mixture until the desired ratio is obtained. Typical excipients that can be used to formulate the pharmaceutical composition are hydroxypropylmethyl celluloses (HPMC) of various grades (high viscosity vs low viscosity). These HPMC have been extensively used in the preparation of immediate-release and slow-release formulations. Other excipients include polyvinylpyrrolidone (Povidone); microcrystalline cellulose; lactose; magnesium stearate; long-chain and medium-chain triglycerides, emulsifiers, and the like.

In some embodiments, the solution comprising benzyl alcohol and budiodarone can also be mixed with inert fillers such as cellulose esters, microcrystalline cellulose, or other excipients commonly used in solid oral dosage form preparations in order to make a solid dosage form that can be compressed into a pellet. The unit doses of budiodarone in these pellets will be between 50 mg and 800 mg.

Injectable Administration

The solution comprising benzyl alcohol and budiodarone may be provided in an injectable form. A solution comprising benzyl alcohol and budiodarone has an aqueous solubility of the budiodarone of about 200 mg/ml to about 1250 mg/ml when contacted with an aqueous solution. In some embodiments, an aqueous solution comprising benzyl alcohol and budiodarone may be provided in an injectable form. In some embodiments, the injectable form is intravenous infusion. In some embodiments, the injectable form is bolus injection. In some embodiments, the injectable form is intra-muscular injection, subcutaneous injection, or intraperitoneal injection.

The solution can be formulated for injection by diluting the solution with the desired volume of an aqueous vehicle for injection. In some embodiments, the aqueous vehicle is an isotonic aqueous solvent. In some embodiments, the isotonic aqueous solvent is not ionic. For example, the addition of certain ionic solutions, such as solutions containing sodium chloride or various inorganic or organic salts and pH buffers can result in a precipitate of budiodarone. Suitable non-ionic aqueous diluents include, but are not limited to water, dextrose solution, and sucrose solution. In some embodiments, the solvent for dilution of the solution comprising benzyl alcohol and budiodarone is selected from water, dextrose solution, and sucrose solution. In some embodiments, water is deionized. In some embodiments, the dextrose solution is 1, 2, 3, 4, 5, 6, 7, 8, 9 or 10% w/v. In some embodiments, the sucrose solution is 1, 2, 3, 4, 5, 6, 7, 8, 9 or 10% w/v.

The composition of an aqueous solution may also comprise a non-ionic solute at a concentration of between 4 and 6 g/100 ml. In some embodiments, the non-ionic solute is at a concentration of about 5 g/100 ml. In some embodiments, the non-ionic solute is sucrose or dextrose.

An osmolality modifier can also be added to an aqueous solution comprising benzyl alcohol and budiodarone. In some embodiments, an osmolality modifier is a non-dissociated small molecule, such as dimethylacetamide, propylene glycol, or polyethylene glycol. The osmolality modifier can be added to an aqueous solution comprising benzyl alcohol and budiodarone in the amount to maintain the osmolality of the solution close to the osmolality of blood when administered to a subject. In some embodiments, the osmolality modifier can maintain the osmolality of the solution within 1, 2, 3, 4, 5, or 10% of the osmolality of blood when administered to a subject. In some embodiments, the solution can have an osmolality that is twice that of blood.

In some embodiments, an aqueous solution suitable for injectable administration contains budiodarone at a concentration of between about 0.1 and 500 mg/ml. In some embodiments, an aqueous solution suitable for injectable administration contains budiodarone at a concentration of about 0.1-50, 50-100, 100-200, 200-300, or 400-500 mg/ml. In some embodiments, an aqueous solution suitable for injectable administration contains budiodarone at a concentration of about 1-500, 10-500, 50-500, 100-500, 200-500, 300-500, or 400-500 mg/ml. In some embodiments, an aqueous solution suitable for injectable administration contains budiodarone at a concentration of about 0.1-500, 0.1-400, 0.1-300, 0.1-200, 0.1-100, 0.1-50, or 0.1-10 mg/ml. In some embodiments, the budiodarone is present in the aqueous solution in a concentration of about 5, 10, 20, 30, 40, 50, 60, 70, 80, 90, 100, 125, 150, 175, 200, 225, 250, 275, 300, 325, 350, 375, 400, 425, 450, 475, or 500 mg/ml. In some embodiments, the budiodarone is present in the aqueous solution in a concentration of about 50, 100, 150, 200, 250, 300, 350, 400, 450, or 500 mg/ml. In some embodiments, the budiodarone is present in the aqueous solution in a concentration of about 0.1, 0.5, 1, 2, 3, 4, 5, 10, 15, 20, 25, 30, 35, 40, 45, or 50 mg/ml. It is understood by health practitioners that the highest budiodarone concentrations (>50 mg/ml) should be reserved for specific applications such as depot injection (intra-muscular or sub-cutaneous). High aqueous concentrations of budiodarone can in theory be injected intravenously, but safety concerns dictate that the lowest concentrations (<50 mg/ml) be reserved for intravenous use.

Rectal Administration

The pharmaceutical compositions may be formulated in rectal compositions such as enemas, rectal gels, rectal foams, rectal aerosols, suppositories, jelly suppositories, or retention enemas, containing conventional suppository bases such as cocoa butter or other glycerides, as well as synthetic polymers such as polyvinylpyrrolidone, PEG, and the like. In suppository forms of the compositions, a low-melting wax such as, but not limited to, a mixture of fatty acid glycerides, optionally in combination with cocoa butter is first melted.

Hyperconcentrated Solution

In some embodiments, the present disclosure provides a hyperconcentrated aqueous solution of the benzyl alcohol and budiodarone. A hyperconcentrated aqueous solution of the benzyl alcohol and budiodarone comprises a concentration of budiodarone of equal to or greater than 500 mg/ml, 800 mg/ml, 900 mg/ml, 1000 mg/ml, 1100 mg/ml, or 1200 mg/ml. In some embodiments, a hyperconcentrated aqueous solution contains budiodarone at a concentration of 500-600, 600-700, 700-800, 800-900, 900-1000, 1000-1100, 1100-1200, or 1200-1250 mg/ml. In some embodiments, a hyperconcentrated aqueous solution contains budiodarone at a concentration of 500-1250, 500-1200, 500-1100, 500-1000, 500-900, 500-800, 500-700, or 500-600 mg/ml. In some embodiments, a hyperconcentrated aqueous solution contains budiodarone at a concentration of 500-1250, 600-1250, 700-1250, 800-1250, 900-1250, 1000-1250, 1100-1250, or 1200-1250 mg/ml. In some embodiments, the budiodarone is present in the hyperconcentrated aqueous solution in a concentration of about 500, 550, 600, 650, 700, 750, 800, 850, 900, 950, 1000, 1050, 1100, 1150, 1200, or 1250 mg/ml. In some embodiments, the budiodarone is present in the hyperconcentrated aqueous solution in a concentration of about 800, 850, 900, 950, 1000, 1050, 1100, 1150, 1200, or 1250 mg/ml.

A hyperconcentrated aqueous solution of the benzyl alcohol and budiodarone provides advantages, such as providing an adjustable amount budiodarone for administration to a subject. In some instances, the hyperconcentrated aqueous solution can be adjusted to an appropriate dose of budiodarone with dilution and then administered to a subject such that high blood levels of budiodarone can be achieved using a single bolus injection. In some embodiments, the high blood level of budiodarone can be achieved effectively in emergency situations, such as for the purpose of terminating cardiac arrhythmias.

In some embodiments, the hyperconcentrated aqueous solution can be diluted to a more appropriate concentration with an aqueous vehicle before injection into a subject.

In some embodiments, the hyperconcentrated aqueous solution can be provided in an oral form. In some instances, the hyperconcentrated aqueous solution is provided in a solid dosage form, such as a capsule, or as a solution, emulsion, or suspension, as discussed above. In some embodiments, the capsule is engineered to receive aqueous solutions. In some instances, the hyperconcentrated aqueous solution is provided in a capsule, such as soft gelatin capsule.

Chemical and Physical Stability of Solution of Budiodarone and Benzyl Alcohol

Physical stability of budiodarone and benzyl alcohol in aqueous solution, i.e., the budiodarone and benzyl alcohol stays in solution and does not precipitate over certain length of time, can be measured either by light scattering techniques (photon correlation spectroscopy) which measures the size of a complex of budiodarone and benzyl alcohol, or by measuring turbidity at 550 nm, which also, indirectly, is a measure of the size of the complex. A change of these parameters over time will indicate a change in the size of the molecular complex, i.e., the complex is not stable.

The chemical stability of budiodarone in a solution or aqueous solution can be evaluated by a HPLC/MS method discussed below. The identification of budiodarone on HPLC/MS can be determined by distinct peaks which can be monitored over time to determine the chemical stability.

In some embodiments, the solution of benzyl alcohol and budiodarone can be stable and stored for at least 1, 5, 10, 15, 20, 25, 30, 31, 32, 33, 34, 35, 36, 37, 38, 39, 40, 50, 60, 70, 80, or 90 days at room temperature. In some embodiments, the solution of benzyl alcohol and budiodarone can be stable and stored for at least 4, 5, 6, 7, 8, 9, 10, 11, 12, 13, 14, 15, 16, 17, 18, 19, 20, 21, 22, 23, or 24 months at room temperature. In some embodiments, the solution of benzyl alcohol and budiodarone can be stable and stored for at least 1, 2, 3, 4, 5, 6, 7, 8, 9, or 10 years at room temperature. In some embodiments, the solution of benzyl alcohol and budiodarone can be stable and stored for at least 36 days at room temperature.

In some embodiments, the hyperconcentrated aqueous solution or aqueous solution comprising benzyl alcohol and budiodarone can be stable and stored for at least 1, 5, 6, 7, 8, 9, 10, 11, 12, 13, 14, 15, 16, 17, 18, 19, 20, 25, 30, 35, 40, 50, 60, 70, 80, or 90 days at room temperature. In some embodiments, the hyperconcentrated aqueous solution or aqueous solution comprising benzyl alcohol and budiodarone can be stable and stored for at least 4, 5, 6, 7, 8, 9, 10, 11, 12, 13, 14, 15, 16, 17, 18, 19, 20, 21, 22, 23, or 24 months at room temperature. In some embodiments, the hyperconcentrated aqueous solution or aqueous solution comprising benzyl alcohol and budiodarone can be stable and stored for at least 1, 2, 3, 4, 5, 6, 7, 8, 9, or 10 years at room temperature. In some embodiments, the hyperconcentrated aqueous solution or aqueous solution comprising benzyl alcohol and budiodarone can be stable and stored for at least 13 days at room temperature.

Short-term stability of an aqueous solution at room temperature is advantageous because an aqueous solution can be stored after reconstitution and be ready for use, such as in a hospital setting. In some embodiments, an aqueous solution comprising benzyl alcohol and budiodarone can be stable for at least about 1, 2, 3, 4, 5, 6, 8, 10, 12, or 18 hours or 1, 2, 3, 4, 5, 6, 7, 8, 9, 10, 11, 12, 13, 14, 15, 16, 17, 18, 19, 20, or 21 days.

Storage of Solution of Budiodarone and Benzyl Alcohol

In some embodiments, the solution comprising benzyl alcohol and budiodarone can be stored as a solution of benzyl alcohol and budiodarone in the ratios mentioned above. In some embodiments, the solution contains benzyl alcohol and budiodarone at a molar ratio of benzyl alcohol to budiodarone of between about 1 to 1 and 11 to 1, or between about 3 to 1 and 5 to 1, or about 3 to 1. In some embodiments, the benzyl alcohol and budiodarone are in a molar ratio of benzyl alcohol to budiodarone is about 1 to 1; 1.5 to 1; 2 to 1; 2.5 to 1; 3 to 1; 3.5 to 1; 4 to 1; 4.5 to 1; 5 to 1; 5.5 to 1; 6 to 1; 6.5 to 1; 7 to 1; 7.5 to 1; 8 to 1; 8.5 to 1; 9 to 1; 9.5 to 1; 10 to 1; 10.5 to 1; or 11 to 1.

In some embodiments, the solution comprising benzyl alcohol and budiodarone can be stored for at least 1-5, 5-9, 10-14, 15-19, 20-24, 25-29, 30-34, 35-39, 40-60, or 60-90 days at room temperature or up to around 40° C. In some embodiments, the solution comprising benzyl alcohol and budiodarone can be stored for at least 1, 5, 10, 15, 20, 25, 30, 31, 32, 33, 34, 35, 36, 37, 38, 39, 40, 50, 60, 70, 80, or 90 days at room temperature or up to around 40° C. In some embodiments, the solution comprising benzyl alcohol and budiodarone can be stored for at least 4, 5, 6, 7, 8, 9, 10, 11, 12, 13, 14, 15, 16, 17, 18, 19, 20, 21, 22, 23, or 24 months at room temperature or up to around 40° C. In some embodiments, the solution comprising benzyl alcohol and budiodarone can be stored for at least 1, 2, 3, 4, 5, 6, 7, 8, 9, or 10 years at room temperature or up to around 40° C. In some embodiments, the solution comprising benzyl alcohol and budiodarone can be stored for at least 1 or 2 years at room temperature or up to around 40° C. In some embodiments, the solution comprising benzyl alcohol and budiodarone can be stored for at least 2 years at room temperature or up to around 40° C.

Room temperature includes temperature of about 18-20, 20-22, 22-24, or 24-26° C., such as 18, 19, 20, 21, 22, 23, 24, 25, and 26° C. Other suitable storage temperatures include 27, 18, 19, 30, 31, 32, 33, 34, 35, 36, 37, 38, 39, or 40° C.

The solution of benzyl alcohol and budiodarone can be stored and is stable for longer than the above time periods at cold temperatures. Cold temperature includes temperature of about 0-5, 6-10, 11-15, or 16-17° C., such as 0, 1, 2, 3, 4, 5, 6, 7, 8, 9, 10, 11, 12, 13, 14, 15, 16, or 17° C.

In some embodiments, the solution comprising benzyl alcohol and budiodarone can be stored as a hyperconcentrated aqueous solution or an aqueous solution comprising benzyl alcohol and budiodarone in the ratios mentioned above. In some embodiments, the hyperconcentrated aqueous solution or an aqueous solution contains benzyl alcohol and budiodarone at a molar ratio of benzyl alcohol to budiodarone of between about 1 to 1 and 11 to 1, or between about 3 to 1 and 5 to 1, or about 3 to 1. In some embodiments, the benzyl alcohol and budiodarone are in a molar ratio of benzyl alcohol to budiodarone is about 1 to 1; 1.5 to 1; 2 to 1; 2.5 to 1; 3 to 1; 3.5 to 1; 4 to 1; 4.5 to 1; 5 to 1; 5.5 to 1; 6 to 1; 6.5 to 1; 7 to 1; 7.5 to 1; 8 to 1; 8.5 to 1; 9 to 1; 9.5 to 1; 10 to 1; 10.5 to 1; or 11 to 1.

In some embodiments, when stored, the hyperconcentrated aqueous solution of the benzyl alcohol and budiodarone comprises a concentration of budiodarone of about 500-600, 600-700, 700-800, 800-900, 900-1000, 1000-1100, 1100-1200, or 1200-1250 mg/ml. In some embodiments, when stored, the hyperconcentrated aqueous solution of the benzyl alcohol and budiodarone comprises a concentration of budiodarone of about 500, 550, 600, 650, 700, 750, 800, 850, 900, 950, 1000, 1050, 1100, 1150, 1200, or 1250 mg/ml. In some embodiments, when stored, the aqueous solution of the benzyl alcohol and budiodarone comprises a concentration of budiodarone of about 0.1-50, 50-100, 100-200, 200-300, or 400-500 mg/ml. In some embodiments, when stored, the aqueous solution of the benzyl alcohol and budiodarone comprises a concentration of budiodarone of about 50, 100, 150, 200, 250, 300, 350, 400, 450, or 500 mg/ml.

In some embodiments, the solution comprising benzyl alcohol and budiodarone can be stored for at least 1-5, 5-9, 10-14, 15-19, 20-24, 25-29, 30-34, 35-39, 40-60, 60-90 days at room temperature or up to around 40° C. In some embodiments, the hyperconcentrated aqueous solution or aqueous solution comprising benzyl alcohol and budiodarone can be stored for at least 1, 5, 6, 7, 8, 9, 10, 11, 12, 13, 14, 15, 16, 17, 18, 19, 20, 25, 30, 35, 40, 50, 60, 70, 80, or 90 days at room temperature or up to around 40° C. In some embodiments, the hyperconcentrated aqueous solution or aqueous solution comprising benzyl alcohol and budiodarone can be stored for at least 4, 5, 6, 7, 8, 9, 10, 11, 12, 13, 14, 15, 16, 17, 18, 19, 20, 21, 22, 23, or 24 months at room temperature or up to around 40° C. In some embodiments, the hyperconcentrated aqueous solution or aqueous solution comprising benzyl alcohol and budiodarone can be stored for at least 1, 2, 3, 4, 5, 6, 7, 8, 9, or 10 years at room temperature or up to around 40° C. In some embodiments, the hyperconcentrated aqueous solution or aqueous solution can be stored for at least 1 or 2 years at room temperature or up to around 40° C. In some embodiments, the hyperconcentrated aqueous solution or aqueous solution can be stored for at least 2 years at room temperature or up to around 40° C.

Room temperature includes temperature of about 18-20, 20-22, 22-24, or 24-26° C., such as 18, 19, 20, 21, 22, 23, 24, 25, and 26° C. Other suitable storage temperatures include 27, 18, 19, 30, 31, 32, 33, 34, 35, 36, 37, 38, 39, or 40° C.

The hyperconcentrated aqueous solution or an aqueous solution comprising benzyl alcohol and budiodarone can be stored and is stable for longer than the above time periods at cold temperatures. Cold temperature includes temperature of about 0-5, 6-10, 11-15, or 16-17° C., such as 0, 1, 2, 3, 4, 5, 6, 7, 8, 9, 10, 11, 12, 13, 14, 15, 16, or 17° C.

Sterilized Solutions of Budiodarone and Benzyl Alcohol

The solution comprising benzyl alcohol and budiodarone can be sterilized by filtration sterilization. For example, filtration sterilization can be performed with 0.2 μM filters to physically remove micro-organisms in a sterile laminar-flow hood or in a sterile laminar-flow room (clean room) directly into sterile vials. Stability endpoints are visual appearance, budiodarone recovery, and pH.

The solution comprising benzyl alcohol and budiodarone is physically and chemically stable under sterile filtration conditions. Physical stability can be checked visually or by using methods such as UV turbidity assay. Chemical stability can be checked using analytical methods of quantitation such as HPLC and HPLC/MS.

If at times it becomes necessary to dilute the solution of benzyl alcohol and budiodarone before sterile filtration, then water can be subsequently removed from the resulting sterilized aqueous solution comprising benzyl alcohol and budiodarone by lyophilization to form a sterile solution of benzyl alcohol and budiodarone containing the desired amount of benzyl alcohol. The sterile solution has a long shelf life and can be reconstituted with an aqueous vehicle, such as sterile water, sterile 5% dextrose, or sterile 5% sucrose just before administration into a subject.

Process of Making Solution of Budiodarone and Benzyl Alcohol

The present disclosure provides a process for preparing a solution comprising benzyl alcohol and budiodarone in a molar ratio of benzyl alcohol to budiodarone of between about 0.5 to 1 and 4.5 to 1, wherein the budiodarone has an aqueous solubility of about 200 mg/ml to about 1250 mg/ml when contacted with an aqueous solution, and wherein the process comprises: mixing benzyl alcohol and budiodarone in a sufficient amount to make a clear solution, wherein the benzyl alcohol and budiodarone are present in a molar ratio that is between about 0.5 to 1 and 4.5 to 1.

In some embodiments, the benzyl alcohol and budiodarone are present in a molar ratio of 0.5 to 1; 1 to 1; 1.5 to 1; 2 to 1; 2.5 to 1; 3 to 1; 3.5 to 1; 4 to 1; or 4.5 to 1.

In the process, the appropriate amounts of benzyl alcohol and budiodarone are mixed. The mixing of benzyl alcohol and budiodarone can be performed at room temperature or with light heating. Light heating can be heating up to 30, 35, 40, 45, or 50° C. A clear solution refers to the optical clarity of the solution. In some embodiments, a solution is clear as evidenced by a reading by UV spectroscopy or a turbidimeter. A sufficient amount refers to a minimum amount (or more) that is used to achieve the effect. For example, a sufficient amount to make a clear solution is a minimum amount (or more) of a solvent used to make a clear solution as discussed above. The sufficient amount may be dependent on temperature.

The present disclosure also provides a process for preparing a solution comprising benzyl alcohol and budiodarone in a molar ratio of benzyl alcohol to budiodarone of between about 0.5 to 1 and 4.5 to 1, wherein the budiodarone has an aqueous solubility of about 200 mg/ml to about 1250 mg/ml when contacted with an aqueous solution, and wherein the process comprises:

(a) mixing benzyl alcohol, budiodarone, and a co-solvent in a sufficient amount to make a clear solution, wherein the benzyl alcohol and budiodarone are present in a molar ratio that is greater than between about 0.5 to 1 and 4.5 to 1; and
(b) removing the co-solvent until the solution comprises benzyl alcohol and budiodarone in a molar ratio of benzyl alcohol to budiodarone of between about 0.5 to 1 and 4.5 to 1.

In step (a) of the process, the appropriate amounts of benzyl alcohol and budiodarone are mixed with a co-solvent. In some embodiments, the benzyl alcohol and budiodarone are present in a molar ratio that is greater than between about 0.5 to 1 and 4.5 to 1, such as up to about 11 to 1. In some embodiments, for step (a), the benzyl alcohol and budiodarone are present in a molar ratio of 1 to 1; 1.5 to 1; 2 to 1; 2.5 to 1; 3 to 1; 3.5 to 1; 4 to 1; 4.5 to 1; 5 to 1; 5.5 to 1; 6 to 1; 6.5 to 1; 7 to 1; 7.5 to 1; 8 to 1; 8.5 to 1; 9 to 1; 9.5 to 1; 10 to 1; 10.5 to 1; or 11 to 1. In some embodiments, for step (a), the benzyl alcohol and budiodarone are present in a molar ratio of between about 0.5 to 1 and 11 to 1.

In some embodiments, the co-solvent has a low boiling point. In some embodiments, the co-solvent has a boiling point of less than about 205° C. Co-solvents that are suitable for the process include, but not limited to, methylene chloride, methanol, acetone, ethyl acetate, acetonitrile, and supercritical carbon dioxide. The amount of co-solvent used is an amount that would make a clear solution for the mixing step. A clear solution refers to the optical clarity of the solution. In some embodiments, a solution is clear as evidenced by a reading by UV spectroscopy or a turbidimeter. A sufficient amount refers to a minimum amount (or more) that is used to achieve the effect. For example, a sufficient amount to make a clear solution is a minimum amount (or more) of a solvent used to make a clear solution as discussed above. The sufficient amount may be dependent on temperature.

The mixing of benzyl alcohol and budiodarone can be performed at room temperature or with light heating. Light heating can be heating up to 30, 35, or 40° C.

In step (b) of the process, the co-solvent is removed with light heating or reduced pressure. In some embodiments, the co-solvent is removed at a temperature of 40° C. or greater, such as 45, 50, 55, or 60° C. In some embodiments, the co-solvent is removed with reduced pressure. Reduced pressure is a pressure that is lower than atmospheric pressure. In some embodiments, reduced pressure refers to a pressure of about 25, 30, or 35 mmHg.

The resulting solution can be left under high vacuum. High vacuum refers to a reduced pressure of about 0.5-0.9, 1-2, or 3-5 mmHg. In some embodiments, the resulting solution can be left under high vacuum for 6-12 hours, 12-24 hours, 1-2 days, 3-5 days, or 5-7 days. In some embodiments, the resulting solution can be left under high vacuum, such as around 1 mmHg for about 1 hour.

A certain amount of benzyl alcohol can be removed by high vacuum. The loss of benzyl alcohol can be monitored over time until analysis of the solution indicates that the desired ratio of benzyl alcohol to budiodarone is obtained.

The present disclosure also provides a process for preparing a solution comprising benzyl alcohol and budiodarone in a molar ratio of benzyl alcohol to budiodarone of between about 0.5 to 1 and 4.5 to 1, wherein the budiodarone has an aqueous solubility of about 200 mg/ml to about 1250 mg/ml when contacted with an aqueous solution, and wherein the process comprises:

(a) mixing benzyl alcohol, budiodarone, and water, wherein the benzyl alcohol and budiodarone are present in a molar ratio that is greater than between about 0.5 to 1 and 4.5 to 1; and
(b) removing the water by lyophilization until the formulation comprising benzyl alcohol and budiodarone in a molar ratio of benzyl alcohol to budiodarone of between about 0.5 to 1 and 4.5 to 1 is formed.

In step (a) of the process, the appropriate amounts of benzyl alcohol and budiodarone are mixed with water. In some embodiments, the benzyl alcohol and budiodarone are present in a molar ratio that is greater than between about 0.5 to 1 and 4.5 to 1, such as up to about 11 to 1. In some embodiments, for step (a), the benzyl alcohol and budiodarone are present in a molar ratio of 1 to 1; 1.5 to 1; 2 to 1; 2.5 to 1; 3 to 1; 3.5 to 1; 4 to 1; 4.5 to 1; 5 to 1; 5.5 to 1; 6 to 1; 6.5 to 1; 7 to 1; 7.5 to 1; 8 to 1; 8.5 to 1; 9 to 1; 9.5 to 1; 10 to 1; 10.5 to 1; or 11 to 1. In some embodiments, for step (a), the benzyl alcohol and budiodarone are present in a molar ratio of between about 0.5 to 1 and 11 to 1.

The mixing of benzyl alcohol and budiodarone can be performed at room temperature or with light heating. Light heating can be heating up to 30, 35, or 40° C.

In step (b) of the process, the water is removed by lyophilization. In some embodiments, the solution from step (a) can be frozen below the sublimation temperature of water, and is then lyophilized until analysis shows that the solution has the desired ratio of benzyl alcohol. The preparation method using lyophilization is advantageous in that potentially toxic co-solvents can be avoided in the preparation process. In some embodiments, the lyophilization is performed at room temperature, such as about 18-20, 20-22, 22-24, or 24-26° C. Room temperature includes 18, 19, 20, 21, 22, 23, 24, 25, and 26° C.

The present disclosure provides a method of preparing aqueous solution comprising benzyl alcohol and budiodarone, wherein the budiodarone is present in the solution in a concentration of about 200 mg/ml to about 1250 mg/ml, and wherein the process comprises

(a) the steps from the process of preparing a solution comprising benzyl alcohol and budiodarone in a molar ratio of benzyl alcohol to budiodarone of between about 0.5 to 1 and 4.5 to 1, wherein the budiodarone has an aqueous solubility of about 200 mg/ml to about 1250 mg/ml when contacted with an aqueous solution; and
(b) adding water to the solution of step (a).

In some embodiments, after step (b), the aqueous solution can be agitated. A method of agitation is sonication.

The present disclosure provides a method of preparing aqueous solution comprising benzyl alcohol and budiodarone, wherein the budiodarone is present in the solution in a concentration of about 200 mg/ml to about 1250 mg/ml, and wherein the process comprises

(a) mixing benzyl alcohol and budiodarone; and
(b) adding water to the solution of step (a).

In step (a) of the process, the appropriate amounts of benzyl alcohol and budiodarone are mixed with a co-solvent. In some embodiments, the benzyl alcohol and budiodarone are present in a molar ratio that is greater than between about 0.5 to 1 and 4.5 to 1, such as up to about 11 to 1. In some embodiments, for step (a), the benzyl alcohol and budiodarone are present in a molar ratio of 1 to 1; 1.5 to 1; 2 to 1; 2.5 to 1; 3 to 1; 3.5 to 1; 4 to 1; 4.5 to 1; 5 to 1; 5.5 to 1; 6 to 1; 6.5 to 1; 7 to 1; 7.5 to 1; 8 to 1; 8.5 to 1; 9 to 1; 9.5 to 1; 10 to 1; 10.5 to 1; or 11 to 1.

In some embodiments, after step (b), the aqueous solution can be agitated. A method of agitation is sonication.

The present disclosure provides, in some embodiments, a solution having the characteristics of a solution prepared by the processes disclosed herein.

The present disclosure provides, in some embodiments, a solution having the characteristics of a solution prepared by mixing benzyl alcohol and budiodarone in a sufficient amount to make a clear solution, wherein the benzyl alcohol and budiodarone are present in a molar ratio that is between about 0.5 to 1 and 4.5 to 1.

The present disclosure provides, in some embodiments, a solution having the characteristics of a solution prepared by

(a) mixing benzyl alcohol, budiodarone, and a co-solvent in a sufficient amount to make a clear solution, wherein the benzyl alcohol and budiodarone are present in a molar ratio that is greater than between about 0.5 to 1 and 4.5 to 1; and
(b) removing the co-solvent until the solution comprises benzyl alcohol and budiodarone in a molar ratio of benzyl alcohol to budiodarone of between about 0.5 to 1 and 4.5 to 1.

The present disclosure provides, in some embodiments, a solution having the characteristics of a solution prepared by

(a) mixing benzyl alcohol, budiodarone, and water, wherein the benzyl alcohol and budiodarone are present in a molar ratio that is greater than between about 0.5 to 1 and 4.5 to 1; and
(b) removing the water by lyophilization until the formulation comprising benzyl alcohol and budiodarone in a molar ratio of benzyl alcohol to budiodarone of between about 0.5 to 1 and 4.5 to 1 is formed.

By having the characteristics of a solution prepared by the certain processes, the resulting solution has properties that are same or similar to a solution prepared by the process. In some embodiments, the budiodarone solubility, budiodarone concentration, chemical stability, physical stability, or solution clarity are measured and are the same or similar to a solution prepared by the process.

Analysis of Solution of Budiodarone and Benzyl Alcohol

Mixtures of benzyl alcohol and budiodarone can be analyzed using several different techniques in order to determine the molar ratio of benzyl alcohol to budiodarone. Examples of these are given in the experimental section. Analytical techniques to measure the molar ratio include high-performance liquid chromatography (HPLC), nuclear magnetic resonance spectroscopy (NMR), and mass spectrometry (MS). In some embodiments, a combination of two or more of these techniques is used in order to identify the molecular structure of the constituents of the mixture, in addition to measuring their molar ratio. Examples include HPLC/MS, or HPLC/MS/MS, or HPLC and NMR, NMR and MS, and HPLC/MS/MS and NMR.

¹H-NMR spectrum of benzyl alcohol in CDCl₃ shows 2 benzylic protons at 4.68 ppm and 5 aromatic protons between 7.2 and 7.4 ppm. In the NMR spectrum of the benzyl alcohol/budiodarone mixture, the 2 benzylic protons at 4.68 ppm are isolated from signals of the budiodarone protons and therefore the benzylic signal can be used in order to estimate the molar ratio between the two molecules. The proton NMR spectrum of budiodarone has several well-isolated signals that can be used to calculate the molar ratio. There is a signal at 0.87 ppm (3H, 2-butyl ester group), 1.22 ppm (3H, 2-butyl ester group), 1.41 ppm (6H, N-diethyl group), 4.01 ppm (2H, acetyl protons), and 8.21 ppm (2H, aromatic protons on the iodinated ring). Peak integration gives an accurate measurement of the number of protons associated with a given signal, therefore peak integration can be used to estimate molecular ratios. The approximation of molar ratio can be obtained when dividing peak intensity at 4.68 ppm by peak intensity at 8.21 ppm. Peaks in the upfield area (butyl ester and N-diethyl peaks) also give estimates, but they can be polluted by small amounts of residual alkanes that give signals in the same range. The peak at 4.01 ppm is outside the alkane range, but at higher ratios (such as ratios >5), it appears that the molar ratio measurement using this NMR technique is underestimated.

Methods for Treatment

The present disclosure provides, in some embodiments, a solution comprising benzyl alcohol and budiodarone for treating indications disclosed in U.S. Pat. Nos. 7,498,449, 5,364,880, 5849788, 6130240, 5440054, 6316487, 6372783, 6362223, 6683195, and 6818782, which are hereby incorporated by reference in their entireties. The present disclosure provides, in some embodiments, a solution comprising benzyl alcohol and budiodarone for treating cardiac arrhythmia in a patient. The present disclosure provides, in some embodiments, a solution comprising benzyl alcohol and budiodarone for reducing the incidence of cardiac arrhythmias in a patient.

The present disclosure provides, in some embodiments, a solution comprising benzyl alcohol and budiodarone for restoring sinus and ventricular rhythms in a patient having persistent atrial fibrillation, paroxysmal atrial fibrillation, atrial flutter, ventricular arrhythmias, or ventricular fibrillation. The present disclosure provides, in some embodiments, a solution comprising benzyl alcohol and budiodarone for restoring normal cardiac rhythms in a patient. The present disclosure provides, in some embodiments, a solution comprising benzyl alcohol and budiodarone for reducing atrial fibrillation burden in a patient.

The present disclosure provides, in some embodiments, a solution comprising benzyl alcohol and budiodarone for reducing atrial fibrillation episode duration in a patient. The present disclosure provides, in some embodiments, a solution comprising benzyl alcohol and budiodarone for preventing atrial remodeling in a patient. The present disclosure provides, in some embodiments, a solution comprising benzyl alcohol and budiodarone for reversing atrial remodeling in a patient. The present disclosure provides, in some embodiments, a solution comprising benzyl alcohol and budiodarone for reducing stroke rate in a patient. The present disclosure provides, in some embodiments, a solution comprising benzyl alcohol and budiodarone for increasing time in normal sinus rhythm in a patient. The present disclosure provides, in some embodiments, a solution comprising benzyl alcohol and budiodarone for treating a refractory atrial fibrillation patient, wherein the patient is refractory to one or more anti-arrhythmic drugs, by administering an amount of budiodarone effective to reduce atrial fibrillation burden (AFB).

In the above treatment methods, in some embodiments, the patient being treated is a mammal. Such mammal can be a human or an animal such as a primate (e.g., a monkey, chimpanzee, etc.), a domesticated animal (e.g., a dog, cat, horse, etc.), farm animal (e.g., goat, sheep, pig, cattle, etc.), or laboratory animal (e.g., mouse, rat, etc.). In some embodiments, a patient being treated is a human. In some embodiments, a patient can be a male or female human.

A patient can be a human of any age, such as newborn, young age, adolescent age, adult age, middle age, or old age. In some embodiments, the patient is a newborn. In some embodiments, the patient is of old age. In some embodiments, the patient is of middle age. In some embodiments, the patient can be an age of 0-3, 4-6, 7-9, or 10-12 months. In some embodiments, the patient can be an age of 1-4, 5-9, 10-19, 20-29, 30-39, 40-49, 50-59, 60-69, 70-79, 80-89, or 90-100 years.

In the above treatment methods, in some embodiments, the formulation is administered intravenously. In the above treatment methods, in some embodiments, the formulation is administered intramuscularly. In the above treatment methods, in some embodiments, the formulation is administered subcutaneously. In the above treatment methods, in some embodiments, the formulation is administered orally.

Kits

The present disclosure provides a kit comprising a solution comprising benzyl alcohol and budiodarone. The kit includes one or more containers. A container comprises any of the compositions herein. For example, a container can comprise a solution comprising benzyl alcohol and budiodarone. In certain embodiments, an ampoule can comprise a solution comprising benzyl alcohol and budiodarone. In certain embodiments, a syringe can comprise a solution comprising benzyl alcohol and budiodarone.

The solution of container is administered according to the instructions for use. In certain embodiments, instructions for use are for the treatment of an animal such as a human.

Instruction for use may include information regarding proper diluents and volumes for dilution, if any, of the container. The instructions for use may also provide information regarding the proper administration of the compositions herein, such as frequency and dosage of administration.

The kit may further comprise a syringe or other suitable delivery device for delivering the compositions in the container to a subject. In some embodiments, a syringe or delivery device may be preloaded with a unit dose of a solution of the present invention. In some embodiments, the syringe is a two-barrel syringe in which one barrel contains a solution comprising benzyl alcohol and budiodarone and another barrel contains an aqueous vehicle.

### EMBODIMENTS

### Embodiment 1

A solution comprising benzyl alcohol and budiodarone, wherein the budiodarone has an aqueous solubility of about 200 mg/ml to about 1250 mg/ml when contacted with an aqueous solution.

### Embodiment 2

The solution of Embodiment 1, wherein the aqueous solubility of budiodarone in the solution is 200 mg/ml to 750 mg/ml.

### Embodiment 3

The solution of Embodiment 1, wherein the aqueous solubility of budiodarone in the solution is 750 mg/ml to 1250 mg/ml.

### Embodiment 4

The solution of Embodiment 1, wherein the benzyl alcohol and budiodarone are in a molar ratio of benzyl alcohol to budiodarone of between about 0.5 to 1 and 4.5 to 1.

### Embodiment 5

The solution of Embodiment 4, wherein the solution has characteristics of a solution prepared by:

(a) mixing benzyl alcohol, budiodarone, and a co-solvent in a sufficient amount to make a clear solution, wherein the benzyl alcohol and budiodarone are present in a molar ratio that is greater than between about 0.5 to 1 and 4.5 to 1; and
(b) removing the co-solvent until the solution comprises benzyl alcohol and budiodarone in a molar ratio of benzyl alcohol to budiodarone of between about 0.5 to 1 and 4.5 to 1.

### Embodiment 6

The solution of Embodiment 4, wherein the solution has characteristics of a solution prepared by:

(a) mixing benzyl alcohol, budiodarone, and water, wherein the benzyl alcohol and budiodarone are present in a molar ratio that is greater than between about 0.5 to 1 and 4.5 to 1; and
(b) removing the water by lyophilization until the formulation comprising benzyl alcohol and budiodarone in a molar ratio of benzyl alcohol to budiodarone of between about 0.5 to 1 and 4.5 to 1 is formed.

### Embodiment 7

The solution of any of Embodiments 5-6, wherein the benzyl alcohol and budiodarone are present when mixed in a molar ratio that is between about 0.5 to 1 and 11 to 1 in step (a).

### Embodiment 8

The solution of Embodiment 1, wherein the benzyl alcohol and budiodarone are present in a molar ratio of benzyl alcohol to budiodarone of between about 2 to 1 and 4 to 1.

### Embodiment 9

The solution of Embodiment 1, wherein the benzyl alcohol and budiodarone are present in a molar ratio of benzyl alcohol to budiodarone of about 3 to 1.

### Embodiment 10

The solution of Embodiment 1, wherein the benzyl alcohol and budiodarone are present in a molar ratio of benzyl alcohol to budiodarone of between about 5 to 1 and 11 to 1.

### Embodiment 11

The solution of Embodiment 1, wherein the solution is substantially free of a solubilizing excipient.

### Embodiment 12

The solution of Embodiment 11, wherein the solubilizing excipient is selected from water-soluble organic solvents (polyethylene glycol 300, polyethylene glycol 400, ethanol, propylene glycol, glycerin, N-methyl-2-pyrrolidone, dimethylacetamide, dimethylsulfoxide, etc.), non-ionic surfactants (CREMOPHOR® EL, CREMOPHOR® RH 60, polysorbate 80, SOLUTOL® HS 15, etc.), water-insoluble lipids, phospholipids (usually for injectable emulsions), and various cyclodextrins.

### Embodiment 13

The solution of Embodiment 11, wherein the solubilizing excipient is TWEEN.

### Embodiment 14

The solution of Embodiment 1, wherein the solution comprises an emulsifier selected from labrasol, gelucire, phospholipid, cholesterol, or plant sterols.

### Embodiment 15

The solution of Embodiment 1, wherein the solution is clear.

### Embodiment 16

The solution of Embodiment 1, wherein the solution can be stored for at least 2 years at room temperature or up to around 40° C.

### Embodiment 17

The solution of Embodiment 1, wherein the solution is stored at room temperature or cold temperature.

### Embodiment 18

An aqueous solution comprising benzyl alcohol and budiodarone, wherein the budiodarone is present in the solution in a concentration of about 200 mg/ml to about 1250 mg/ml.

### Embodiment 19

The solution of Embodiment 18, wherein the concentration of budiodarone in the solution is between about 500 and 1250 mg/ml.

### Embodiment 20

The solution of Embodiment 18, wherein the concentration of budiodarone in the solution is between about 800 and 1250 mg/ml.

### Embodiment 21

An aqueous solution comprising benzyl alcohol and budiodarone, wherein the budiodarone is present in the solution at a concentration of between about 0.1 and 500 mg/ml.

### Embodiment 22

The solution of Embodiment 18, wherein benzyl alcohol is present in a concentration of 0.005% to 12.5% v/v.

### Embodiment 23

The solution of Embodiment 18, wherein benzyl alcohol is present in a concentration of 1.5% to 48.5% v/v.

### Embodiment 24

The solution of Embodiment 18, wherein benzyl alcohol is present in a concentration of 4% to 48.5% v/v.

### Embodiment 25

The solution of Embodiment 18, wherein the solution comprises dextrose or sucrose.

### Embodiment 26

The solution of Embodiment 25, wherein dextrose solution is 5% w/v.

### Embodiment 27

The solution of Embodiment 18, wherein the solution comprises an osmolality modifier selected from dimethylacetamide, propylene glycol, and polyethylene glycol.

### Embodiment 28

A capsule comprising the solution of any of Embodiments 1-27.

### Embodiment 29

The capsule of Embodiment 28, wherein the capsule is a gelcap.

### Embodiment 30

The capsule of Embodiment 28, where the capsule further comprises pharmaceutically acceptable excipients.

### Embodiment 31

The capsule of Embodiment 28, wherein the amount of budiodarone is from about 50 mg to about 800 mg.

### Embodiment 32

An oral syrup comprising the solution of any of Embodiments 1-27 and pharmaceutically acceptable excipients.

### Embodiment 33

A kit comprising the solution of any of Embodiments 1-27 and instructions for use.

### Embodiment 34

The kit of Embodiment 33, wherein the formulation is contained in an ampoule or syringe.

### Embodiment 35

A process for preparing a solution comprising benzyl alcohol and budiodarone in a molar ratio of benzyl alcohol to budiodarone of between about 0.5 to 1 and 4.5 to 1, wherein the budiodarone has an aqueous solubility of about 200 mg/ml to about 1250 mg/ml when contacted with an aqueous environment, and wherein the process comprises:

(a) mixing benzyl alcohol, budiodarone, and a co-solvent in a sufficient amount to make a clear solution, wherein the benzyl alcohol and budiodarone are present in a molar ratio that is greater than between about 0.5 to 1 and 4.5 to 1; and
(b) removing the co-solvent until the solution comprises benzyl alcohol and budiodarone in a molar ratio of benzyl alcohol to budiodarone of between about 0.5 to 1 and 4.5 to 1.

### Embodiment 36

The process of Embodiment 35, wherein the benzyl alcohol and budiodarone in step (a) are present in a molar ratio of benzyl alcohol to budiodarone is between about 5 to 1 and 11 to 1 when the benzyl alcohol, budiodarone, and co-solvent are mixed.

### Embodiment 37

The process of Embodiment 35, wherein the co-solvent is selected from methylene chloride, methanol, acetone, ethyl acetate, acetonitrile, and supercritical carbon dioxide.

### Embodiment 38

The process of Embodiment 35, wherein the co-solvent is removed under reduced pressure.

### Embodiment 39

The process of Embodiment 35, wherein the co-solvent is removed under reduced pressure and the resulting solution is left under reduced pressure until a molar ratio of between about 0.5 to 1 and 4.5 to 1 is obtained.

### Embodiment 40

The process of Embodiment 35, further comprising adding water to the solution comprising benzyl alcohol and budiodarone in a molar ratio of benzyl alcohol to budiodarone of between about 0.5 to 1 and 4.5 to 1 after step (b).

### Embodiment 41

A process for preparing a solution comprising benzyl alcohol and budiodarone in a molar ratio of benzyl alcohol to budiodarone of between about 0.5 to 1 and 4.5 to 1, wherein the budiodarone has an aqueous solubility of about 200 mg/ml to about 1250 mg/ml when contacted with an aqueous environment, and wherein the process comprises:

(a) mixing benzyl alcohol, budiodarone, and water, wherein the benzyl alcohol and budiodarone are present in a molar ratio that is greater than between about 0.5 to 1 and 4.5 to 1; and
(b) removing the water by lyophilization until the formulation comprising benzyl alcohol and budiodarone in a molar ratio of benzyl alcohol to budiodarone of between about 0.5 to 1 and 4.5 to 1 is formed.

### Embodiment 42

The process of Embodiment 41, wherein the benzyl alcohol and budiodarone in step (a) are present in a molar ratio of benzyl alcohol to budiodarone that is between about 5 to 1 and 11 to 1 when the benzyl alcohol, budiodarone, and water are mixed.

### Embodiment 43

A method of treating cardiac arrhythmia or reducing the incidence of cardiac arrhythmias comprising administering to a patient in need thereof the solution of any of claims 1-27.

### Embodiment 44

A method of restoring sinus and ventricular rhythms in a patient having persistent atrial fibrillation, paroxysmal atrial fibrillation, atrial flutter, ventricular arrhythmias, or ventricular fibrillation comprising administering to a patient in need thereof the solution of any of claims 1-27.

### Embodiment 45

A method of restoring normal cardiac rhythms in a patient comprising administering to the patient in need thereof the solution of any of Embodiments 1-27.

### Embodiment 46

A method of reducing atrial fibrillation burden in a patient comprising administering to the patient in need thereof the solution of any of Embodiments 1-27.

### Embodiment 47

A method for reducing atrial fibrillation episode duration in a patient comprising administering to the patient in need thereof the solution of any of Embodiments 1-27.

### Embodiment 48

A method for preventing atrial remodeling in a patient comprising administering to the patient in need thereof the solution of any of Embodiments 1-27.

### Embodiment 49

A method for reversing atrial remodeling in a patient comprising administering to the patient in need thereof the solution of any of Embodiments 1-27.

### Embodiment 50

A method for reducing stroke rate in a patient comprising administering to the patient in need thereof the solution of any of Embodiments 1-27.

### Embodiment 51

A method for increasing time in normal sinus rhythm in a patient comprising administering to the patient in need thereof the solution of any of Embodiments 1-27.

### Embodiment 52

A method for treating a refractory atrial fibrillation patient comprising administering to patient refractory to one or more anti-arrhythmic drugs the solution of any of Embodiments 1-27 in an effective amount to reduce atrial fibrillation burden (AFB).

### Embodiment 53

The method of any of Embodiments 43-52, wherein the formulation is administered intravenously.

### Embodiment 54

The method of any of Embodiments 43-52, wherein the formulation is administered orally.

### Embodiment 55

The method of any of Embodiments 43-52, wherein the patient is a newborn.

### Embodiment 56

The method of any of Embodiments 43-52, wherein the patient is of middle age or old age.

### EXAMPLES

The following examples are offered to illustrate but not to limit the invention.

### Example 1

Analyses of Samples

Vials containing the samples to be analyzed were vortexed on a Genie 2 vortexer in order to homogenized potential heterogeneous contents and an aliquot (500 to 1000 μl) was taken into a 1-ml microfuge tube. Samples were then centrifuged at 13,000 rpm for 10 minutes using an X Systems Abbott Medical lab centrifuge Model 3531 equipped with a Heraeus Sepatech 3734 rotor.

Following centrifugation, an aliquot (40 to 100 μl) was diluted with methanol so that the final budiodarone concentration is between 0.1 and 1 mg/ml. These dilutions were then analyzed by HPLC.

### HPLC Conditions 1:

UV detector set at 220 nm

### Column: Symmetry C18 3.5 μm (4.6×75 mm)

Mobile phase: (A) 0.1% trifluoroacetic acid in acetonitrile (B) 0.1% trifluoroacetic acid in water.
(A)/(B) from 5/95 to 100/0 in 9 minutes at 1.0 ml/min
Retention times=4.33 minutes (benzyl alcohol) and 6.47 minutes (budiodarone)

### HPLC Conditions 2:

UV detector set at 220 nm

### Column: ZorbaxC8 3.5 μm (4.6×75 mm)

Mobile phase: (A) 0.1% trifluoroacetic acid in acetonitrile (B) 0.1% trifluoroacetic acid in water.
(A)/(B) from 30/70 to 100/0 in 7 minutes at 2.0 ml/min
Retention times=1.87 minutes (benzyl alcohol) and 6.39 minutes (budiodarone)

### HPLC Conditions 3:

UV detector set at 220 nm

### Column: ZorbaxC8 3.5 μm (4.6×75 mm)

Mobile phase: (A) 0.1% trifluoroacetic acid in acetonitrile (B) 0.1% trifluoroacetic acid in water.
(A)/(B) from 30/70 to 100/0 in 4 minutes then 100/0 for 1 minute at 2.0 ml/min
Retention times=1.84 minutes (benzyl alcohol) and 5.13 minutes (budiodarone)

### Example 2

### Solubility of Budiodarone Tartrate in Water

The aqueous solubility of budiodarone tartrate was measured in pure distilled water. Budiodarone tartrate (250 mg) was suspended in distilled water (10 ml) and the suspension was vortexed on a VWR Genie 2 vortexer at maximum speed for 3×10 sec duration. The suspension was then stirred for 96 hours at room temperature, protected from light. After 96 hours, the suspension was vortexed again and a 1 ml aliquot was centrifuged at 13,000 rpm for 10 minutes to precipitate all insoluble material. A 40 μl aliquot of the clear supernatant was diluted with 960 μl of methanol (25-fold dilution) and was analyzed by HPLC using Conditions 3. No significant amount of degradation products were detected in the clear supernatant.

The amount of budiodarone in solution is found to be 7.04 mg/ml. This figure is the solubility of budiodarone tartrate in water.

### Example 3

### Solubility of Budiodarone Tartrate in Water Containing Various Amounts of Benzyl Alcohol

Three series of samples were prepared. To budiodarone tartrate (50 mg, 100 mg, and 200 mg) in 4-ml screw-cap glass vials was added water, followed by benzyl alcohol in the proportions showed in Tables 1 to 3. The vials were then capped and the mixtures were vortexed on a VWR Genie 2 vortexer at maximum speed for 3×10 sec duration. The final concentrations of budiodarone were 25 mg/ml, 50 mg/ml, and 100 mg/ml in water containing benzyl alcohol from 0.5% to 11% v/v. The vials were kept in the dark for 24 hours and analyzed by HPLC as per Example 1 Conditions 1.

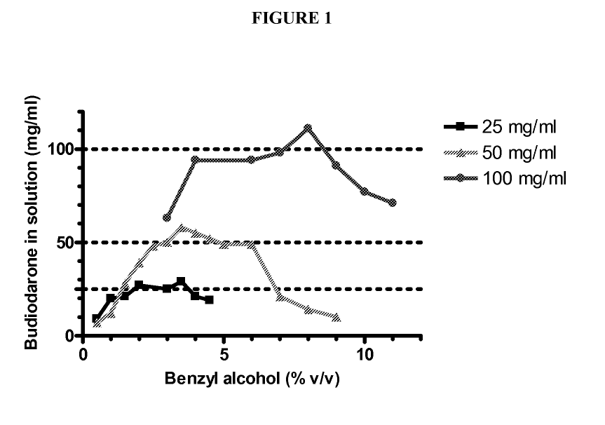

The results are shown in FIG. 1, where the amount of budiodarone in solution is plotted as a function of the benzyl alcohol concentration. FIG. 1 shows that there is a lower limit and a higher limit to the benzyl alcohol concentration within which budiodarone tartrate is fully soluble in water. For example, at 25 mg/ml, budiodarone is soluble in approximately 1.5% to 4.5% v/v of benzyl alcohol. At 50 mg/ml, budiodarone is soluble in approximately 3% to 6% benzyl alcohol. At 100 mg/ml, budiodarone is soluble in approximately 4% to 8% benzyl alcohol.

TABLE 1
Preparation of 25 mg/ml budiodarone tartrate in 0.5% to 4.5% benzyl alcohol

| Vial # | 1 | 2 | 3 | 4 | 5 | 6 | 7 | 8 | 9 |
|---|---|---|---|---|---|---|---|---|---|
| Budiodarone | 50 | 50 | 50 | 50 | 50 | 50 | 50 | 50 | 50 |
| tartrate (mg) | | | | | | | | | |
| Benzyl | 10 | 20 | 30 | 40 | 50 | 60 | 70 | 80 | 90 |
| alcohol (μl) | | | | | | | | | |
| Water (μl) | 1990 | 1980 | 1970 | 1960 | 1950 | 1940 | 1930 | 1920 | 1910 |

TABLE 2
Preparation of 50 mg/ml budiodarone
tartrate in 0.5% to 4.5% benzyl alcohol

| Vial # | 1 | 2 | 3 | 4 | 5 | 6 | 7 | 8 |
|---|---|---|---|---|---|---|---|---|
| Budiodarone | 100 | 100 | 100 | 100 | 100 | 100 | 100 | 100 |
| tartrate (mg) | | | | | | | | |
| Benzyl | 20 | 40 | 60 | 80 | 100 | 120 | 140 | 160 |
| alcohol (μl) | | | | | | | | |
| Water (μl) | 1980 | 1960 | 1940 | 1920 | 1900 | 1880 | 1860 | 1840 |

TABLE 3
Preparation of 100 mg/ml budiodarone tartrate in 0.5% to 4.5% benzyl alcohol

| Vial # | 1 | 2 | 3 | 4 | 5 | 6 | 7 | 8 | 9 |
|---|---|---|---|---|---|---|---|---|---|
| Budiodarone | 200 | 200 | 200 | 200 | 200 | 200 | 200 | 200 | 200 |
| tartrate (mg) | | | | | | | | | |
| Benzyl | 60 | 80 | 100 | 120 | 140 | 160 | 180 | 200 | 220 |
| alcohol (μl) | | | | | | | | | |
| Water (μl) | 1940 | 1920 | 1900 | 1880 | 1860 | 1840 | 1920 | 1800 | 1780 |

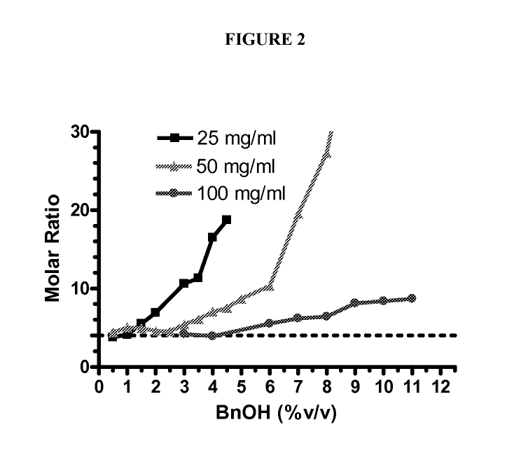

In FIG. 2, the molar ratio of benzyl alcohol to budiodarone in solution was plotted against the concentration of benzyl alcohol. FIG. 2 shows that there is a lower limit to the molar ratio that forms in solution. This lower-limit is approximately 5/1 of benzyl alcohol to budiodarone and forms even when the molar amount of benzyl alcohol added to the sample is less than 5-fold the molar amount of budiodarone. In other words, FIG. 2 shows that budiodarone is not soluble in water unless there is a 5-fold molar excess of benzyl alcohol. For example, if 3% benzyl alcohol were added to a 100 mg/ml suspension of budiodarone in water, which represents only a 3.2 molar excess, then an approximately 5/1 molar ratio between benzyl alcohol and budiodarone forms in solution, and the excess budiodarone tartrate precipitates out. When the molar excess of benzyl alcohol reaches 5/1 or greater, then the entire amount of budiodarone is soluble and there is a linear relationship between the amount of benzyl alcohol and the molar ratio of benzyl alcohol to budiodarone.

Above an approximately 11/1 molar ratio, budiodarone was not fully soluble in water anymore. A phase separation was observed. Benzyl alcohol separated from the aqueous phase and sank to the bottom of the vial. As budiodarone is more soluble in benzyl alcohol than in water, budiodarone becomes solubilized in the benzyl alcohol layer and the concentration of budiodarone in the aqueous layer decreases (FIG. 1) and the relationship is no longer linear (FIG. 2).

### Example 4

### Solubility of Budiodarone Tartrate and Benzyl Alcohol in Solvents of Different Ionic Strengths

Data from Example 3 suggest that a molecular association of some sort exists between budiodarone and benzyl alcohol, i.e., a colloid made of nanometer-sized particles dispersed in water. Van der Waals attractive forces and ionic repulsive forces balance each other and Brownian motion keeps the nanometer-sized particle from sedimenting to the bottom of the flask. If this is indeed true, then changing the ionic strength of the solvent should disturb this balance and budiodarone should precipitate out of solution.

To find out, five vials, each one containing budiodarone tartrate (200 mg, 0.234 mmoles) and benzyl alcohol (122 μl, 1.173 mmoles) were prepared. The molar ratio was therefore 5/1. To each of these vials was added 4 ml of water and the vials were vortexed, resulting in clear solutions containing 50 mg/ml of budiodarone tartrate and benzyl alcohol (molar ratio of 5/1). Then, 4 ml of one of the following solvents was added: 30 mM acetate buffer pH 5.2, 30 mM phosphate buffer saline pH 7.4, 30 mM Tris.HCl buffer pH 7.5, 1.8% sodium chloride (saline solution), and 10% dextrose solution (D10W) resulting in 15 mM concentration of buffer, 0.9% concentration of sodium chloride, and 5% concentration of dextrose (D5W), respectively.

Upon addition of an ionic solvent, all the samples except the D5W sample became turbid. The vials were then vortexed at high speed (3×10 sec) and left to stand for 1 hour. Then they were vortexed again (3×10 sec) and a 1-ml aliquot from each vial was centrifuged at 13,000 rpm for 10 min. A small aliquot of the clear supernatant was diluted 25-fold with methanol and analyzed by HPLC (Conditions 3). The remaining concentrations of budiodarone in the samples were as follows:

In the Acetate buffer pH5.2: <1 mg/ml
In the Phosphate buffer saline pH 7.4: <1 mg/ml
In the Tris.HCl buffer pH7.5: <1 mg/ml
In the 0.9% saline solution: <1 mg/ml
In the D5W: 26.6 mg/ml

The results show that there was almost no budiodarone left in solution in the vials containing solvents of high ionic strength (the 3 buffers and the saline solution), whereas budiodarone is still in solution (25 mg/ml) in the vial containing D5W, a solvent of low ionic strength. These data support the formation of a colloid in water between budiodarone and benzyl alcohol, where modifying the ionic strength of the solvent results in a disturbance of the balance between attractive van der Waals forces and repulsive ionic forces.

### Example 5

### Solubility of Budiodarone Tartrate and Benzyl Alcohol in Different Co-Solvents

Co-solvents that are routinely used in injectable pharmaceutical preparations were studied. These were dimethylacetamide (DMA), dimethylsulfoxide (DMSO), ethanol (EtOH), and N-methylpyrrolidone (NMP). Samples were prepared as described in Table 4 below.

TABLE 4
Preparations containing 25 mg/ml of budiodarone tartrate in various
concentrations of several co-solvents.

| Budiodarone | DMA | | | | |
|---|---|---|---|---|---|
| tartrate (mg) | (μl) | DMSO (μl) | EtOH (μl) | NMP (μl) | Water (μl) |
| 25 | 36 | | | | 964 |
| 25 | 72 | | | | 928 |
| 25 | 144 | | | | 856 |
| 25 | | 36 | | | 964 |
| 25 | | 72 | | | 928 |
| 25 | | 144 | | | 856 |
| 25 | | | 36 | | 964 |
| 25 | | | 72 | | 928 |
| 25 | | | 144 | | 856 |
| 25 | | | | 36 | 964 |
| 25 | | | | 72 | 928 |
| 25 | | | | 144 | 856 |

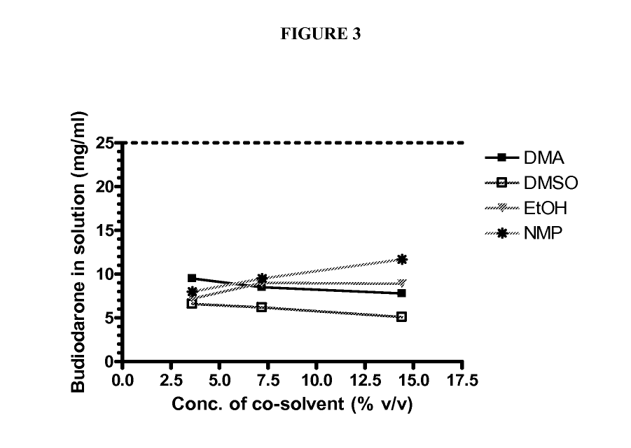

Samples were prepared by mixing all three components together as in previous examples, using a vortexer at maximum speed for 3×10 sec. Samples were then left in the dark for 24 hours and analyzed by HPLC following centrifugation to precipitate any insoluble material. The results are shown in FIG. 3.

If the amounts of soluble budiodarone in these four preparations are compared to the results of Example 2 (solubility in pure water), FIG. 3 shows that in the presence of DMA, DMSO, EtOH, or NMP, the amount of solubilized budiodarone is not significantly different from the amount solubilized in water alone. Of the four co-solvents tested, only NMP seems to have a minimal effect on budiodarone solubility. These four common co-solvents, at concentrations between 3.6% and 14.4% (roughly similar to the concentration range of benzyl alcohol at which budiodarone is fully soluble over a wide range of concentrations from 25 mg/ml to 100 mg/ml) have negligible effect on budiodarone solubility. This is unlike the solvating effect of benzyl alcohol where the entire amount of budiodarone (25 mg) was soluble in 1 ml of water containing as low as 1.5% of benzyl alcohol. In addition, as can be seen in FIG. 1 in the ascending part of the concentration curve, increasing amounts of benzyl alcohol dissolve increasing amounts of budiodarone in such a way that the molar ratio of benzyl alcohol to budiodarone stays at around 5/1. But in FIG. 3, there is no such relationship. Increasing amounts of co-solvents do not dissolve increasing amounts of budiodarone; therefore the molar ratio is not constant. In the case of NMP, there is a small increase in solubility with increasing amounts of NMP, but the molar ratio is not constant. It can therefore be concluded that among co-solvents that are routinely used in injectable preparations, benzyl alcohol seems to have a special set of properties toward budiodarone that cannot be inferred from studying other co-solvents.

### Example 6

### Determination of a Lower-Limit Ratio for Soluble Benzyl Alcohol and Budiodarone Compositions from Mixture of Benzyl Alcohol and Budiodarone in Water

Three series of 20-ml screw-cap glass vials were prepared as described in Tables 5 to 7. Budiodarone tartrate was added to the empty vials, followed by water, and then benzyl alcohol was added. The vials were capped and the mixtures were vortexed for three times 10 seconds. The vials were then left in the dark for 24 hours and were then analyzed as described above using HPLC Conditions 2 and 3.

TABLE 5
Preparations containing 25 mg/ml of budiodarone tartrate

| Budiodarone tartrate (mg) | 250 | 250 | 250 | 250 | 250 | 250 |
|---|---|---|---|---|---|---|
| Benzyl alcohol (μl) | 25 | 50 | 75 | 100 | 125 | 150 |
| Water (μl) | 9,975 | 9,950 | 9,925 | 9,900 | 9,875 | 9,850 |
| Benzyl alcohol (% v/v) | 0.25 | 0.50 | 0.75 | 1.00 | 1.25 | 1.50 |
| Molar ratio | 0.8 | 1.6 | 2.4 | 3.2 | 4.1 | 4.9 |

TABLE 6
Preparations containing 50 mg/ml of budiodarone tartrate

| Budiodarone tartrate (mg) | 250 | 250 | 250 | 250 | 250 | 250 |
|---|---|---|---|---|---|---|
| Benzyl alcohol (μl) | 25 | 50 | 75 | 100 | 125 | 150 |
| Water (μl) | 4,975 | 4,950 | 4,925 | 4,900 | 4,875 | 4,850 |
| Benzyl alcohol (% v/v) | 0.50 | 1.00 | 1.50 | 2.00 | 2.50 | 3.00 |
| Molar ratio | 0.8 | 1.6 | 2.4 | 3.2 | 4.1 | 4.9 |

TABLE 7
Preparations containing 100 mg/ml of budiodarone tartrate

| Budiodarone tartrate (mg) | 250 | 250 | 250 | 250 | 250 | 250 |
|---|---|---|---|---|---|---|
| Benzyl alcohol (μl) | 25 | 50 | 75 | 100 | 125 | 150 |
| Water (μl) | 2,475 | 2,450 | 2,425 | 2,400 | 2,375 | 2,350 |
| Benzyl alcohol (% v/v) | 1.00 | 2.00 | 3.00 | 4.00 | 5.00 | 6.00 |
| Molar ratio | 0.8 | 1.6 | 2.4 | 3.2 | 4.1 | 4.9 |

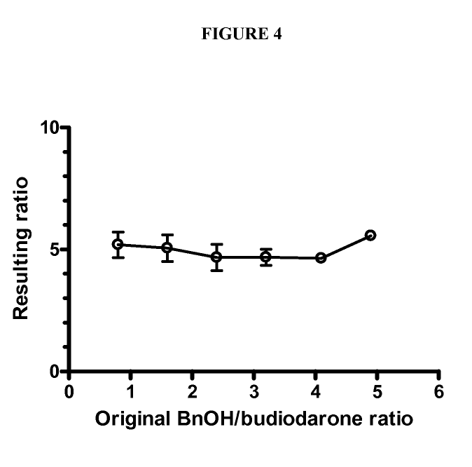

The findings are shown in FIG. 4. In FIG. 4, the approximately 5/1 lower-limit molar ratio is constant over a wide range of budiodarone concentrations, at least between budiodarone concentrations of 25 mg/ml and 100 mg/ml. The resulting ratios in solution, following 24-hour in the dark, were calculated from HPLC analysis data and were plotted versus the actual ratios introduced into the vials. Each point represents the mean findings of the three concentrations (25 mg/ml, 50 mg/ml, and 100 mg/ml)+/−standard deviation. The results show that there is a strong thermodynamic drive for budiodarone to associate with a minimum of approximately 5 molecules of benzyl alcohol in the presence of water molecules. This association takes place even when the original ratio introduced into the vial is as low as 0.8/1. Presumably the 5/1 ratio is an optimum molar ratio allowing for the optimum balance of repulsive ionic forces and attractive van der Waals forces between water molecules and the budiodarone/benzyl alcohol complex. It is also shown that higher ratios are less thermodynamically stable than the 5/1 ratio at room temperature, otherwise these ratios would form preferentially to the 5/1 ratio and the observed ratio would be greater. The 5/1 ratio between benzyl alcohol and budiodarone occurs at concentrations between 25 mg/ml and 100 mg/ml. The 5/1 ratio is therefore the thermodynamically optimum ratio in water at room temperature (22±4° C.). It would then appear that the 5/1 ratio is the ratio of choice. This ratio roughly corresponds to a 3% benzyl alcohol concentration in a 50 mg/ml budiodarone aqueous solution. It is an acceptable level as per World Health Organization (WHO) recommendations, but it would be preferable to minimize the amount of benzyl alcohol to lower the risks of potential toxicity to a subject.

### Example 7

### Preparation of Lower Molecular Ratios (Lower than 5/1) of Benzyl Alcohol to Budiodarone Compositions

A clear solution containing 25 mg/ml of budiodarone and 0.92% (v/v) of benzyl alcohol (molar ratio of 3/1) was prepared. In this example, the association between budiodarone and benzyl alcohol was pre-formed before water is added.

Methanol (5 ml), budiodarone tartrate (250 mg, 0.293 mmole), and benzyl alcohol (92 μl, 0.880 mmole) were mixed. In this mixture, the molar ratio of benzyl alcohol to budiodarone was 3/1. Methanol was removed at 36° C. under partial vacuum (30 mm Hg) to form a clear oily residue. The residue was left at room temperature under high vacuum (1 mmHg) for 1 hour to remove traces of methanol. Water (10 ml), is then added, and the sample is vortexed for 3×10 sec.

In order to confirm the solubility of budiodarone and the molar ratio between benzyl alcohol and budiodarone, the sample was analyzed by HPLC. The analysis was performed immediately after preparation of the solution. An aliquot of the solution was centrifuged as described above in order to remove insoluble material. The supernatant was then analyzed by HPLC and the results indicate that the 3/1 molar ratio was water soluble.

This was unexpected since previous experiments show that the 5/1 ratio forms preferentially in water. This finding shows that low ratios of benzyl alcohol and budiodarone can be prepared and can be diluted in water to make an injectable formulation, provided that benzyl alcohol and budiodarone be mixed together before water is added.

### Example 8

### Preparation and Stability of Lower Molecular Ratios (Lower that 5/1) of Benzyl Alcohol to Budiodarone

Samples 1-3 were prepared and tested as shown in Table 8. Samples 1-3 contained different molecular ratios of benzyl alcohol to budiodarone. The stability of these solutions was tested.

Sample 1:

An empty flask (60.398 g) was weighed. Into the flask, budiodarone tartrate (250 mg, 0.293 mmole), benzyl alcohol (924 0.884 mmole), and methanol (5 ml) were added. This was a 3/1 molar ratio of benzyl alcohol to budiodarone. The methanol was removed at 36° C. under partial vacuum (30 mmHg). The flask with the contents was left under high vacuum (1 mmHg) for 96 hours. The flask was weighed again (60.696 g). The amount of residue was 298 mg. The residual amount of benzyl alcohol was 298−250=48 mg, or 0.444 mmole. The resulting benzyl alcohol/budiodarone molar ratio was therefore 0.444/0.293=1.5. Water (10 ml) was added to the residue and the residue fully dissolved. There was no sign of insoluble material. Therefore, a ratio as low as 1.5 was soluble in water. A sample was centrifuged as described above and was analyzed by HPLC following Conditions 3. HPLC indicated that the budiodarone concentration was 26 mg/ml, which agreed with the original concentration of 25 mg/ml. Therefore, budiodarone was fully soluble in water when associated with 1.5 molecules of benzyl alcohol per molecule of budiodarone (Table 8).

Sample 2:

An empty flask (68.867 g) was weighed. Into the flask, budiodarone (250 mg, 0.293 mmole), benzyl alcohol (300 μl, 2.885 mmole), and water (9,700 μl) were added. This was a 9.8/1 ratio. A homogeneous solution was formed. The solution was frozen by immersion into a dry-ice/acetone bath and was then lyophilized at 1 mmHg for 96 hours. The flask was weighed again (69.216 g). The residue weighed 349 mg. The amount of residual benzyl alcohol was therefore 349−250=99 mg, or 0.917 mmole, representing a 3.1/1 molar ratio with budiodarone. Water (10 ml) was added and the residue dissolved completely. HPLC analysis following centrifugation found a 3.3/1 ratio, therefore in good agreement with the weight of the residue, and a budiodarone concentration of 26.3 mg/ml, which agreed with the previous observation that a preformed ratio of 3/1 was water soluble (Table 8).

Sample 3:

An empty flask (69.305 g) was weighed. Benzyl alcohol (2000 and water (10 ml) were added. The solution was frozen in a dry-ice/acetone bath and lyophilized for 96 hours. The flask was weighed (69.307 g). Therefore there was nothing left in the flask. Water (10 ml) was added and HPLC analysis confirmed that there was no benzyl alcohol left in the flask.

Results:

Samples 1 and 2 were analyzed by HPLC over time (Conditions 3). Results are shown in Table 8.

TABLE 8
Stability of budiodarone tartrate (25 mg/ml) in water containing low-
concentrations of benzyl alcohol.

| | Sample1 | | | Sample 2 | | |
|---|---|---|---|---|---|---|
| | | Conc | | | Conc | Molar |
| Time (hr) | AUC | (mg/ml) | Molar ratio | AUC | (mg/ml) | ratio |
| 0 | 195873 | 26.0 | 1.23 | 197816 | 26.3 | 3.30 |
| 24 | 196822 | 26.1 | 1.28 | 196101 | 26.0 | 3.32 |
| 48 | 202688 | 26.9 | 1.36 | 210889 | 28.2 | 3.04 |
| 168 | 136135 | 17.5 | 1.86 | 200369 | 26.7 | 3.28 |
| 216 | 124996 | 15.8 | 2.06 | 197859 | 26.3 | 3.38 |
| 312 | 89787 | 10.8 | 3.09 | 161730 | 21.1 | 3.77 |

In conclusion, this experiment demonstrates that benzyl alcohol in water can evaporate rapidly when lyophilized, but evaporates slowly when budiodarone is present. This is an indication that budiodarone and benzyl alcohol form some kind of molecular association.

Two compositions of different ratios of benzyl alcohol and budiodarone were prepared. One was prepared in a 3/1 ratio using methanol as a low-boiling co-solvent, and the other was prepared in a 9.8/1 ratio using water as a co-solvent. In both cases, the residual weight of benzyl alcohol after 96 hours in a high vacuum was less than the amount of benzyl alcohol added to the flask. This method therefore is a reliable method to prepare solutions of budiodarone in benzyl alcohol of specific molar ratios. Weighing the residual amount of material following high vacuum allows for a reliable estimate of the residual molar ratio. HPLC analysis is also a reliable way to measure the ratio in an unknown sample. Here, ratios of 1.23 and 3.3 were determined by HPLC when the actual ratios are 1.5 and 3.1.

Therefore, there are two different ways to prepare a solution of benzyl alcohol and budiodarone of a ratio less than 5/1. One way is by dissolving the desired amounts of budiodarone and benzyl alcohol in an organic solvent of low boiling point. This solution is then removed under partial vacuum and is then left under high vacuum until the desired weight is obtained. The other way is to prepare a solution of budiodarone and benzyl alcohol in water in a molar ratio of between 5/1 and 11/1 in order to obtain a clear solution, as per Example 3. This solution is then rapidly frozen below the sublimation temperature of water and is lyophilized until the desired weight is obtained.

In this example, a solution of benzyl alcohol and budiodarone can be prepared in ratios as low as 1.5 (1.23 measured by HPLC, Table 8). These solutions are water soluble, which is surprising considering the results obtained in Example 3 where budiodarone was not fully soluble unless the molar ratio of benzyl alcohol/budiodarone was at least 5/1.

In addition, these low ratios are stable for a significant amount of time. In Sample 1, the low ratio of 1.5 is stable for 24 to 48 hours at room temperature. The molar ratio of approximately 3.1 is stable for an even longer amount of time at room temperature.

### Example 9

### Solution with Low Ratio of Benzyl Alcohol to Budiodarone in 5% Dextrose Solution

To a 20-ml screw-cap glass vial was added budiodarone (125 mg, 0.146 mmoles), benzyl alcohol (46 μl, 0.444 mmole), and methanol (5 ml). A clear solution at a ratio of 3/1 formed. The methanol was evaporated at 36° C. under partial vacuum (30 mmHg) to yield a thin film. To this residue was added 5 ml of water. The sample was then vortexed and a clear 25 mg/ml solution of budiodarone tartrate was obtained. A small aliquot was diluted 25 times with methanol and analyzed by HPLC (Sample 1). To the flask was then added 250 mg of D-(+)-glucose (also known as dextrose), resulting in a clear solution of 25 mg/ml budiodarone tartrate in 5% dextrose (D5W). A small sample was analyzed by HPLC (Sample 2). HPLC analysis revealed that some benzyl alcohol was lost under high vacuum, and the resulting molar ratio was 2.15/1.

### Results:

| | |
|---|---|
| Sample1 (solvent = | Budiodarone AUC = 219591; conc = 29.4 mg/ml |
| water) | Benzyl alcohol AUC = 20021; conc = 0.76% |
| | Molar ratio = 2.14 |
| Sample2 (solvent = | Budiodarone AUC = 217419; conc = 29.1 mg/ml |
| D5W) | Benzyl alcohol AUC = 19992; conc = 0.76% |
| | Molar ratio = 2.15 |

Sample 2 is then analyzed over time to check physical and/or chemical stability in solution (Table 9).

TABLE 9
Stability of budiodarone tartrate (25 mg/ml) in D5W at room temperature
in the presence of a 2.15 molar excess of benzyl alcohol.

| | Time (hr) | | |
|---|---|---|---|
| | 0 | 120 | 168 |
| Budiodarone (AUC) | 217419 | 197717 | 183143 |
| Benzyl alcohol (AUC) | 19992 | 18707 | 16867 |
| Budiodarone (mg/ml) | 29.1 | 26.3 | 24.2 |
| Molar ratio | 2.15 | 2.19 | 2.07 |

There was no loss of material due to precipitation when glucose is added to an aqueous solution of 25 mg/ml of budiodarone in 0.76% benzyl alcohol (molar ratio=2.14). This conclusion can be reached by comparing the analysis results of Sample 1 with Sample 2. Therefore, it is possible to make an injectable formulation that is isotonic to blood using dextrose as an osmotic agent.

Also, the low ratio of 2.14/1 in D5W is stable for many hours. Table 9 shows that the 2.14/1 molar ratio is stable for up to about 120 hours. At 120 hours, the amount of budiodarone in solution has not decreased significantly but the solution started showing some turbidity. At 168 hours, the amount of budiodarone in solution has decreased significantly.

### Example 10

### Comparison of Stability Between Solutions in Water and Solutions in D5W

This example shows the effects of adding a non-ionic solute, such as D5W, on the stability of low-ratio solutions.

TABLE 10
Preparation of 25 mg/ml solutions of budiodarone tartrate in water and in
D5W containing various concentrations of benzyl alcohol.

| | Budiodarone | Benzyl | |
|---|---|---|---|
| Vial # | tartrate (mg) | alcohol (μl) | Methanol (μl) |
| D1 | 250 | 30.5 | 4 |
| D2 | 250 | 61 | 4 |
| D3 | 250 | 91.5 | 4 |
| W1 | 250 | 30.5 | 4 |
| W2 | 250 | 61 | 4 |
| W3 | 250 | 91.5 | 4 |

Six vials as in Table 10 were prepared. The methanol was evaporated at 36° C. and under 30 mmHg vacuum. The vials were then put together in a single container and they were left under high vacuum (<3 mmHg) for 60 minutes. Samples D1 to D3 were then dissolved in 10 ml of D5W, whereas samples W1 to W3 were dissolved in 10 ml of distilled water. Up to this point all samples have been treated under the exact same conditions, therefore the residual amounts of benzyl alcohol in D1, D2, and D3 should be the same as in W1, W2, and W3, respectively. The final concentration of budiodarone in each vial was then 25 mg/ml (HPLC results may differ slightly because of sampling errors and propagation of errors in the analysis protocol). The molar ratio of benzyl alcohol to budiodarone was 1/1 in D1 and in W1, 2/1 in D2 and in W2, and 3/1 in D3 and in W3. Aliquots of each vial are analyzed as in previous examples following centrifugation and using HPLC Conditions 3 (Table 11).

TABLE 11
Budiodarone concentrations (mg/ml) as a function of time (hours)

| Sample # | T₀ | T₂ | T₂₄ | T₄₈ | T₁₄₄ | T₁₉₂ |
|---|---|---|---|---|---|---|
| D1 | 26.4 | 29.1 | 24.9 | 18.6 | 8.0 | 10.5 |
| D2 | 27.3 | 28.4 | 27.9 | 27.4 | 19.8 | 18.7 |
| D3 | 28.4 | 28.7 | 29.4 | 27.7 | 25.0 | 23.6 |
| W1 | 28.4 | 27.1 | 24.0 | 17.7 | 8.9 | 11.5 |
| W2 | 28.8 | 28.8 | 28.9 | 27.1 | 17.9 | 16.2 |
| W3 | 27.6 | 29.6 | 29.9 | 30.4 | 26.9 | 23.4 |

Within 2 hours, samples D1 and W1 showed an increased opalescence but no particulates. Centrifugation followed by HPLC confirmed that these samples were still stable at 2 hours (T₂). However, at 24 hours (T₂₄), a significant amount of precipitated material can be seen at the bottom of the vials. HPLC analysis confirmed the loss of material in D1 and in W1 at 24 hours (Table 11). So the 1/1 ratio was stable for at least 2 hours but less than 24 hours at room temperature. The 2/1 ratio (D2 and W2) was stable for at least 48 hours but less than 144 hours at room temperature, and the 3/1 ratio was stable for 6 to 8 days at room temperature.

### Example 11

### Formulation of a Pre-Formed Solution for Reconstitution Before Administration

For intravenous administrations, the sum of all solutes concentrations per unit volume in the injectable formulation (molality of the solution) should be close to the sum of all solutes concentrations in blood, so as to avoid potential risks of hemolysis, phlebitis, or venous thrombosis. In this example we demonstrate that the solution with a low-ratio of benzyl alcohol and budiodarone can be mixed with a pharmaceutically acceptable water-soluble co-solvent to form a fluid oil that is easy to handle in manufacturing processes. The amount of co-solvent is chosen so that the osmolality of the final formulation upon reconstitution with water will approximate the osmolality of blood.

In this example, formulations of pre-formed solutions with additives, such as an osmolality modifier, were prepared. Polyethylene glycol molecular weight 380 to 420 (PEG400), propylene glycol (PG), and dimethylacetamide (DMA) were used. The concentration of budiodarone tartrate was 25 mg/ml and the concentration of benzyl alcohol was 0.92%, corresponding to a 3/1 molar ratio with budiodarone.

For calculation of molality (in mOsm/g of solution): for undissociated small molecules (PEG400, PG, DMA, and benzyl alcohol), molality was assumed to be equal to their molar concentration. For budiodarone tartrate, the molality was calculated to be twice the molar concentration since there are 2 free molecules per molecule of budiodarone tartrate. Since the solvent is water, of density 1 g/ml, then 1 gram of solution (water plus all the solutes) is equivalent to 1 ml. The unit is mOsm/ml.

The molality of 25 mg/ml of budiodarone tartrate (0.0293 mM) is 0.0293×2=0.059 mOsm/ml.

The molality of 0.92% of benzyl alcohol (0.089 mM) is 0.089 mOsm/ml.

The molality of human blood is approximately 0.310 mOsm/ml. Benzyl alcohol (0.92%) and budiodarone tartrate (25 mg/ml) together have a molality of 0.059+0.088=0.147 mOsm/ml, a co-solvent having an approximate molality of 0.310-0.147=0.163 mOsm/ml therefore is used to prepare a suitable injectable formulation that is isotonic to blood.

The molality of 1% PG is 0.136 mOsm/ml.

The molality of 1% DMA is 0.108 mOsm/ml.

The molality of 1% PEG400 is 0.028 mOsm/ml.

Therefore, in order to prepare an isotonic injectable solution, 120 μl of PG, or 150 μl of DMA, or 582 μl of PEG400 is added to the solution of benzyl alcohol and budiodarone in order to prepare an isotonic injectable solution. The solution can be kept in a sterile container until it is diluted with 10 ml of water just before administration.

Preparation of Samples:

In each of three screw-cap glass vials, budiodarone tartrate (250 mg, 0.293 mmole) and benzyl alcohol (914 0.887 mmole) were mixed. In vial #1, 120 μl of propylene glycol (PG) was added. In vial #2, 150 μl of DMA was added. In vial #3, 582 μl of PEG400 was added. The vials were then vortexed at high speed to dissolve the solid. Gentle warming helped the dissolution process. In vial #3, the resulting mixture was turbid, indicating that the benzyl alcohol/budiodarone tartrate mixture was not fully soluble in the PEG400. The resulting oils were then kept in the refrigerator overnight and were diluted with water to a final volume of 10 ml. Dissolution was rapid and only necessitated a few seconds of agitation. All three vials appeared to contain a clear solution. Half of the solution in each vial (5 ml) was filtered through 200-nm-pored nylon membranes.

Samples were analyzed by HPLC as described in previous examples.

The results agreed with the predicted budiodarone concentration of 25 mg/ml. However, analysis of sample #3 shows a loss of material upon filtration.

Vial #1: budiodarone concentration is 25.1 mg/ml
Vial #1 filtered: budiodarone concentration is 25.2 mg/ml
Vial #2: budiodarone concentration is 26.6 mg/ml
Vial #2 filtered: budiodarone concentration is 27.7 mg/ml
Vial #3: budiodarone concentration is 26.3 mg/ml
Vial #3 filtered: budiodarone concentration is 21.3 mg/ml

The molecular complex of benzyl alcohol/budiodarone tartrate (3/1 mol/mol) was soluble in small volumes of co-solvents such as PG and DMA to form a solution. As a co-solvent, PEG400 resulted in lower solubility of budiodarone than co-solvents PG and DMA. The solutions were stable in the cold for some time.

The solution made in the presence of PG and DMA can be rapidly reconstituted to form an intravenous formulation that is isotonic to blood. The reconstituted intravenous formulation can be sterile-filtered through a 200-nm-pored membrane without any measurable loss of material.

### Example 12

### Identification of the Complex Made of Benzyl Alcohol and Budiodarone Using ¹H-NMR Spectroscopy

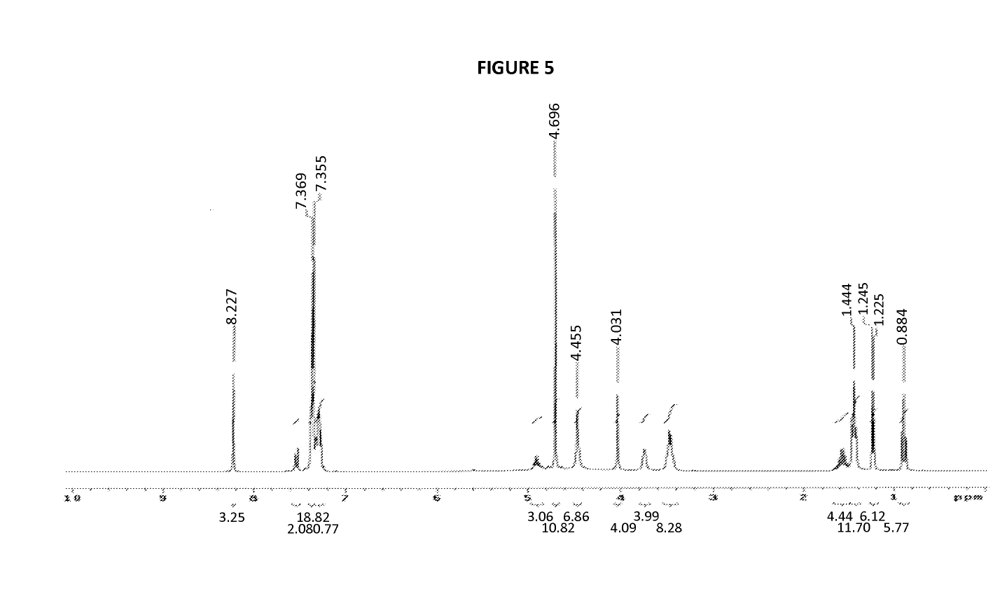

Three samples (samples number 173-23, 173-46, and 173-92) each containing 125 mg of budiodarone tartrate (0.146 mmole) and 23 μl, 46 μl, or 92 μl of benzyl alcohol (0.222, 0.444, or 0.888 mmole), respectively, were prepared. The sample were dissolved in 3 ml of CDCl₃ and analyzed by proton-NMR spectroscopy (FIG. 5).

The ratio of peak intensities at 4.70 ppm and at 8.21 ppm in the spectrum of FIG. 5 (Sample 173-46) were calculated as follows: 10.82/3.25=3.33. So, a ratio of 3.33/1 was calculation using NMR techniques, which was a good estimate of the actual ratio of 3.04/1. Similarly, for Sample 173-23, a calculated ratio was 6.52/4.12=1.58, which was good estimate of the actual ratio of 1.52/1. For Sample 173-92, a calculated ratio was 15.52/2.60=5.96, which was good estimate of the actual ratio of 6.08/1.

### Example 13

### Identification of the Complex Made of Benzyl Alcohol and Budiodarone Using HPLC

Three samples (Sample numbers 173-23, 173-46, and 173-92) were prepared, as described in Example 12. From each sample, an aliquot of 24 μl was taken and diluted with 1,976 μl of methanol. Each final sample contained 0.50 mg/ml of budiodarone tartrate and either 0.0092%, 0.0184%, or 0.0368% of benzyl alcohol, respectively.

These samples were then analyzed by HPLC using a reverse-phase chromatography column (Zorbax C₈-4.5×150 mm) according to the following conditions: The mobile phase was made of two solvents. Solvent A was 0.1% trifluoroacetic acid (TFA) in water and solvent B was 0.1% TFA in acetonitrile. A mixture of B/A=25:75 at 2.0 ml/min for 2 minutes followed by B/A=70/30 for 3 minutes at 2.0 ml/min was used, then B/A=25:75 for 2 minutes at 2.0 ml/min was used. The retention time of benzyl alcohol was 2.39 min and the retention time of budiodarone was 3.94 min. Benzyl alcohol and budiodarone concentrations were then extrapolated from standard concentration curves correlating concentration with the integrated area under the curve (AUC) of the chromatogram. These concentration curves were built from a series of samples of known concentrations of test compounds analyzed by HPLC under similar conditions. The results are shown in Table 12.

TABLE 12
Molar ratios of benzyl alcohol to budiodarone measured by HPLC in
samples number 173-23, 173-46, and 173-92.

| | | Benzyl | Molar |
|---|---|---|---|
| Sample number | Budiodarone tartrate (mg/ml) | alcohol (% v/v) | ratio |
| 173-23 | 0.48 | 0.0104 | 1.78 |
| 173-46 | 0.57 | 0.0223 | 3.22 |
| 173-92 | 0.58 | 0.0478 | 6.77 |

The molar ratio estimates in the three samples using HPLC technique are shown in Table 12. The three analytical samples were prepared in the same way as samples analyzed by NMR in Example 12. The combination of NMR and HPLC techniques to analyze a given sample is a reliable way to determine the molar ratio of benzyl alcohol and budiodarone in an unknown sample.

### Example 14

### Formulation of a Hyperconcentrated Solution Comprising 3:1 Benzyl Alcohol/Budiodarone Tartrate

A hyperconcentrated solution of benzyl alcohol and budiodarone tartrate in a 3/1 ratio in water was made and was stable at room temperature for several months. To 250 mg of budiodarone tartrate (0.293 mmole) was added 91 μL (0.859 mmole) of benzyl alcohol. The mixture was thoroughly mixed and to this was added 409 μL of water. The mixture was vortexed at maximum speed for 3×10 sec and then was sonicated for 30 seconds, resulting in a clear liquid containing approximately 250 mg of budiodarone tartrate in a volume a little over 0.5 ml (0.409 ml of water+0.091 ml of benzyl alcohol+volume due to budiodarone tartrate). This concentrated solution was then diluted to a final volume of 50 ml with D5W to make a 5 mg/ml solution of benzyl alcohol and budiodarone in a 3/1 ratio. This solution was analyzed by HPLC (Conditions 3) and the concentration of budiodarone in this diluted solution was found to be 5.5 mg/ml, which is within the margin of error of the HPLC method.

A hyperconcentrated solution of benzyl alcohol and budiodarone in a 3/1 ratio was made. This hyperconcentrated solution can be diluted with a non-ionic aqueous solvent such as D5W, for example in order to make an injectable solution.

### Example 15

### Amiodarone and Benzyl Alcohol does not Make a Constant Molar-Ratio Complex

This example demonstrates that the special properties of the benzyl alcohol/budiodarone mixture could not have been anticipated from prior experience with amiodarone, a very close structural analog to budiodarone. Unlike budiodarone, amiodarone does not make a constant molar-ratio complex with benzyl alcohol in the same way budiodarone does. In order to find out whether the spontaneous ratio of 5/1 between benzyl alcohol and budiodarone is a common phenomenon with other molecules of similar chemical structures, amiodarone was tested, similar to Examples 3 and 6. Using conditions similar to Example 3, amiodarone at 50 mg/ml concentration and benzyl alcohol at concentrations between 0.77% and 3.8% (corresponding to molar ratios of from 1/1 to 5/1) were tested. The benzyl alcohol concentrations used in this example were similar to the benzyl alcohol concentrations used in Example 3 in the flat portion of the 50 mg/ml curve.

Samples were prepared as indicated in Table 13 by repeating the experiment protocol described in Example 3. Water was added to amiodarone hydrochloride to make an aqueous suspension, then benzyl alcohol was added and the mixture was mixed by vortexing for 3×10 sec and then the samples were left overnight in the dark at room temperature.

TABLE 13
Preparation of 50 mg/ml amiodarone hydrochloride in 0.77% to 3.8%
benzyl alcohol

| | Amio. | | | Molar | |
|---|---|---|---|---|---|
| Vial # | HCl (mg) | Water (μl) | BnOH (μl) | Ratio | BnOH conc. (%) |
| 1 | 150 | 2,977 | 23 | 1/1 | 0.77 |
| 2 | 75 | 1,477 | 23 | 2/1 | 1.5 |
| 3 | 75 | 1,466 | 34 | 3/1 | 2.3 |
| 4 | 75 | 1,454 | 46 | 4/1 | 3.1 |
| 5 | 75 | 1,443 | 57 | 5/1 | 3.8 |

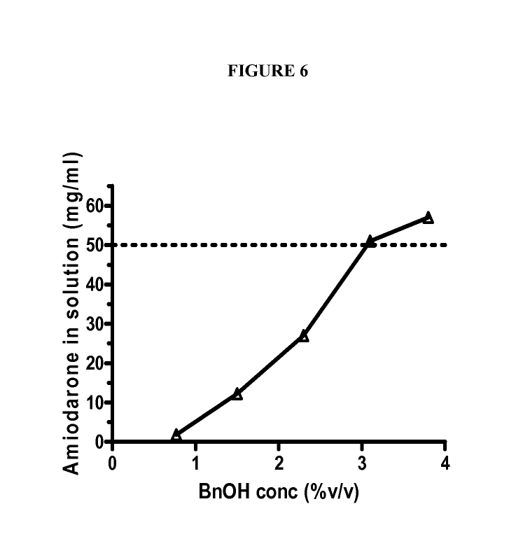

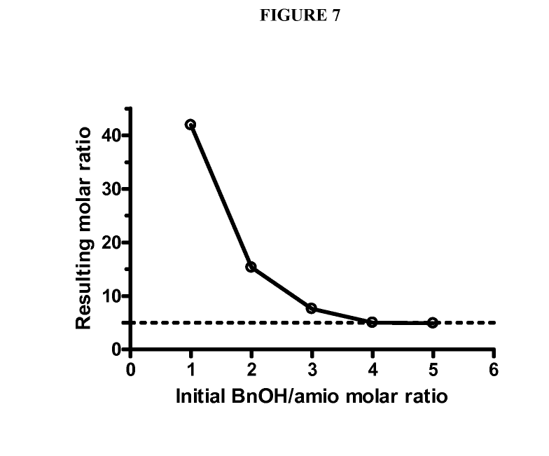

The results are shown in FIGS. 6 and 7. The first observation is that amiodarone's aqueous solubility, just like budiodarone, increases with higher amounts of benzyl alcohol in the sample (FIG. 6 vs FIG. 1). The results show that amiodarone hydrochloride becomes fully soluble at 50 mg/ml when the benzyl alcohol concentration is between 2.3 and 3.1% (not fully soluble at 2.3% but fully soluble at 3.1%).

Budiodarone makes a spontaneous constant molar ratio of 5/1 with benzyl alcohol at room temperature when the concentration of benzyl alcohol is limiting and budiodarone is in excess. This ratio does not go beyond 5/1 even when budiodarone is not fully soluble (FIG. 2 and FIG. 4). By contrast, amiodarone does not make a constant ratio with benzyl alcohol (FIG. 7). This is a difference between two molecules with very similar chemical structures (compare FIG. 7 and FIG. 4). These results demonstrate that the behavior of the solution of benzyl alcohol and budiodarone is unpredictable from the behavior of amiodarone under the same conditions.

### Example 16

### Budiodarone Concentration Attainable in a Hyperconcentrated Solution Containing a 3/1 Molar Ratio of Benzyl Alcohol to Budiodarone

The benzyl alcohol/budiodarone tartrate solution is viscous and therefore direct measurement of the budiodarone concentration is difficult. A better way to measure budiodarone concentration in the solution of benzyl alcohol and budiodarone oil or in a hyperconcentrated solution is by using an indirect method. In this example, the volume of the benzyl alcohol/budiodarone mixture was measured, which in turn allowed for the calculation of an accurate budiodarone concentration.

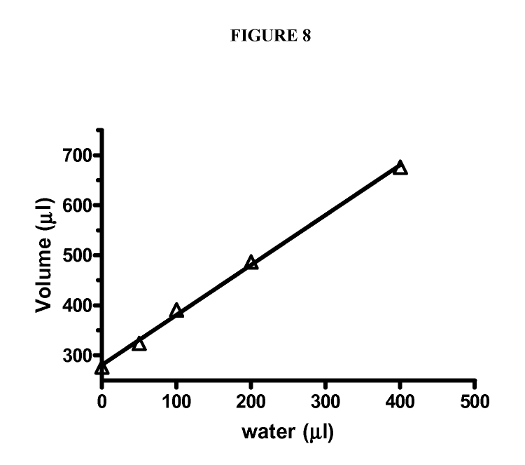

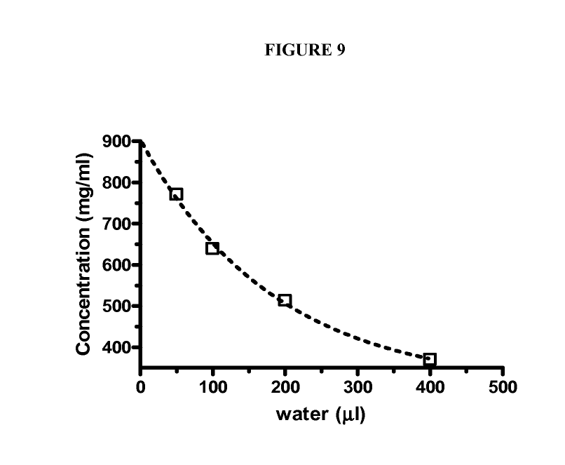

In each of five vials of accurately known volume, budiodarone tartrate (250 mg) and benzyl alcohol (91 μl) were mixed in order to make a 3/1 mixture (mol/mol). Then the solution was mixed thoroughly in order to make a clear solution. This was aided with the help of warming to 50° C. for 3 minutes in order to reduce the viscosity of the mixture. The weight of the vial containing the 3:1 mixture of benzyl alcohol/budiodarone was then recorded. Vial number 1 was filled with silicon oil of density d=0.964 and weighed again. The difference in weight provided the weight of the silicon oil added. This, divided by the density of the silicon oil provided the volume of silicon oil necessary to fill the vial. The difference with the known volume of the vial represents the volume of the benzyl alcohol/budiodarone mixture. To Vial numbers 2, 3, 4, and 5 was added 50, 100, 200, and 400 μl of water, respectively. The contents of the vials were then thoroughly mixed in order to get hyperconcentrated solutions of different volumes and different budiodarone concentrations. The volumes of these hyperconcentrations using silicon oil were measured as described above. The results are shown in FIGS. 8 and 9.

Linear regression analysis of the data gives the equation V=0.281+(0.999·W), where V is the volume of solution and W is the volume of water added in ml. FIG. 8 shows that the relation between the volume of water added to the benzyl alcohol/budiodarone mixture and the volume of the hyperconcentrated solution is linear with a slope of 0.999 (very close to 1), as expected. The goodness of fit of the data to the linear equation is r²=0.998, with a probability of error p<0.1%. The volume of the 3:1 mixture of benzyl alcohol/budiodarone is 0.281 ml (W=0), which corresponds to a budiodarone concentration of 890 mg/ml and a benzyl alcohol concentration of 32.4% (v/v). FIG. 9 shows the same results by plotting the concentration of the hyperconcentrated solutions as a function of the volume of water added. A non-linear regression analysis of the data gives a curve with equation y=251/(x+281), which is very close to the expected curve y=250/(x+281), 250 being the mg amount of budiodarone tartrate in each sample. We can therefore conclude that a calculated maximum concentration that can be attained in a hyperconcentrated aqueous solution of 3:1 molar ratio of benzyl alcohol/budiodarone is about 890 mg/ml, with a maximum benzyl alcohol concentration of 32.4% (v/v).

### Example 17

### Budiodarone Concentration Attainable in a Hyper-Concentrated Solution Containing a 5/1 Molar Ratio of Benzyl Alcohol/Budiodarone Formulation

The same experiment as Example 16 was replicated, using mixtures of 5/1 ratio (250 mg of budiodarone tartrate and 152 μl of benzyl alcohol) and various volumes of water added. Using the same procedure as Example 16, a maximum calculated budiodarone concentration of 737 mg/ml and a maximum benzyl alcohol concentration of 45% (v/v) were found.

### Example 18

### Budiodarone Concentration Attainable in a Hyper-Concentrated Solution Containing a 1/1 Molar Ratio of Benzyl Alcohol/Budiodarone Formulation

Similarly, the experiment in Example 16 was replicated, using a 1/1 mixture (250 mg of budiodarone tartrate and 30 μl of benzyl alcohol). A maximum calculated budiodarone concentration of 1,136 mg/ml and a maximum benzyl alcohol concentration of 13.6% (v/v) were found.

### Example 19

### Turbidity Measurement of Aqueous Solutions of Benzyl Alcohol and Budiodarone for Measuring Physical Stability in Solution

Turbidity of the aqueous solution can be measured over time in order to estimate how long an aqueous solution containing benzyl alcohol and budiodarone is physically stable. This can be performed by measuring the UV absorption of an aqueous solution of the oil at a wavelength of 550 nm. Both benzyl alcohol and budiodarone have negligible UV absorption at that wavelength, and any increase in UV absorption over time at 550 nm means that light is blocked or diffracted by particulates in suspension. The larger the particulates, the greater the absorption. In this example, aqueous solutions of benzyl alcohol and budiodarone tartrate will be be analyzed by UV spectroscopy at 550 nm wavelength and at various time points and will show that the physical stability of the aqueous solutions of benzyl alcohol and budiodarone at various ratios and various concentrations are stable over a length of time that is consistent with its use as a reconstituted injectable formulation.

For long-term stability in an aqueous solution, accelerated stability studies at controlled temperatures (6-month duration or longer) are predictive of long-term stability at room temperature. This can be evaluated using temperature-and-humidity-controlled incubators, usually at 3, and preferably at 4 different temperatures for periods of time as long as 6 months and sometimes longer.

### Example 20

### Light Diffraction Analysis for Measuring Physical Stability Over Time

Similarly, aqueous solutions of the oil can be analyzed using dynamic light scattering techniques such as photon correlation spectroscopy. This technique allows for the measurement of nano-sized particulates and a correlation between particle size over time can be made to estimate physical stability. If particle size increases over time, then eventually they come out of solution and the solution is not stable. In this example, aqueous solutions of benzyl alcohol and budiodarone tartrate will be analyzed by photon correlation at various time points and will show that the physical stability of the aqueous solutions of benzyl alcohol and budiodarone at various ratios and various concentrations are stable over a length of time that is consistent with its use as a reconstituted injectable formulation.

### Example 21

### Stability of the 5/1 Ratio of Benzyl Alcohol/Budiodarone at Room Temperature

Samples were prepared as in Table 14 below.

TABLE 14
Preparation of various concentrations of benzyl alcohol/
budiodarone 5/1 (mol/mol)

| | Sample # | | | |
|---|---|---|---|---|
| | 1 | 2 | 3 | 4 |
| Budiodarone (mg) | 125 | 250 | 250 | 250 |
| BnOH (μl) | 76 | 152 | 152 | 152 |
| Water (μl) | 12,424 | 9,848 | 4,848 | 2,348 |
| Budiodarone (mg/ml) | 10 | 25 | 50 | 100 |
| BnOH (%) | 0.61 | 1.52 | 3.04 | 6.1 |

Water was added to budiodarone in 20-ml screw-cap vials. Following mixing to make a suspension, benzyl alcohol was added and the mixture was vortexed as described in previous examples until a clear solution was formed. The solutions were then filtered through a 200-nm pored nylon membrane and the filtrate was kept a room temperature protected from direct light. Small aliquots of each sample were taken from time to time and diluted with methanol as follows: Sample #1 was diluted 20-fold, sample #2 was diluted 50-fold, sample #3 was diluted 100-fold, and sample #4 was diluted 200-fold. The resulting dilutions were then analyzed by HPLC as described in previous examples. The results are shown in Table 15.

TABLE 15
Stability of various solutions with 5/1 ratio at room temperature. Samples
are described in Table 14. The amount of budiodarone tartrate in
solution (mg/ml) is shown at various times Dx, where x is the number
of days following preparation of the sample.

| Sample # | D₀ | D₇ | D₃₁ |
|---|---|---|---|
| 1 | 9.9 | 11.6 | 9.8 |
| 2 | 26 | 28 | 26 |
| 3 | 53 | 57 | 53 |
| 4 | 108 | 115 | 100 |

The results at time D₀ show that the solutions (10, 25, 50, and 100 mg/ml) can be sterile filtered through 200-nm pored nylon membranes without any appreciable loss of compound. The results also show that the solutions are physically and chemically stable for at least 31 days at room temperature and away from direct light. In some embodiments, the solutions are physically and chemically stable for at least 57 days.

### Example 22

### Stability of the Hyperconcentrated Solution of Example 14 at Room Temperature

Budiodarone tartrate (500 mg) was mixed thoroughly with benzyl alcohol (182 μl) to make a thick oil in a 3/1 molar ratio. Water (818 μl) was added and the mixture was vortexed to make a clear solution. According to the equation of Example 16, the total volume of the solution was 1.380 ml and the budiodarone concentration was 500/1.38=362 mg/ml.

A small aliquot of the hyperconcentrated solution was diluted 1.000-fold with methanol and was analyzed by HPLC as described in Example 1. The results show that the budiodarone concentration is 364 mg/ml, which is close to the expected value. The hyperconcentrated solution was kept away from light at room temperature and was analyzed again at Day 38. The results at Day 38 showed a budiodarone concentration of 369 mg/ml. Therefore the hyperconcentrated solution is stable at room temperature for at least 38 days.

### Example 23

### Stability of the Solution of Benzyl Alcohol and Budiodarone at Room Temperature

Mixtures containing benzyl alcohol and budiodarone in various molar ratios of between 1/1 and 5/1 can be prepared as described in previous examples and can be kept at room temperature protected from light. At various time points up to several months, these samples can be reconstituted with water to make a 25 mg/ml concentration. The reconstituted solutions can then be centrifuged and the supernatant can be analyzed by HPLC as in Example 1.

### Example 24

### Formulation of Solution of Benzyl Alcohol and Budiodarone in 3:1 Ratio in Gelatin Capsules

Budiodarone tartrate (1,000 mg) and benzyl alcohol (0.364 ml) are mixed to make an oil with 3/1 ratio. This oil is put into a 1-ml graduated syringe, and a precisely measured aliquot of volume 0.280 ml is injected into size-3 hard-gelatin capsules.

The capsules are dissolved into 10 ml of warm water at 37° C. and the resulting solutions are stirred until homogeneous. A small aliquot (0.100 ml) is diluted 50-fold with methanol and is analyzed by HPLC. The results can show that the amount of budiodarone in the capsules is close to the expected amount of 250 mg.

### Example 25

### Formulation of Solution of Benzyl Alcohol and Budiodarone in 5:1 Ratio in Gelatin Capsules

Budiodarone tartrate (1,000 mg) and benzyl alcohol (0.607 ml) are mixed to make an oil with 5/1 ratio. This oil is put into a 1-ml graduated syringe, and precisely measured aliquots of volume 0.300 ml are injected into size-3 hard-gelatin capsules.

The capsules are dissolved into 10 ml of warm water at 37° C. and the resulting solutions are stirred until homogeneous. A small aliquot (0.100 ml) is diluted 50-fold with methanol and is analyzed by HPLC. The results can show that the amount of budiodarone in the capsules is close to the expected amount of 250 mg.

### Example 26

### Other Oral Formulations of Solution of Benzyl Alcohol and Budiodarone

Solutions of benzyl alcohol and budiodarone of ratios between 1/1 and 5/1 are dissolved into a concentrated sugar solution, such as 70% sorbitol, to produce a pharmaceutical syrup for oral administration.

Several syrups of various budiodarone concentrations can be made. The budiodarone concentrations in the syrup can be between 25 mg/ml and 250 mg/ml. Alternatively, the benzyl alcohol/budiodarone mixtures of ratios between 1/1 and 5/1 is mixed with inert fillers such as cellulose esters, microcrystalline cellulose, or other excipients commonly used in solid oral dosage form preparations to make a solid dosage form that can be compressed into a pellet. The unit doses of budiodarone tartrate in these pellets can be between 50 mg and 800 mg.

### Example 27

### Improved Oral Bioavailability of Solution of Benzyl Alcohol and Budiodarone

In a typical experiment, solutions of benzyl alcohol and budiodarone of ratios from 1/1 to 5/1 are prepared as described in examples above. They are placed directly into hard gelatin capsules. One group of Beagle dogs (n=5) receives an oral dose of 10 mg/kg of the oily solution inside a gelatin capsule, and blood samples are taken from the brachial vein at various time points up to 24 hours post dosing. The plasma is separated by centrifugation and the proteins are precipitated with acetonitrile. The acetonitrile extract is analyzed by HPLC following one of the methods described in Example 1. Plotting the plasma concentrations as a function of time post-dosing allows for the calculation of the area under the curve (AUC), the maximal plasma concentration (Cmax), and the time to achieve Cmax (Tmax). The same experiment is repeated with the same group of dogs after 1 week in order to ensure that the drug has had time to wash out. This second time, the same group of dogs receives 10 mg/kg orally of budiodarone tartrate powder (no benzyl alcohol) in gelatin capsules. An AUC, Cmax, and Tmax are computed as before, and the results of the two experiments are compared. The results will show that when the dogs receive a solution of benzyl alcohol and budiodarone, their AUC and Cmax is higher than when they receive budiodarone tartrate as a powder with benzyl alcohol.

### Examples of Testing of Budiodarone in Patients

Examples of testing of budiodarone can be found in Arya A, et al., Europace. 2009 April; 11(4):458-64. Epub 2009 Jan 26 and Ezekowitz M et al., PASCAL: a randomized double-blind, placebo-controlled study of budiodarone (ATI-2042) in patients with paroxysmal atrial fibrillation and pacemakers with atrial fibrillation data logging capabilities [abstract], presented at Heart Rhythm Society Annual Scientific Sessions, Boston, Mass., May 2009, which are hereby incorporated by reference in their entireties.

### Example A

### Budiodarone (ATI-2042) and AF

Example A is adapted from US Publication No. 2011/0136779.

The primary objective of the study was to assess the efficacy of budiodarone, (S)-sec-butyl 2-(3-(4-(2-(diethylamino)ethoxy)-3,5-diiodobenzoyl)benzofuran-2-yl)acetate, in treating AF, as measured by a reduction in AF burden (AFB) in subjects with paroxysmal atrial fibrillation who had implanted pacemakers (Arya A, et al., Europace. 2009 April; 11(4):458-64. Epub 2009 Jan 26).

This study was a proof of concept design seeking preliminary information on the pharmacodynamic effects, safety, and tolerability of the investigational drug ATI-2042 at a variety of doses, in patients with PAF. Patients with advanced DDDRP pacemakers were selected because of the pacemaker's sophisticated diagnostics and the ability to record continuously and log asymptomatic as well as symptomatic episodes.

The molecular structure of budiodarone is identical to that of amiodarone, except for the presence of a sec-butyl acetate side chain at position 2 of the benzofuran moiety. The core of the molecule is a benzofuranyl ring system, to which an iodinated diiodophenyl group, a tertiary amine, and the chiral centre of the molecule, an (S)-2-butanyl group, are added over the course of the synthesis. The final drug substance is provided as a tartrate salt.

ATI-2042 is not a prodrug of amiodarone, nor is amiodarone a metabolite of ATI-2042. The electrophysiological activity of ATI-2042 in animals includes inhibition comparable with amiodarone of sodium, potassium, and calcium ion channels, increased left and right atrial refractoriness comparable with amiodarone, atrial effects (increased St-A and A-H intervals), and ventricular effects (increased MAPD90 and QT-interval). The major metabolite (ATI-2000) is electrophysiologically inactive.

Only post-menopausal or surgically sterile females with a significant PAF burden and pacemakers were included in this study. The pacemakers had to have been in situ for at least 1 month prior to the study and have appropriate arrhythmia diagnostics. In this study, Vitatron pacemakers, models Selection 9000 or T70 pacemakers, were used. Non-specific but potentially toxic findings were observed in canine testes during pre-clinical safety testing. Although this finding was explored further, it was prudent to commence clinical testing in a population not at risk for this effect. Hence, the study described in Example 1 was limited to post-menopausal or surgically sterile females; the use of the drug in males has been addressed in the study described in Example B.

The patients underwent screening assessments to assess suitability for the study and to obtain a baseline medical history and examination. The value of this cohort was that the pacemaker was not inserted for bradycardia but for the treatment of AF using various prevention pacing and rate control techniques. The inclusion criteria were as follows: age 18-85 years; AF burden (AFB) of 1-50%; able to have pacemaker anti-arrhythmic algorithms turned off or remain at a stable setting; stable warfarin regimen; be generally healthy and free from significant comorbid illnesses; and able to understand study requirements.

The exclusion criteria were significant structural heart disease (ejection fraction <45% and congestive heart failure); abnormal QTc interval (i.e. >470 ms); an abnormal 12-lead electrocardiogram (ECG); known hypersensitivity to amiodarone or iodine; chronic treatment with amiodarone within 3 months; demonstrated lack of efficacy with amiodarone treatment; treatment with any other investigational drug within 30 days; treatment with any anti-arrhythmic medication (exclusive of a stable dose of digoxin or a beta-blocker or calcium blocker) within five half-lives prior to study entry; major surgery within 3 months prior to study entry or any surgery within 2 weeks prior to study entry; or any laboratory assay result that was out of the normal reference range at screening from a standard battery of blood chemistry, haematology, and urinalysis tests. Patients were enrolled within 8 weeks of screening. During the study, they were reviewed on days 1, 2, and 8 of each study period. ATI-2042 was increased on day 1 of each 2-week study period, following routine bloods for haematology, biochemistry, and coagulation screens. Plasma samples for pharmacokinetic analysis of ATI-2042 and its metabolites were taken at steady state at the end of each study period just prior to the first escalated dose of the subsequent period and within 15 min prior to the pre-dose ECG for that dose.

Patients were then monitored for at least 3 h continuously post-dose; this included telemetry, vital signs, and oxygen saturations. Electrocardiograms were taken, and the pacemaker data were downloaded prior to drug administration on day 8 and day 14 of each study period.

Criteria for drug discontinuation included a fall in systolic blood pressure (BP) to <90 mmHg systolic, an increase in BP>200 mmHg, intolerable side-effects, a change in rhythm that in the opinion of the investigator constituted a risk to safety, or a QRS increase >50%. Where possible, ECGs of intrinsic rhythm rather than ventricular-paced rhythm were obtained, as paced complexes can be difficult to interpret for QT prolongation. An increase in QT interval >470 ms for intrinsic and >550 ms for paced beats or an increase of 30% was considered significant.

The study consisted of six 2-week periods: a baseline period (p1), four treatment periods (p2-p5), and a washout period where return to baseline was observed (p6).

The initial ATI-2042 dosage for all subjects was 200 mg orally bid, and it was then increased by 200 mg bid for each subsequent study period. Patients received 200 mg bid of ATI-2042 during period 2, 400 mg bid during period 3, 600 mg bid during period 4 and 800 mg bid during period 5, and no drug was administered during baseline and washout periods.

“Selection 9000” and “T70” pacemakers (Vitatron, Arnhem, The Netherlands) are dual-chamber pacemakers with sophisticated and similar algorithms for AF detection and prevention. Atrial fibrillation detection is based on atrial rate; atrial tachyarrhythmias are detected when the median atrial cycle length is less than that programmed for AT or AF detection. In all patients, atrial fibrillation was detected if the atrial rate was >200 bpm for six consecutive beats, and its end logged if the atrial rate dropped below 200 bpm for 10 beats. An arrhythmia diary of up to 400 episodes and 15 detailed onset reports (DORs) were recorded with rate profile, interval plots, and electrograms to confirm diagnosis. Pacemaker anti-arrhythmic algorithms were turned off prior to entry into the baseline period and remained turned off until after washout.

Pacemaker data, for the primary outcome measure AFB, were downloaded on days 8 and 14 of each 2-week period to allow up to 800 episodes of AF to be recorded.

The primary outcome measure, AFB, was defined as the duration of time the subject's cardiac rhythm was AF divided by the total time recorded for that study period, expressed as percent. The total duration of time that the rhythm was AF is a function of the number of PAF episodes and the duration of each episode. Therefore, a reduction in AFB can occur through reduction in either or both of these variables. Atrial fibrillation burden was compared with baseline during the treatment periods. Secondary outcome measures were the number of AF episodes, the safety of ATI-2042, and the incidence and severity of adverse events (AEs).

The minimum study interval was 2 weeks; data were acquired from the pacemaker and averaged to give a final value. Atrial fibrillation burden is given as the percentage of total storage duration.

The sample size for this study was selected empirically. All patients who received any amount of study medication were included in the efficacy and safety analysis. Efficacy variables for the study group are described as mean and standard deviation for each study period. Due to the small number of subjects, comparisons of periods 2-6 with baseline were made using estimates from a mixed-effects regression model. This model had a fixed, categorical effect of period and a random patient effect to account for correlations over time. A P-value of <0.05 was considered significant.

Electrocardiogram parameters (ventricular HR, PR, QRS, QT, QTc interval) were summarized by baseline, dosing period, and washout using descriptive statistics. Changes from baseline in ECG values at day 8 of dosing and day 14 of washout were also summarized. Baseline values of ECG parameters are defined as the mean of three values recorded prior to the first dose of ATI-2042.

An AE was defined as any untoward medical occurrence in a study subject administered a medicinal product (either study drug or marketed product), whether or not the event had a causal relationship with this product.

Trough concentrations of ATI-2042 and its metabolites (ATI-2000, ATI-2100, and ATI-2142), measured at pre-dose on day 1 of each of the four treatment periods and on days 1 and 8 of the washout period, were summarized by time point. Spearman's rank correlation was used to examine the relationship between trough concentrations of ATI-2042 and AFB.

Levels of pacemaker malsensing of AF or over- and under-sensing were evaluated by the manual examination of each DOR; this was performed for all patients and or every pacemaker download throughout the study and confirmed by an independent observer.

Six females, mean age (SD) 70.8+7.1 years with PAF of mean duration 4.7+2.3 years, were recruited. One patient withdrew in period 3 due to gastric AEs (nausea, flatulence, and loose stools) and for logistical reasons.

Patients were treated with a mean of 1.8+1.0 AADs, range 1-3, for PAF prior to study entry. Three patients had Vitatron T70 pacemakers and three had Selection 9000 s. All patients had echocardiographic assessments prior to the study; mean (SD) left atrial diameter 3.66+0.54 cm and mean fractional shortening 41.4+10.7%.

All patients were compliant with study medication. Mean trough levels of ATI-2042 were 0.0+0.0 ng/mL at baseline, 2.4+0.9 ng/mL at 200 mg bid, 5.2+1.7 ng/mL at 400 mg bid, 13.1+5.6 ng/mL at 600 mg bid, and 19.8+17.9 ng/mL at 800 mg bid, indicating some dose proportionality. In washout, trough levels of ATI-2042 were 0.3+0.4 ng/mL, and its metabolites were low or undetectable.

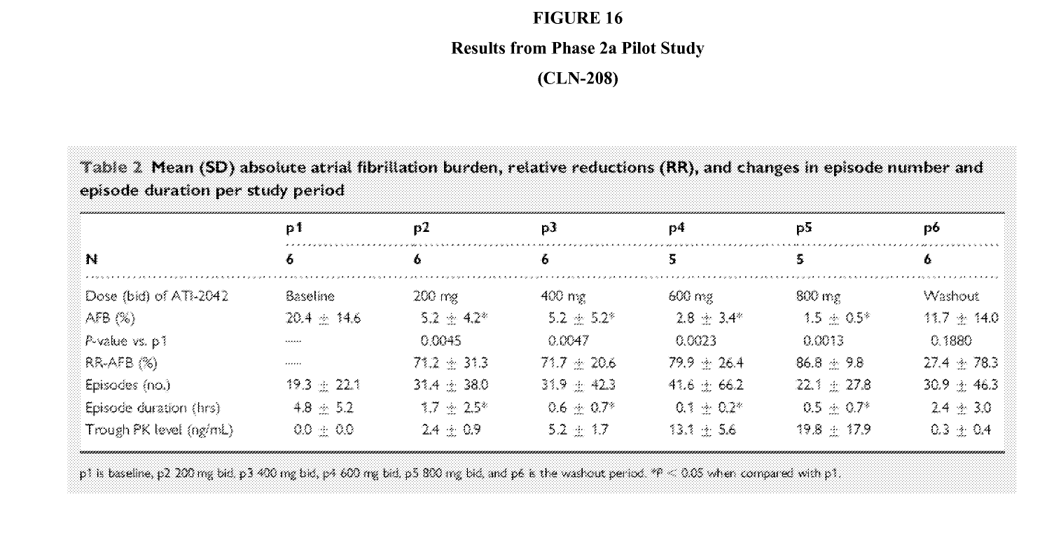

A summary of ATI-2042 efficacy measures is shown in FIG. 16. Mean AFB at baseline ranged from 4.6 to 45.3%, mean (SD) 20.3+14.6%. Absolute values of AFB decreased between baseline and all doses; mean AFB (SD) at 200 mg bid was 5.2+4.2%, at 400 mg bid 5.2+5.2%, at 600 mg bid 2.8+3.4%, and at 800 mg bid 1.5+0.5%. There was a 71.2+31.3% relative reduction (RR) in p2 from baseline (P=0.0045), 71.7+20.6% in p3 (P=0.0047), 79.9+26.4% in p4 (P=0.0023), and 86.8+9.8% in p5 (P=0.0013). Atrial fibrillation burden increased towards baseline in washout; mean (SD) 11.7+14.0%, range 0.8-38.4 (P=0.1880 compared with baseline).

The number of AF episodes increased initially with ATI-2042 and remained elevated in washout (FIG. 16). Mean episode duration (SD) decreased from baseline at 4.8+5.2 to 1.7+2.5 h in p2, to 0.6+0.7 h in p3, to 0.1+0.2 h in p4, to 0.5+0.7 h in p5. Mean episode duration increased in washout to 2.4+3.0 h, but did not reach baseline values.

There were no significant changes in HR, QRS, QT, or QTc between baseline and dosing or washout. There were no clinically significant changes in overall ECG interpretation. The PR interval showed a trend towards decreasing: reductions from baseline were 16.6+23.9% at 200 mg bid, 11.4+22.6% at 400 mg bid, 27.0+32.1% at 600 mg bid, 35.1+30.9% at 800 mg bid, and 30.7+24.3% at washout. There were no group or individual trends to QT or QTc prolongation with dosing. One patient exhibited a 30-60 ms change in QTc from baseline at 400 mg bid; this patient had had a previous atrioventricular (AV) node ablation and had permanently paced rhythm. A change in the paced QTc amounted to 15% from baseline. No patient exhibited >30% change from baseline QT or QTc with dosing, despite the presence or absence of pacing.

The drug was generally well tolerated. There were no serious AEs related to study drug. The number of subjects with AEs was similar in all groups, and most were of mild severity. The highest number of AEs was in period 5 (800 mg bid) and the fewest in period 4 (600 mg bid); gastric AEs, including transient nausea, flatulence, and loose stools, were more prevalent at 800 mg bid, clinically insignificant biochemical abnormalities at 400 mg bid, and cardiac AEs (transient palpitations) while taking 200 mg bid of ATI-2042.

There were no cases of proarrhythmia, clinical hypothyroidism, or hyperthyroidism. Three patients demonstrated dose-responsive increases in thyroid-stimulating hormone, which were outside the normal range. The level in one patient increased from 2.53 to 4.82 mU/L (normal range 0.27-4.2), another increased from 3.51 to 9.49 mU/L, and a third increased from 0.68 to 16.12 mU/L. None was associated with clinical abnormalities, and all returned towards normal after drug discontinuation in washout. There were minor fluctuations in free T4 and free T3, which were felt not to be clinically significant.

A total of 524 DORs were manually overread for accuracy, mean 87+69 per patient. A total of 10.9% of the DORs exhibited under- or over-sensing, and 6.3% of the DORs were undersensed almost exclusively due to blanking of P waves during AV delay as opposed to P wave fallout. Over-sensing was entirely due to farfield R sensing (4.6%) and was present in one patient; this patient had an excessive inter-electrode distance of 17 mm on the atrial pacing lead. The mean number of malsensed DORs/patient was 10+12.

During anti-arrhythmic drug (AAD) development, establishing human drug efficacy in phase I to III studies is often hindered by problems of proarrhythmia and tolerability. In addition, arrhythmic conditions are challenging to treat and evaluate because of heterogeneous temporal patterns of arrhythmia behavior.

This study was novel in using the sophisticated data logs of pacemakers to monitor drug efficacy continuously throughout the study and to record all episodes of AF including those that were asymptomatic. Patients with refractory PAF, i.e., those who had failed at least one AAD therapy were included in this study. ATI-2042 was significantly effective in reducing AFB at all doses in this group of patients. The endpoint of AFB can be affected by a reduction in the number of episodes of AF, indicating an effect on AF initiation, or by a reduction in the duration of episodes, indicating an effect on the sustainability of the episodes. In this study, therapy with ATI-2042 was associated with a mild trend for the number of episodes to increase with doses up to 600 mg bid, but this was offset by a substantial shortening of mean AF episode duration at all doses that reached statistical significance. The overall effect was a clinically and statistically significant reduction in AFB.

It was also apparent that the effects of ATI-2042, despite its short half-life, have a prolonged cardiac effect after discontinuation of the drug. Even in washout, AF parameters did not completely return to baseline. Trough levels of ATI-2042 and its metabolite were low or negligible within days of drug discontinuation, making drug persistence unlikely. Results indicate that even relatively short courses of this drug may promote atrial reverse-remodeling, which have a carry-over effect longer than its metabolism.

Overall, the drug was well tolerated. The absence of the electrocardiographic changes that were seen in animal testing may be due to the small sample size and/or the inclusion of patients with prior AV nodal ablation procedures with paced rhythms. One patient withdrew due to moderate gastric side effects and logistical reasons. There were no serious AEs related to study drug and no cases of proarrhythmia. Minor changes in thyroid function studies were likely reflective of the iodine content of ATI-2042. These resolved during continued study drug administration or after discontinuing the study medication. This pattern of thyroid function study changes is consistent with those reported for amiodarone. These findings require additional evaluation in future studies.

Paroxysmal atrial fibrillation is a common, distressing arrhythmia, which is often difficult to treat due to its heterogeneity and the tendency for AADs with class III action to exhibit reverse-use dependency. Drugs with multiple classes of action rather than specific class action, such as amiodarone, are the most efficacious in treating AF, but many drugs are limited to low-risk patients because of concerns regarding proarrhythmia. Amiodarone has been shown to be superior to other AADs in the maintenance of sinus rhythm post-cardioversion, but it is less effective in preventing recurrence in PAF than chronic AF. It has a pharmacokinetic and metabolic profile that contributes to its slow onset and offset of action and its toxicity. Prescribing class I agents, such as flecanide and quinidine, tends to be limited to patients without ischemic heart disease, who have preserved left ventricular function. This is due to the observation of increased mortality of post-myocardial infarction patients in the Cardiac Arrhythmia Suppression Trial (CAST; Echt D S, et al. “Mortality and morbidity in patients receiving encamide, flecamide or placebo: the CardiacArrhythmia Suppression Trial.”

NEnglJMed 1991; 324:781-8) and concerns regarding Torsades de Pointes extending to ‘pure’ class III agents such as dofetilide and ibutilide. New atrio-selective drugs that prolong atrial refractoriness without significant effects on ventricular refractoriness or the QT interval appear promising, but are early in development. In the present study, ATI-2042 was well tolerated and effective in reducing AFB, with decreases of at least 70% in AFB at all doses. Its short half-life, rapid onset and offset, small volume of distribution, and cytochrome P450-independent elimination represent attractive drug features of an AAD.

This study used the sophisticated monitoring capacity of pacemakers to record all episodes of AF and differed from the conventional means of assessing drug efficacy by the ‘time to first recurrence’ of AF. ‘Time to first recurrence’ is the time taken for an atrial tachyarrhythmia to recur post-chemical or electrical cardioversion. This measure makes the assumption that AF episodes are uniformly random, i.e. the risk of having an episode at any given time is uniform. However, recent data from pacemaker and defibrillator studies suggest a tendency to clustering of fibrillation episodes with the highest instantaneous risk of AF being immediately after termination. Human arrhythmia patterns vary between patients and the majority of episodes are asymptomatic, making assessment of drug efficacy in PAF patients challenging even with frequent study follow-up or trans-telephonic monitoring. Despite the complexities and heterogeneous nature of PAF, we propose that pacemaker data logs provide a comprehensive documentation of arrhythmia events. The degree of accuracy of the pacemaker diagnostics as determined by manual overreading supports the use of this method to measure AFB. Pacemaker logs may also monitor for proarrhythmia and can be used with handheld activators to correlate symptoms with events.

This study suggests that ATI-2042 is safe, well tolerated and may reduce AFB in patients with PAF. It has a promising electrophysiological and pharmacokinetic profile that makes it an attractive alternative to amiodarone. This study provides support for further clinical trials that evaluate the use of this investigational drug in an expanded cohort of patients with PAF and supports the concept of using implanted pacemaker devices to monitor AAD efficacy. Such a randomized, double-blind, placebo-controlled clinical trial of ATI-2042 is described in Example 2.

### Example B

### Budiodarone (ATI-2042) and AF, Round 2

Example B is adapted from US Publication No. 2011/0136779.

The objective of the study is to determine the efficacy of budiodarone in reducing atrial tachyarrhythmia (AT/AF) burden in patients with paroxysmal atrial fibrillation (PAF) compared to placebo, for 12 weeks of treatment, and the safety and tolerability of budiodarone for up to 12 weeks of treatment.

Secondary: to study the effect of budiodarone versus placebo on the number and duration of AT/AF episodes, duration of normal sinus rhythm (NSR) between episodes of AT/AF and on symptoms associated with PAF.

Example B describes a multicenter, multinational, randomized, double-blind, placebo-controlled, parallel-group study of the efficacy and safety of budiodarone in patients with PAF. Planned enrollment was up to 140 patients (with eventually 110 enrolled) with proven PAF who had permanently implanted pacemakers with appropriate AT/AF diagnostic and recording capabilities. Potential study participants underwent screening assessments, including the optimization of pacemaker programming for accurate AT/AF detection. Within 30 days after screening assessments began, eligible patients entered a 4-week baseline period (Period 1) when baseline atrial fibrillation burden (AFB) was established followed by randomization to one of three active treatment regimens or placebo for a 12-week treatment period (Period 2), followed by a 4-week washout period (Period 3). During the treatment period, patients received twice-daily (BID) oral doses of 200 mg ATI-2042, 400 mg ATI-2042, 600 mg ATI-2042, or placebo. No study drug was given during the baseline and washout periods.

110 patients were enrolled. Of 72 randomized and analyzed: 72 were treated and included in the intent-to-treat (ITT) and safety population; 60 were in the modified ITT (mITT) population; and 45 in the efficacy evaluable (EE) population.

Inclusion Criteria were the following: age 18 and above; proven PAF (electrocardiogram, Holter monitor, or pacemaker diagnosis obtained by the clinical site or patient's prior medical record documenting clear evidence of a diagnosis of PAF); pacemaker with appropriate AF diagnostic and recording capabilities implanted for at least 6 weeks (additional pacemaker requirements included: dual chamber with bipolar leads, able to diagnose and log AT/AF events, able to have AT/AF treatment algorithms turned off, capable of storing at least 4 weeks AT/AF data between downloads, and able to record and store electrograms); atrial P waves of adequate amplitude to allow accurate sensing and assessment of AT/AF episodes and no obvious indications of frequent oversensing or undersensing; Able to have pacemaker AT/AF treatment algorithms turned off for the duration of the study; able to understand study requirements and willing to follow instructions, attend all required study visits, and undergo all planned tests; women: unable to bear children, that is, post-menopausal (absence of vaginal bleeding or spotting) for at least one year or surgically sterile; men: starting at the time of study drug administration until completion of the 12-week treatment period, must have been willing to use an approved method of contraception (which included use of a condom with spermicide or use by partner of oral, implantable, or injectable contraceptives, intrauterine device (IUD), diaphragm with spermicide) or had a sterile sex partner.

To be randomized, study participant must have had: an AT/AF burden between 5% and 70% during the baseline period; no evidence of persistent AF (i.e., 7 or more consecutive days of AT/AF with episodes lasting >23 hours); able to have pacemaker atrial antitachyarrhythmia treatment algorithms turned off for the remaining duration of the study.

Efficacy outcome measures were AT/AF burden (total time spent in AT/AF as a percentage of total observation time), number and mean duration of AT/AF episodes, mean duration of NSR, patient global clinical impression (GCI) questionnaire, and University of Toronto Atrial Fibrillation Severity Scale (AFSS).

Safety: treatment-emergent adverse event (TEAE) type, severity, and incidence; clinical laboratory assessments, including thyroid function and testicular function (males); coagulation tests; vital signs; physical examinations; ECGs; eye examinations; chest X-rays; and pulmonary function tests.

Three analysis populations were used, which were: intent to treat (ITT; all randomized patients who had at least one assessment of AT/AF burden during the treatment period); modified intent to treat (mitt; all randomized patients who had a baseline AT/AF burden >3%, completed at least the first 4 weeks of treatment, and had been assessed as usable by the core lab and/or had over/undersensing detected by core lab adjudication but no gross violations of pacemaker programming guidelines); and efficacy evaluable (EE; all randomized patients who had a baseline AT/AF burden of 3% to 70%, completed at least the first 4 weeks of treatment, and had been assessed as usable by the core lab).

Statistical methods for efficacy: The primary efficacy analysis was the percent change in the AT/AF burden from baseline to the 12-week treatment period. Pairwise comparisons between each ATI-2042 dose group and the placebo group were performed using the Wilcoxon rank sum test. The primary analysis was based on the mITT population.

For all variables based on the pacemaker data, the Wilcoxon rank sum test was used to compare each ATI-2042 dose group to the placebo group. The Jonckheere-Terpstra test was used to test for a dose response. The Wilcoxon signed rank test was used to test for a significant change from baseline within each treatment group. An analysis of covariance (ANCOVA) was used to compare the percent change from baseline in AT/AF burden among the treatment groups after adjusting for the baseline AT/AF burden. The ANCOVA model contained effects for treatment group and baseline AT/AF burden.

For patient GCI, the Cochran-Mantel-Haenszel test was used to compare each ATI-2042 dose group to the placebo group. The Jonckheere-Terpstra test was used to test for a dose response.

Statistical methods for safety: Adverse events (AEs) were summarized by system organ class and preferred term. Laboratory parameters and vital signs were summarized by descriptive statistics and shift tables were also created for laboratory parameters. The percent of patients with physical examination findings that changed over the study, with ECG abnormalities by visit, and with corneal deposits were summarized. Chest X-rays and pulmonary function test results were provided in listings.

The ITT/safety population included 72 treated patients. Among the treated patients, 18 were randomized to placebo, 21 to 200 mg BID, 18 to 400 mg BID, and 15 to 600 mg BID. The mITT population was comprised of 60 patients and 45 patients comprised the EE population. Overall, 84.7% of the treated patients completed the study. The average age was 69.2 years (range, 51 to 88) and 98.6% of patients were white. Males comprised 56.9% and females 43.1% of the treated patients. Overall medical and cardiovascular history did not show notable differences among treatment groups. The mean AT/AF burden at screening was comparable across treatment groups and ranges from 17.8% to 23.4%.

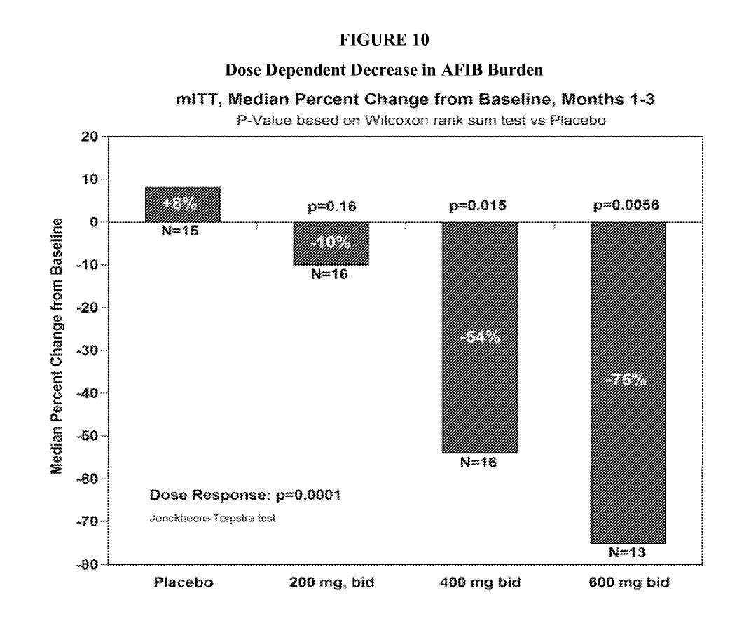

ATI-2042 at 400 mg BID and 600 mg BID achieved a significant reduction in AT/AF burden compared to placebo over Treatment Months 1-3. The median percent reductions from baseline were 54% (p=0.015) and 75% (p=0.006) for the 400 mg BID and 600 mg BID groups, respectively, in the mITT population, and 54% (p=0.013) and 74% (p=0.001) in the ITT population (FIG. 10). A significant dose response (p<0.0001) was seen for the primary endpoint of the study for both the mITT and ITT populations. The reduction in AT/AF burden was statistically significant in each of the 3 months of treatment in both the 400 mg BID and 600 mg BID groups. As the dose and treatment duration increased the effect became more pronounced. The maximal effect was seen at Treatment Month 3 (16 weeks) for the 600 mg BID group, with a median percent reduction of 83% (p=0.010) in the mITT population and 80% (p=0.002) in the ITT population.

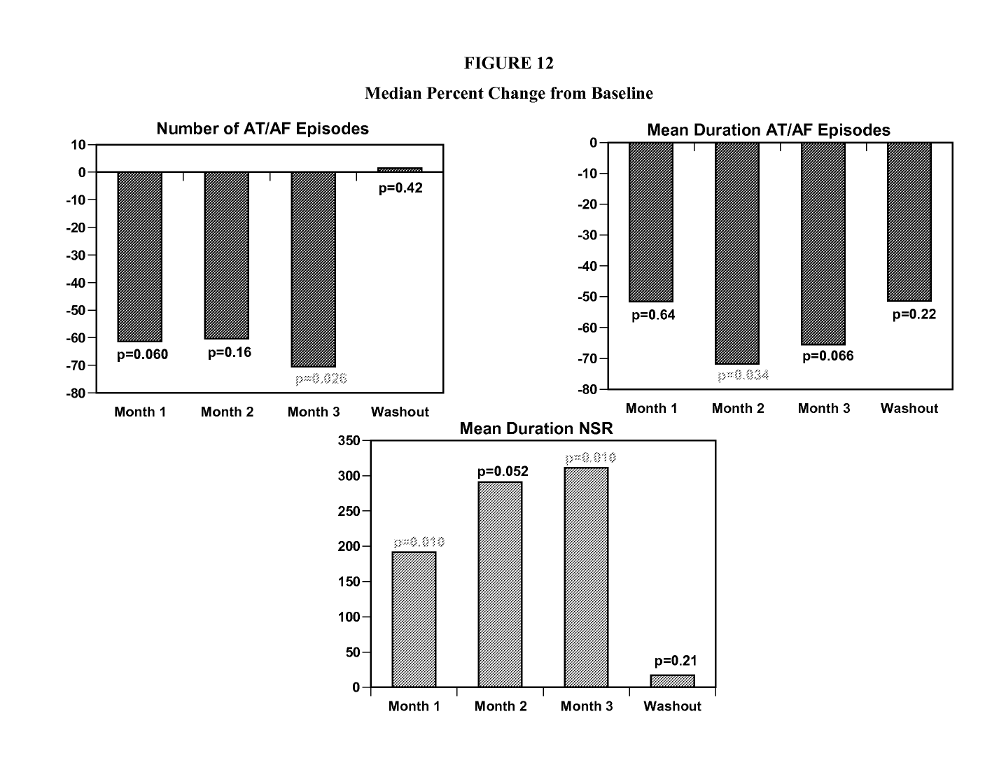

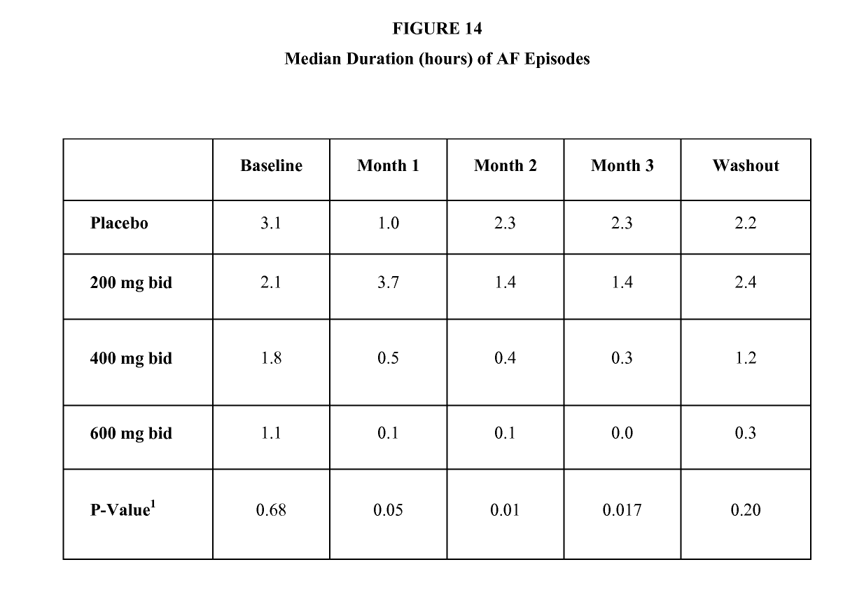

All secondary endpoints showed statistically significant improvement over Treatment Months 1-3 with 600 mg BID as shown by median percent changes from baseline in the number of AT/AF episodes (−62.1% in the mITT and −52.9% in the ITT population), duration of AT/AF episodes (−51.3% in the mITT population and −51.3% in the ITT population) and an increase in the duration of NSR (241.8% in the mITT population and 208.8% in the ITT population). See FIG. 12 through FIG. 15.

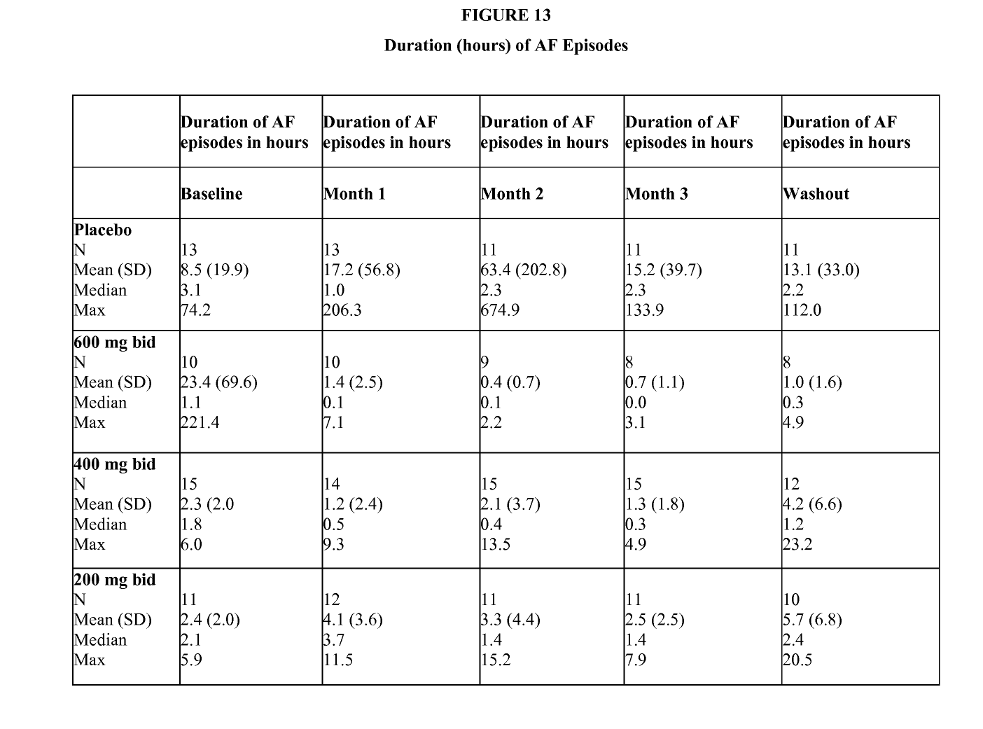

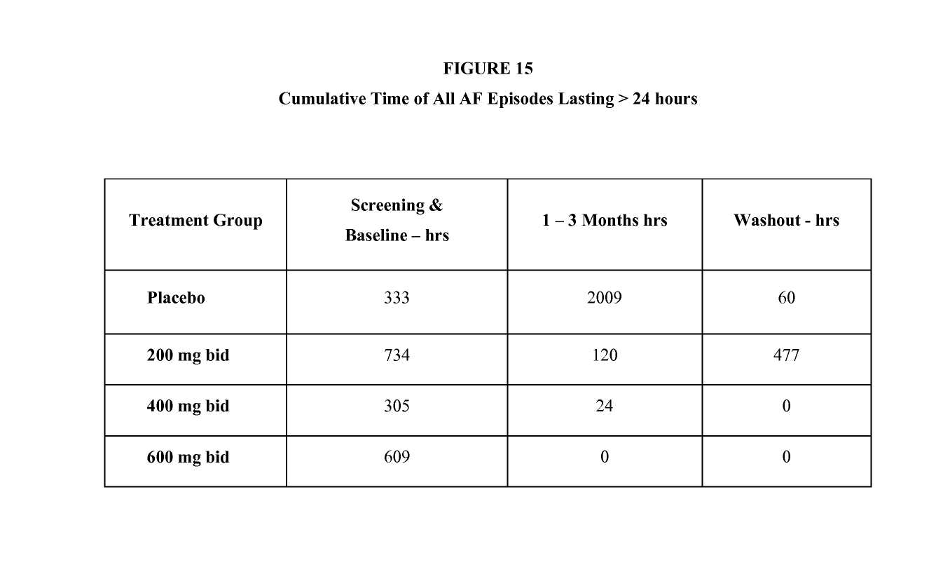

These are profound results that demonstrate how budiodarone can prevent or reverse atrial remodeling and reduce stroke risk. As mentioned above, AF is progressive, with PAF evolving to persistent and eventually permanent AF—a progression that occurs and accelerates with greater time spent in AF. That is, what is initially electrical and thrombotic remodeling on the hours and days timescale, eventually leads to structural remodeling on the months and years timescale, and with greater structural remodeling and greater electrical remodeling comes greater resistance to cardioversion. FIG. 12 shows that number and duration of AF episodes were reduced about 70% below baseline for patients on drug 600 mg drug. FIG. 13 shows that on 600 mg drug, patients' mean episode duration was reduced to less than 1 hour (down from nearly 24), and at month 3, the median AF episode duration was 0. More than half the patients had their AF eradicated during the study period. FIG. 15 shows that for the 600 mg dose group, no patients experienced an AF episode lasting 24 or more hours. The 400 mg dose cohort experienced only a single 24 hour episode. Less time in AF, particularly zero or only sporadic episodes of less than an hour, is much less likely to cause electrical, thrombotic and structural remodeling. Moreover, the concomitant increase in time in NSR enables the reversal of atrial remodeling. Thus, progression of AF should be retarded, halted or reversed, and stroke risk reduced. The concomitant administration of a potent anticoagulant on top of budiodarone will further reduce stroke risk, and likely below the age-adjusted overall stroke risk, i.e., lower than the general population not specifically diagnosed with AF. Since budiodarone is primarily metabolized by esterases, synergistic lowering of stroke rate with an effective anticoagulant should not be due to pharmacokinetic interactions raising the effective dose of anticoagulant (with coincident increase in bleeding risk and further drug-drug interactions in this patient group highly susceptible to polypharmacy), but rather pharmacodynamic synergy.

Patient GCI Scale showed significant dose responses, with 60.0% of the patients in the ITT population reporting that they were a lot or completely satisfied with the test medication and 46.7% of patients in the ITT population reporting a lot of improvement or complete relief in the mITT population. There were few significant findings for the AFSS survey. There was a positive trend in the more common AF symptoms of palpitations and shortness of breath during physical activity when analysis was restricted to symptomatic patients only.

All doses of ATI-2042 in this study were well tolerated with an acceptable overall TEAE profile. The most frequent TEAEs were changes in INR values. Increases in INR are expected given that ATI-2042 is an inhibitor of cytochrome P450 2C9 (CYP2C9), which is a primary metabolism pathway for warfarin, and decreased INR values were most likely caused by dose adjustments made to lower increased INRs. Thyroid function changes related to known effects of the drug were mild and reversible, and in only one case led to discontinuation of the study medication. There were no dose-related changes in hematological tests, testicular function tests and the great majority of chemistry tests. There was a reversible elevation in creatinine that is undoubtedly due to inhibition of tubular secretion and did not result in any reduction in glomerular filtration as evidenced by unchanged BUN levels. There was a mild, reversible increase in ALT that appeared to be dose related and was not associated with signs of cholestasis or other signs of liver injury. There were no notable changes in vital signs, physical examinations, ECGs, eye examinations, chest X-rays, and pulmonary function tests, and no safety concerns were identified with ATI-2042 treatment. No evidence of amiodarone-like end-organ toxicity was seen (pulmonary fibrosis, corneal deposits, neuropathy, photosensitivity). There was no evidence of budiodarone tissue accumulation based on lack of corneal microdeposits on slit lamp examination at the end of the treatment period.

The study achieved its primary objectives and demonstrated the efficacy of ATI-2042 at 400 mg BID and 600 mg BID in reducing AT/AF burden in patients with PAF, compared to placebo, for 12 weeks of treatment.

The primary statistical efficacy analysis in the mITT population showed significance for ATI-2042 at the 400 mg BID (p=0.015) and 600 mg BID (p=0.005) doses. The AT/AF burden in these two treatment groups was reduced from baseline by a median of 54% and 75%, respectively. Although the 200 mg BID dose decreased AFB by 10%, that did not reach statistical significance.

The analysis of primary endpoint adjusted for baseline burden confirmed that the drug was efficacious independently of the baseline AF burden.

The overall dose response effect was both robust and linear with p=0.0001. Randomization was balanced across all four treatment groups.

The benefit of ATI-2042 on the larger ITT population in reducing AFB was also highly significant. The percentage reduction in AFB for the 400 mg BID group was 54% (p=0.013) and for the 600 mg BID group it was 74% (p=0.001). A similarly marked dose response effect was seen (p<0.0001).

The efficacy of 600 mg BID in the ITT population was also demonstrated for the number and duration of AT/AF episodes, and the mean duration of NSR. There was a significant reduction in both the number and duration of AT/AF episodes and a corresponding increase in mean duration of NSR. These results are supportive of the primary endpoint.

The efficacy analysis also included a month-by-month assessment of the patients' burden. The reduction in AFB was statistically significant in each of the 3 months of treatment in both the 400 mg BID group and the 600 mg BID group in the ITT population. As the dose and duration of treatment of ATI-2042 was increased, the effect of the drug in reducing AFB became more pronounced. The maximal effect was seen in the third month on 600 mg BID when the median percentage reduction was 83% (p=0.009).

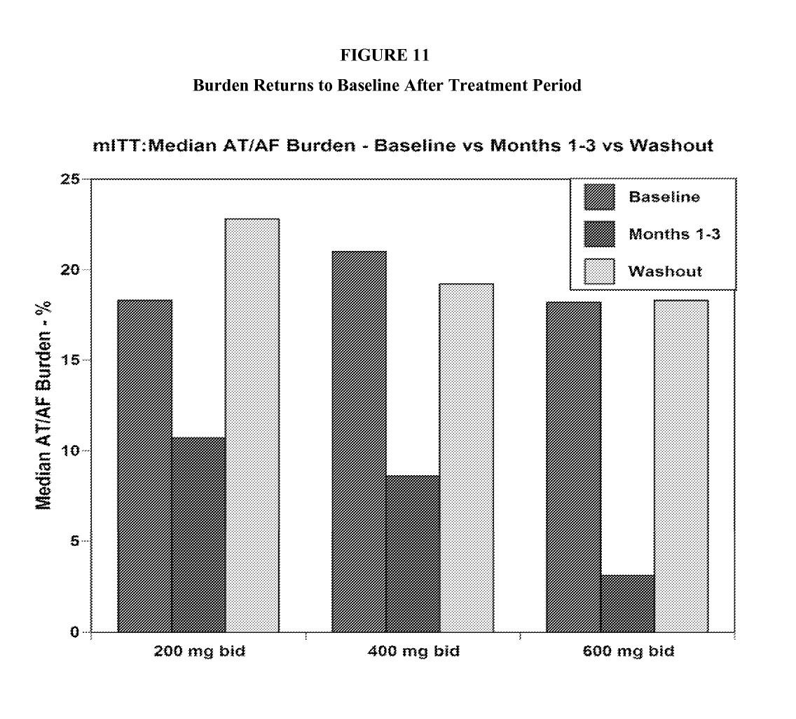

In each of the three ATI-2042 dose groups, the AFB returned to essentially the baseline values within the one-month washout period. The washout data showed no evidence of accumulation or of a rebound effect in the AFB (FIG. 11).

The ITT population achieved statistically significant satisfaction with test medication for all doses tested and reported statistically significant control of atrial fibrillation symptoms in the 400 mg BID and 600 mg BID groups.

The safety of ATI-2042 was demonstrated in this study by the mild TEAE profile. Only one SAE was possibly related to ATI-2042 (hematuria and high INR) and the discontinuation rate was low and balanced across the treatment groups.

The results on thyroid function were not unexpected since ATI-2042 shares the same iodinated chemical structure as amiodarone. Amiodarone inhibits the metabolic conversion of T4 to T3 and it is presumed that ATI-2042 acts in a similar fashion. Lowering levels of T3 and TSH elevations that were observed in the study were consistent with this mechanism and with findings from the previous studies. With the exception of one patient on 200 mg BID who discontinued due to hyperthyroidism (limited to changes in laboratory values only) the effects on thyroid function were considered mild and did not affect study participation.

INR was monitored closely in this study to allow adjustment of the warfarin dose as needed for patients on concomitant therapy. Time within normal range improved in all active drug groups as the initial dose adjustments were made during the first month of treatment. The early increase in INR is expected after start of dosing based on the ability of ATI-2042 to inhibit CYP2C9.

Chest X-ray and pulmonary function tests were performed during the study to monitor for the symptoms of amiodarone pulmonary toxicity. No safety concern was identified.

Because amiodarone is known to accumulate in the cornea and form microcrystalline deposits, slit-lamp examinations were performed. No patient treated with ATI-2042 developed corneal deposits during the study. Together with the washout period data, these findings provide evidence of the lack of tissue accumulation of ATI-2042 and its metabolites.

This, budiodarone significantly reduced AFB at doses of 400 mg BID and 600 mg BID, and appeared to be safe and tolerable at these doses. This was the first controlled study to use permanently implanted pacemakers to continuously record AFB. Use of AFB as a continuously recorded variable offers several advantages. It allows a patient to serve as his or her own control; it accounts for both symptomatic and asymptomatic episodes, thus providing a more accurate measure of clinical response than measurements triggered only by symptoms; and it allows establishment of a dose-response curve with fewer patients than, for example, traditional time to first symptomatic recurrence studies.

The patients recruited for this study had a mean AT/AF burden at screening that ranged from a 17.8% to 23.4%. This is a significant amount of time spent in atrial fibrillation as opposed to normal sinus rhythm. The time spent in atrial fibrillation, especially long duration episodes, has been correlated with increased risk of stroke. For a patient who is experiencing symptoms of atrial fibrillation, this amount of AF also may present a serious quality of life issue.

ATI-2042 was well tolerated, including in CHF NYHA Class I and II patients in this study, and did not exhibit any of the side effects associated with amiodarone accumulation in peripheral tissues. There were no corneal deposits, no evidence of pulmonary toxicity or photosensitivity.

The significant reduction of AFB, shortening of atrial fibrillation episodes and increase in duration of normal sinus rhythm achieved by ATI-2042 in this study thus suggests that budiodarone can offer a considerable advantage for the patient.

### Example C

### PASCAL: A Randomized, Double-Blind, Placebo-Controlled Study of Budiodarone (ATI-2042) in Patients with Paroxysmal Atrial Fibrillation and Pacemakers with Atrial Fibrillation Data Logging Capabilities

Example C is adapted from Ezekowitz et al. PASCAL: a randomized double-blind, placebo-controlled study of budiodarone (ATI-2042) in patients with paroxysmal atrial fibrillation and pacemakers with atrial fibrillation data logging capabilities [abstract]. Presented at Heart Rhythm Society Annual Scientific Sessions, Boston, Mass., May 2009.

Introduction:

PASCAL was conducted to investigate the safety and efficacy of budiodarone (ATI-2042) in patients with paroxysmal atrial fibrillation (PAF). Budiodarone is a chemical analog of amiodarone that shares its mixed ion channel electrophysiological effects but has a different metabolic pathway. The main advantage of budiodarone over amiodarone is its shorter half-life and greatly reduced dependence on CYP450 for clearance. Consequently, budiodarone is not expected to cause side effects associated with tissue accumulation.

The design of PASCAL took advantage of the capability of dual-chamber pacemakers (PM) to detect and log atrial fibrillation data. The primary study endpoint, % change from baseline in atrial fibrillation burden (AFB) at 12 weeks, was based on a continuous rather than discrete variable (time to AF recurrence).

Methods:

Patients with PAF and dual-chamber PM capable of recording and storing electrograms were eligible for the study. After discontinuation of all antiarrhythmic drugs for >5 half-lives, PM programming was adjusted to optimize detection of AF. Following 4 weeks of baseline observation, patients with AFB between 3-70% were randomized to placebo, 200, 400 or 600 mg BID of budiodarone for 12 weeks, followed by 4 weeks of washout. PMs were interrogated every 4 weeks. Full PM downloads including stored electrograms were adjudicated by an independent core lab blinded to treatment assignment. Safety and laboratory assessments were done every 4 weeks.

Results:

Out of 167 consented patients 110 were enrolled. Enrolled patients had mean age of 71, 54% were male, had mean duration of PAF of 3.1 years, 15% had prior cardioversion and 17% had prior ablation, 71% were symptomatic and 56% were taking rate-control drugs. At the end of baseline period, 72 patients met randomization criteria, 61 completed the study. Eleven patients terminated the study early due to protocol violations (4) or adverse events (7). The primary efficacy endpoint achieved statistical significance in the 400 mg (p=0.015) and 600 mg (p=0.006) dose groups. The median reduction in AFB in these groups was 54.4% and 75.5%, compared to placebo respectively. The dose-response was statistically significant (p=0.0001). There was no statistically significant difference in AFB between baseline and washout period. Secondary endpoints of number and duration of AT/AF episodes were reduced; mean duration of normal sinus rhythm increased. Safety was acceptable with no drug-related SAEs.

### CONCLUSIONS

Dual-chamber PMs were successfully used for monitoring AFB in a blinded drug trial. Budiodarone significantly reduced AFB by up to 75.5% over 12 weeks of treatment while exhibiting a benign safety profile.

1. A solution comprising benzyl alcohol and budiodarone, wherein the budiodarone has an aqueous solubility of about 200 mg/ml to about 1250 mg/ml when contacted with an aqueous solution.

2. The solution of claim 1, wherein the aqueous solubility of budiodarone in the solution is 200 mg/ml to 750 mg/ml.

3. The solution of claim 1, wherein the aqueous solubility of budiodarone in the solution is 750 mg/ml to 1250 mg/ml.

4. The solution of claim 1, wherein the benzyl alcohol and budiodarone are in a molar ratio of benzyl alcohol to budiodarone of between about 0.5 to 1 and 4.5 to 1.

5. The solution of claim 4, wherein the solution has characteristics of a solution prepared by:
(a) mixing benzyl alcohol, budiodarone, and a co-solvent in a sufficient amount to make a clear solution, wherein the benzyl alcohol and budiodarone are present in a molar ratio that is greater than between about 0.5 to 1 and 4.5 to 1; and
(b) removing the co-solvent until the solution comprises benzyl alcohol and budiodarone in a molar ratio of benzyl alcohol to budiodarone of between about 0.5 to 1 and 4.5 to 1.

6. The solution of claim 4, wherein the solution has characteristics of a solution prepared by:
(a) mixing benzyl alcohol, budiodarone, and water, wherein the benzyl alcohol and budiodarone are present in a molar ratio that is greater than between about 0.5 to 1 and 4.5 to 1; and
(b) removing the water by lyophilization until the formulation comprising benzyl alcohol and budiodarone in a molar ratio of benzyl alcohol to budiodarone of between about 0.5 to 1 and 4.5 to 1 is formed.

7. (canceled)

8. The solution of claim 1, wherein the benzyl alcohol and budiodarone are present in a molar ratio of benzyl alcohol to budiodarone of between about 2 to 1 and 4 to 1.

9. The solution of claim 1, wherein the benzyl alcohol and budiodarone are present in a molar ratio of benzyl alcohol to budiodarone of about 3 to 1.

10. The solution of claim 1, wherein the benzyl alcohol and budiodarone are present in a molar ratio of benzyl alcohol to budiodarone of between about 5 to 1 and 11 to 1.

11-17. (canceled)

18. An aqueous solution comprising benzyl alcohol and budiodarone, wherein the budiodarone is present in the solution in a concentration of about 200 mg/ml to about 1250 mg/ml.

19. The solution of claim 18, wherein the concentration of budiodarone in the solution is between about 500 and 1250 mg/ml.

20. The solution of claim 18, wherein the concentration of budiodarone in the solution is between about 800 and 1250 mg/ml.

21. An aqueous solution comprising benzyl alcohol and budiodarone, wherein the budiodarone is present in the solution at a concentration of between about 0.1 and 500 mg/ml.

22-27. (canceled)

28. A capsule comprising the solution of claim 1.

29-31. (canceled)

32. An oral syrup comprising the solution of claim 1 and pharmaceutically acceptable excipients.

33. A kit comprising the solution of claim 1 and instructions for use.

34. (canceled)

35. A process for preparing a solution comprising benzyl alcohol and budiodarone in a molar ratio of benzyl alcohol to budiodarone of between about 0.5 to 1 and 4.5 to 1, wherein the budiodarone has an aqueous solubility of about 200 mg/ml to about 1250 mg/ml when contacted with an aqueous environment, and wherein the process comprises:
(a) mixing benzyl alcohol, budiodarone, and a co-solvent in a sufficient amount to make a clear solution, wherein the benzyl alcohol and budiodarone are present in a molar ratio that is greater than between about 0.5 to 1 and 4.5 to 1; and
(b) removing the co-solvent until the solution comprises benzyl alcohol and budiodarone in a molar ratio of benzyl alcohol to budiodarone of between about 0.5 to 1 and 4.5 to 1.

36-40. (canceled)

41. A process for preparing a solution comprising benzyl alcohol and budiodarone in a molar ratio of benzyl alcohol to budiodarone of between about 0.5 to 1 and 4.5 to 1, wherein the budiodarone has an aqueous solubility of about 200 mg/ml to about 1250 mg/ml when contacted with an aqueous environment, and wherein the process comprises:
(a) mixing benzyl alcohol, budiodarone, and water, wherein the benzyl alcohol and budiodarone are present in a molar ratio that is greater than between about 0.5 to 1 and 4.5 to 1; and
(b) removing the water by lyophilization until the formulation comprising benzyl alcohol and budiodarone in a molar ratio of benzyl alcohol to budiodarone of between about 0.5 to 1 and 4.5 to 1 is formed.

42. (canceled)

43. A method of treating cardiac arrhythmia or reducing the incidence of cardiac arrhythmias comprising administering to a patient in need thereof the solution of claim 1.

44. A method of restoring sinus and ventricular rhythms in a patient having persistent atrial fibrillation, paroxysmal atrial fibrillation, atrial flutter, ventricular arrhythmias, or ventricular fibrillation comprising administering to a patient in need thereof the solution of claim 1.

45-56. (canceled)

57. A composition comprising benzyl alcohol and budiodarone, wherein the benzyl alcohol and budiodarone are present in a molar ratio of benzyl alcohol to budiodarone of about 5 to 1.
